(19) United States
(12) Patent Application Publication
(10) Pub. No.: US 2024/0207042 A1
(43) Pub. Date: Jun. 27, 2024

(54) RETINAL IMPLANT INSERTION TOOL

(71) Applicant: PRECISE BIO 3D LTD, Modiin Maccabim Reut (IL)

(72) Inventors: Yossi BOUKOBZA, Nataf (IL); Amos EITAN, Jerusalem (IL); Ariel EISENBACH, Zifum (IL); Eliahu ELIACHAR, Haifa (IL); Michal MARCUS, Kochav Yair (IL); Gilad RABINA, Tel Aviv (IL); Lior ROSENBERG BELMAKER, Shoham (IL); Amit SEGAL, Afula (IL); Dorin SADE YAZDI, Tel Aviv (IL)

(21) Appl. No.: 18/682,924

(22) PCT Filed: Aug. 7, 2022
(86) PCT No.: PCT/IL2022/050858

§ 371 Date: Feb. 11, 2024

Related U.S. Application Data
(60) Provisional application No. 63/232,474, filed on Aug. 12, 2021.

(51) Int. Cl. A61F 2/14; A61F 9/00; A61L 27/36

(52) U.S. Cl. CPC A61F 2/148; A61F 2/142; A61F 9/0017; A61L 27/3604; A61L 27/3675; A61L 2430/16

(57) ABSTRACT

The present invention provides an insertion tool for inserting an implant, said implant having an implant lower side and an implant upper side, said implant upper side comprising cells, said insertion tool comprising: an implant carrier configured to hold said implant; and a body having at least two configurations, a first body configuration with said implant carrier at least partially contained within said body and said implant held by said implant carrier and a second body configuration with said implant carrier outside said body; said implant carrier is configured to maintain a position of the implant and a shape of the implant and functionality of said cells during such time as the implant carrier is contained by said body, and during an implantation procedure.

### FIELD OF THE INVENTION

The present invention generally pertains to a system and method for insertion of a retinal implant under the retina.

### BACKGROUND OF THE INVENTION

Millions of people around the world suffer from retinal degenerative diseases with varying degrees of vision loss including complete blindness. One of the most common retinal degenerative diseases is age-related macular degeneration (AMD), which mostly affects individuals over the age of 55. Currently, over 200 million people worldwide are affected by AMD, with approximately 10% at an advanced stage of the disease. Since aging is one of the main risk factors for AMD, these numbers are likely to increase dramatically as life expectancy continues to rise. AMD has two advanced stages: dry AMD, or geographic atrophy, and wet AMD, or choroidal neovascularization. Wet AMD is treated with antiangiogenic monthly injections inhibiting blood vessel growth into the retina, whereas, for dry AMD, there is to date no treatment. Histologically, the dry form of AMD is characterized by the death of retinal pigment epithelium (RPE) cells in the macular region of the eye, which lead to death of the photoreceptors that provide the high-acuity vision needed for face recognition, driving, etc. The disease etiology starts with death of the RPE cells that provide photoreceptors with functional support. As the disease progresses, there is irreversible photoreceptor cell loss affecting central vision (macular region), making patients handicapped. Therefore, possible treatments for dry AMD could include transplanting an RPE monolayer prior to photoreceptor loss or transplanting an implant that includes both a layer of RPE cells and photoreceptor cells.

There are several RPE cell therapies in various stages of development, either as a suspension or as a monolayer implant, aimed at treating dry AMD (Sharma R, Bose D, Maminishkis A, Bharti K. Retinal Pigment Epithelium Replacement Therapy for Age-Related Macular Degeneration: Are We There Yet? Annu Rev Pharmacol Toxicol. 2020 Jan. 6; 60:553-572. doi: 10.1146/annurev-pharmtox-010919-023245. PMID: 31914900.).

Retinal implants are devices for restoring at least partial sight to persons whose retinal pigment epithelium (RPE) cells, retinal photoreceptors or both are degenerating and there is retinal atrophy. Typically, a retinal implant is less than 10 mm in size and is less than about 100 μm thick.

Some examples of prior art retinal implants and insertion tools are given below.

Ben M'Barek, working in France and Pittsburgh, USA, have developed (2020) an implant using decellularized human amniotic membrane for the scaffold. The implant is 3×5 mm, inserted by means of an 18 Ga tool. The insertion tool enters the retina via a 2 mm retinotomy incision.

Hinton & Clegg (See Kashani et al), working at Regenerative Patch Technologies in Menlo Park, CA, USA, have developed (2019) a Phase I-II RPE patch using Parylene for the scaffold. The implant is 3.5×6.25 mm. The tool has an OD of 1.47 mm and is inserted by means of an 18 Ga tool. The insertion tool enters the retina via a 1 mm retinotomy incision.

WIPO patent publication number WO2012/004592 to da Cruz & Coffey, working in the UK, discloses an implant using a Vitronectin-coated polyethylene terephthalate (PET) membrane for the scaffold. The latest work by da Cruz and Coffey is from 2019. The implant is 3×6 mm, inserted by means of a tool between 0.5-4 mm.

Mandai, working in Japan and San Francisco, USA, have developed an implant using type I collagen gel as temporary scaffold. The implant is 1.3×3 mm, inserted by means of a 20 Ga tool.

Sharma & Bharti, working at the NIH in the USA, have developed (2020) an implant using Poly-lactic-coglycolic acid (PLGA) for the scaffold. The implant is 2×4 mm, inserted via a 2.5 mm retinotomy incision.

For this reason, a large delivery device which requires a large retinotomy is disadvantageous, as is a delivery device that does not allow safe manipulation and delivery of a thin retinal implant comprised of RPE cells or RPE and photoreceptor cells to the subretinal space. A further disadvantage of prior-art delivery devices is that they do not properly protect the cell layers on the implant during delivery.

It is therefore a long felt need to provide a tool for subretinally emplacing a retinal implant that does only minimal damage to the eye or the retina, that does not damage the implant, and where the position of the implant and the angle of the implant relative to the retina is not uncontrollable.

The corneal endothelium is the posterior-most layer of the cornea. The endothelial cells are vital to maintaining corneal transparency for a clear vision. Corneal endothelial cells create a monolayer of polygonal cells connected by tight junctions, which continuously pump fluid out of the cornea, keeping it in a properly hydrated and in a transparent state. Dysfunction of the endothelium leads to edema and blindness. This can be reversed using corneal transplantation. To date, several approaches have been employed for the treatment of endothelium dysfunction, the two most common approaches are Descemet's stripping (automated) endothelial keratoplasty (DSEK/DSAEK) and Descemet's Membrane Endothelial Keratoplasty (DMEK). In both surgical approaches, the patient's endothelial layer and the Descemet's membrane are removed, with the difference being the thickness of the implanted tissue: DSEK/DSAEK includes some of the stromal layer, the Descemet membrane and endothelium, and, for DMEK, only the Descemet membrane and endothelium (thickness around 20 μm) are removed. DMEK is gaining popularity as thinner grafts have been associated with quicker visual rehabilitation and better overall visual outcomes (see references: Busin M, Madi S, Santorum P, Scorcia V, Beltz J., Ultrathin descemet's stripping automated endothelial keratoplasty with the microkeratome double-pass technique: two-year outcomes. Ophthalmology. 2013 June; 120(6):1186-94 and Romano V, Steger B, Myneni J, Batterbury, M, Willoughby, CE, Kaye, SB, Preparation of ultrathin grafts for Descemet-stripping endothelial keratoplasty with a single microkeratome pass. J Cataract Refract Surg 2017 January; 43(1):12-15. doi: 10.1016/j.jcrs.2016.12.009).

Nevertheless, due to a shortage of donors, there is a worldwide shortage of high-quality corneal tissue, currently obtained exclusively from organ donors. To meet this need, a tissue-engineered corneal graft for endothelium dysfunction can be used, mimicking the natural characteristics of the human endothelium. Such an engineered implant is usually composed of a thin scaffold film, covered by a monolayer of human corneal endothelial cells, derived or originated from cadavers or human stem cells, respectively. The typical diameter of a corneal graft or an engineered implant is between 6 mm and 9 mm, with thicknesses ranging from 5 μm to 150 μm.

The surgical procedure includes removal of the native dysfunctional corneal endothelium and the adjacent Descemet's membrane from the patient's corneal stroma, followed by transplantation of the corneal graft or engineered implant. To perform this procedure, a cut is made in the periphery of the cornea or in the sclera, through which an implantation device is inserted into the anterior chamber of the eye.

It has been shown that a thin graft or implant (below 50 μm) and a minimal incision for implant delivery (diameter smaller than 4 mm) provide quicker and better medical outcomes after surgery. It is also known that, to keep the graft or the implant highly functional, it is mandatory not to touch or scrape the endothelial cells with any tool during the procedure. In addition, the engineered implant is thin and is difficult to handle, similar to cadaver-derived DMEK implants. However, the commercially available tools are suboptimal, either providing the physician with low implantation control, requiring a highly-skilled operator, or requiring destructive handling.

It is therefore a long felt need to provide a tool for implanting a thin corneal graft or a corneal implant.

### SUMMARY OF THE INVENTION

It is an object of the present invention to disclose a tool for insertion of a retinal implant under the retina.

It is another object of the present invention to disclose an insertion tool for inserting an implant, said implant having an implant lower side and an implant upper side, said implant upper side comprising cells, said insertion tool comprising:
an implant carrier configured to hold said implant; and
a body having at least two configurations, a first body configuration with said implant carrier at least partially contained within said body and said implant held by said implant carrier and a second body configuration with said implant carrier outside said body;
said implant carrier being in mechanical communication with said body via a carrier arm selected from a group consisting of a wire, a ribbon, a closed tube, an open tube and any combination thereof;
wherein said implant carrier is configured to maintain a position of the implant and a shape of the implant and functionality of said cells during such time as the implant carrier is contained by said body, and during an implantation procedure;
wherein said implant carrier has at least two configurations, a first carrier configuration where the implant carrier has a shape selected from a group consisting of folded, rolled and bent, and a second carrier configuration where the implant carrier has a shape selected from a group consisting of unfolded, curved, flat and straight;
further wherein said implant carrier is in said first carrier configuration at such times as said implant carrier is within said body and said implant carrier is transformable to said second carrier configuration during such times as said implant carrier is outside of said body.

It is another object of the present invention to disclose the tool as described above, wherein said implant carrier is retractable into said body.

It is another object of the present invention to disclose the tool as described above, wherein said implant carrier is movable outward from said body.

It is another object of the present invention to disclose the tool as described above, wherein said body is openable.

It is another object of the present invention to disclose the tool as described above, wherein said implant carrier is a part of said body.

It is another object of the present invention to disclose the tool as described above, wherein said implant carrier comprises a member of a group consisting of Nitinol, Polyimide (Kapton), stainless steel and any combination thereof.

It is another object of the present invention to disclose the tool as described above, wherein said implant carrier comprises a coating selected from a group consisting of a hydrophilic coating, a hydrophobic coating and any combination thereof.

It is another object of the present invention to disclose the tool as described above, wherein said implant is made of a material selected from a group consisting of soft, foldable, bendable and any combination thereof.

It is another object of the present invention to disclose the tool as described above, additionally comprising an implant removal mechanism selected from a group consisting of an arm contactable to said implant, a wire attachable to said implant, fluid flow, vibration, and any combination thereof.

It is another object of the present invention to provide the tool as defined above, wherein said fluid flow is adapted to perform at least one selected from a group consisting of lifting said implant from said implant carrier, pushing said implant out of said tool, remove all air bubbles in said tool, and any combination thereof.

It is another object of the present invention to provide the tool as defined above, additionally comprising at least one element, adapted to direct said fluid flow to a predetermined direction.

It is another object of the present invention to provide the tool as defined above, additionally comprising at least one element, adapted to elevate said implant from said graft carrier, allowing said fluid to flow below said implant.

It is another object of the present invention to provide the tool as defined above, wherein said vibration is provided by at least one selected from a group consisting of tapping, electric motor, piezoelectric transducer, ultrasound means and any combination thereof.

It is another object of the present invention to provide the tool as defined above, additionally comprising means adapted to increase said fluid flow velocity.

It is another object of the present invention to provide the tool as defined above, wherein said increase in said fluid flow velocity is provided by means selected from a group consisting of narrowing said body, enlarging said body, blocking part thereof, adding an elevated or protruding part inside or at the opening of said body, or any combination thereof.

It is another object of the present invention to provide the tool as defined above, wherein said fluid flow is controlled either manually or automatically.

It is another object of the present invention to disclose the tool as described above, wherein said implant carrier is configured to position said implant into a member of a group consisting of a subretinal bleb and an anterior chamber.

It is another object of the present invention to disclose the tool as described above, wherein said implant is configured for ophthalmic applications.

It is another object of the present invention to disclose the tool as described above, wherein said implant is a retinal implant.

It is another object of the present invention to disclose the tool as described above, wherein said implant is a corneal implant.

It is another object of the present invention to disclose the tool as described above, wherein said implant comprises a member of a group consisting of retinal pigment epithelium cells, photo-receptor cells and any combination thereof.

It is another object of the present invention to disclose the tool as described above, wherein said implant comprises a member of a group consisting of collagen, hydrogel, a medication and any combination thereof.

It is another object of the present invention to disclose the tool as described above, wherein said medication is a slow-release medication.

It is another object of the present invention to disclose the tool as described above, wherein said implant diameter is in a range of 0.5 mm to 8 mm.

It is another object of the present invention to disclose the tool as described above, wherein said implant thickness is in a range of 2 μm to 500 μm.

It is another object of the present invention to disclose the tool as described above, wherein said implant length is in a range of 0.5 mm to 8 mm.

It is another object of the present invention to disclose the tool as described above, wherein said implant width is in a range of 0.5 mm to 8 mm.

It is another object of the present invention to disclose the tool as described above, additionally comprising a cannula.

It is another object of the present invention to disclose the tool as described above, wherein said cannula has a diameter in a range of 0.7 mm to 4 mm.

It is another object of the present invention to disclose the tool as described above, wherein said cannula is insertable through a trocar.

It is another object of the present invention to disclose the tool as described above, wherein the trocar diameter is no larger than 20 G (0.9 mm).

It is another object of the present invention to disclose the tool as described above, wherein the trocar diameter is larger than 20 G (0.9 mm).

It is another object of the present invention to disclose the tool as described above, wherein actuation of removal of said implant from said implant carrier is effectuated by a mechanism comprising a member of a group consisting of a slider, a pin, a converter from circular motion to linear motion, a motor and gear, a motor and cam, a wheel, a knob, a screw, and any combination thereof.

It is another object of the present invention to disclose the tool as described above, additionally comprising a protective seal at a distal end of said insertion tool.

It is another object of the present invention to disclose the tool as described above, wherein said protective seal is selected from a group consisting of a cap, a diaphragm and any combination thereof.

It is another object of the present invention to disclose the tool as described above, wherein said protective seal allows at least one of a group consisting of diffusion of gas therethrough and a nutrient path therethrough.

It is another object of the present invention to disclose the tool as described above, wherein said insertion tool is configured to enable fluid flow to perform a member of a group consisting of detaching said implant from said implant carrier, positioning said implant, transferring liquid, maintaining viability of said implant, maintaining intraocular pressure, maintaining retinal bleb size, and any combination thereof.

It is another object of the present invention to disclose the tool as described above, wherein said implant is emplaceable within said insertion tool prior to shipment and said implant is maintainable in a usable condition between said emplacing and use.

It is another object of the present invention to disclose the tool as described above, wherein said implant is maintainable in a temperature range of 4° C. to 25° C. between said emplacing and said use.

It is another object of the present invention to disclose the tool as described above, wherein said implant is maintainable in a temperature range of 30° C. to 38° C. between said emplacing within said insertion tool and said use.

It is another object of the present invention to disclose the tool as described above, wherein said implant is maintainable in solution between said emplacing within said insertion tool and said use.

It is another object of the present invention to disclose the tool as described above, wherein said fluid comprises a member of a group consisting of balanced saline solution (BSS), BSS Plus, BSS Plus with additional viscous content such as Hyaluronic acid, and a CO₂ independent medium.

It is another object of the present invention to disclose the tool as described above, wherein a shape of a portion of said body is selected from a group consisting of bent, controllably bendable, straight and any combination thereof.

It is another object of the present invention to disclose the tool as described above, wherein said bent or controllably bendable portion is passable through at least one of a trocar and a cannula.

It is another object of the present invention to disclose the tool as described above, wherein, in said bent configuration, an angle between a longitudinal axis of said body and a longitudinal axis of said bent portion is in a range from 0° to 90°.

It is another object of the present invention to disclose the tool as described above, wherein, in said bent configuration, an angle between a longitudinal axis of said body and a longitudinal axis of said bent portion is in a range from 20° to 90°.

It is another object of the present invention to disclose the tool as described above, wherein, in said bent configuration, an angle between a longitudinal axis of said body and a longitudinal axis of said bent portion is in a range from 10° to 50°.

It is another object of the present invention to disclose the tool as described above, wherein, in said bent configuration, an angle between a longitudinal axis of said body and a longitudinal axis of said bent portion is 30°.

It is another object of the present invention to disclose the tool as described above, wherein at least a portion of a member of a group consisting of said body, said cap, said implant carrier and any combination thereof is transparent.

It is another object of the present invention to disclose the tool as described above, wherein said transparency enables visibility of at least a portion of a member of a group consisting of said implant, said implant carrier and any combination thereof.

It is another object of the present invention to disclose the tool as described above, wherein at least a portion of said insertion tool is colored to improve visibility of a member of a group consisting of said implant, at least a portion of a component of said insertion tool and any combination thereof.

It is another object of the present invention to disclose the tool as described above, wherein said color is selected from a group consisting of darker than another component, lighter than another component, fluorescent, green, and any combination thereof.

It is another object of the present invention to disclose the tool as described above, wherein control of said insertion tool is selected from a group consisting of robotic, automatic, semi-automatic, manual, vibration damping and any combination thereof.

It is another object of the present invention to disclose the tool as described above, additionally comprising a lighting mechanism configured to provide light at a distal end of said insertion tool.

It is another object of the present invention to disclose the tool as described above, wherein said lighting mechanism comprises a member of a group consisting of an LED, a fiber optic cable, and any combination thereof.

It is another object of the present invention to disclose the tool as described above, additionally comprising a camera configured to display a view of a region near a distal portion of said insertion tool.

It is another object of the present invention to disclose the tool as described above, additionally comprising a mechanism for rotating said implant while maintaining a direction of implantation.

It is another object of the present invention to disclose the tool as described above, wherein said implant is ejectable from said implant carrier.

It is another object of the present invention to disclose the tool as described above, wherein said implant carrier is in contact with only said implant lower side.

It is another object of the present invention to disclose the tool as described above, wherein said implant carrier is configured to protect said implant upper side from mechanical stress and shear.

It is another object of the present invention to disclose the tool as described above, wherein the implant is releasably connectable to the implant carrier, said releasable connection configured, before release, to prevent a member of a group consisting of movement of said implant, folding of said implant and any combination thereof.

It is another object of the present invention to disclose the tool as described above, additionally comprising a locking mechanism.

It is another object of the present invention to disclose the tool as described above, wherein said locking mechanism comprises a member of a group consisting of locking tubes comprising holes through which a fluid can be passed, an arm contactable to said implant, a wire attachable to said implant, and any combination thereof,

It is another object of the present invention to disclose the tool as described above, wherein said insertion tool is configured to allow flow of flow fluid into said insertion tool from a volume near a distal end of said insertion tool.

It is another object of the present invention to disclose the tool as described above, wherein at least a portion of said insertion tool is configured to enable implant orientation by means of rotation of said at least a portion of said insertion tool.

It is another object of the present invention to disclose the tool as described above, additionally comprising an insertion stopper, configured to allow adjustment of a distance said implant carrier extends distally from said body of said insertion tool.

It is another object of the present invention to disclose the tool as described above, wherein said implant carrier is extendable from said body by at least one predetermined distance.

It is another object of the present invention to disclose the tool as described above, wherein said at least one predetermined distance comprises a first predetermined distance and a second predetermined distance.

It is another object of the present invention to disclose the tool as described above, wherein said first predetermined distance is 8.5 mm.

It is another object of the present invention to disclose the tool as described above, wherein said second predetermined distance is 4 mm.

It is another object of the present invention to disclose the tool as described above, wherein said first predetermined distance is in a range from 7 mm to 10 mm and said second predetermined distance is in a range from 2 to 6 mm.

It is another object of the present invention to disclose the tool as described above, wherein said first predetermined distance is in a range from 7 mm to 10 mm and said second predetermined distance is in a range from 2 to 5.5 mm.

It is another object of the present invention to disclose the tool as described above, wherein said extending mechanism is selected from a group consisting of a slider, a pin, a converter from circular motion to linear motion, a motor and gear, a motor and cam, and any combination thereof.

It is another object of the present invention to disclose the tool as described above, wherein said carrier tube comprises a superelastic material.

It is another object of the present invention to disclose the tool as described above, wherein said carrier tube comprises Nitinol.

It is another object of the present invention to disclose the tool as described above, wherein said implant carrier comprises a superelastic material.

It is another object of the present invention to disclose the tool as described above, wherein said implant carrier comprises Nitinol.

It is another object of the present invention to disclose the tool as described above, wherein said implant carrier comprises holes.

It is another object of the present invention to disclose the tool as described above, wherein said implant carrier comprises a member of a group consisting of an anchoring structure, a compliance mechanism and any combination thereof.

It is another object of the present invention to disclose the tool as described above, wherein said anchoring structure contacts said implant at a location on said implant selected from a group consisting of a proximal portion of said implant, a central portion of said implant, a distal portion of said implant and any combination thereof.

It is another object of the present invention to disclose the tool as described above, wherein said anchoring structure is configured to remove said implant from said implant carrier by a means selected from a group consisting of pulling said implant from said implant carrier and pushing implant from said implant carrier.

It is another object of the present invention to disclose the tool as described above, wherein said anchoring structure is configured to enable release of said implant from said implant carrier by ending of contact between said holding member and said implant.

It is another object of the present invention to disclose the tool as described above, wherein said anchoring structure comprises at least one tooth.

It is another object of the present invention to disclose the tool as described above, wherein said anchoring structure retains said implant on said implant carrier before use.

It is another object of the present invention to disclose the tool as described above, wherein said anchoring structure lifts said implant off of said implant carrier to allow removal of said implant from said implant carrier.

It is another object of the present invention to disclose the tool as described above, additionally comprising a fluid inlet, said fluid inlet in fluid communication with said implant carrier.

It is another object of the present invention to disclose the tool as described above, additionally comprising a cover tube configured to retain said carrier tube and said arm in said first configuration.

It is another object of the present invention to disclose the tool as described above, wherein said cover tube is movable from said bendable region, removal of said cover tube from said bendable region enabling transfer of said carrier tube and said arm to said second configuration.

It is another object of the present invention to disclose the tool as described above, wherein, in said closed configuration, said implant carrier has a diameter less than that of said carrier tube.

It is another object of the present invention to disclose the tool as described above, wherein, in said open configuration, said implant carrier has a curvature such that a distance from a bottom center of said implant carrier to a top edge of said implant carrier is a range from 0 to 1 cm.

It is another object of the present invention to disclose the tool as described above, wherein, in said open configuration, said implant carrier has a curvature such that a distance from a bottom center of said implant carrier to a top edge of said implant carrier is a range from 0.2 mm to 0.5 mm.

It is another object of the present invention to disclose the tool as described above, wherein said transfer of said implant carrier from said closed position to said open position is reversible.

It is another object of the present invention to disclose the tool as described above, wherein said transfer of said implant carrier from said closed position to said open position is effectuated by a means selected from a group consisting of pulling at least a portion of said implant carrier and pushing at least a portion of said implant carrier.

It is another object of the present invention to disclose the tool as described above, wherein said transfer of said implant carrier from said closed configuration to said open configuration is by means of wires.

It is another object of the present invention to disclose the tool as described above, wherein said wires comprise a shape memory material.

It is another object of the present invention to disclose the tool as described above, wherein said wires comprise Nitinol.

It is another object of the present invention to disclose the tool as described above, wherein, in said open configuration, a length of said implant carrier is in a range from 1.5 mm to 12 mm.

It is another object of the present invention to disclose the tool as described above, wherein, in said open configuration, a width of said implant carrier is in a range from 1.5 mm to 1 cm.

It is another object of the present invention to disclose the tool as described above, wherein said implant carrier is configured to hold a plurality of implants.

It is another object of the present invention to disclose the tool as described above, wherein said implant carrier for a plurality of implants is a case with shelves.

It is another object of the present invention to disclose the tool as described above, wherein said plurality of implants is in a range from 2 to 10.

It is another object of the present invention to disclose the tool as described above, wherein said implant is movable by means of wires.

It is another object of the present invention to disclose the tool as described above, wherein said wires are attachable to said implant by means of a group consisting of ears and holes in said implant.

It is another object of the present invention to disclose the tool as described above, wherein said insertion tool is configured to perform at least one function selected from a group consisting of emplace an ophthalmic implant, emplace an ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof.

It is another object of the present invention to disclose the tool as described above, wherein said carrier arm wire has a width in a range from 50 μm to 0.5 mm.

It is another object of the present invention to disclose the tool as described above, wherein said carrier arm wire has a length in a range from 5 cm to 6 cm.

It is another object of the present invention to disclose the tool as described above, wherein the implant is either loaded into the insertion tool at a manufacturing site or loaded into the insertion tool at the operation site.

It is another object of the present invention to disclose the tool as described above, wherein said insertion tool is configured to provide a slow and constant flow of fluid through the insertion tool.

It is another object of the present invention to disclose the tool as described above, wherein said flow of fluid is in a range of 0.05 ml/min to 1 ml/min.

It is another object of the present invention to disclose the tool as described above, wherein flow of fluid is via said cannula.

It is another object of the present invention to disclose the tool as described above, wherein said implant is loadable into said insertion tool by transforming said insertion tool to said first body configuration.

It is another object of the present invention to disclose the tool as described above, wherein said implant is loadable into said insertion tool by transforming said implant carrier to said first implant carrier configuration.

It is another object of the present invention to disclose the tool as described above, wherein said insertion tool is connectable to an operating room facility, said operating room facility selected from a group consisting of a fluid source, a positioning tool, a power supply, a light source, and any combination thereof.

It is another object of the present invention to disclose the tool as described above, wherein said implant is separated from a larger implant sheet by a means selected from a group consisting of punching, cutting, microdissection, laser ablation and any combination thereof.

It is another object of the present invention to disclose the tool as described above, wherein said separation of said implant from said larger implant sheet is performed while the implant is resting on or located above said implant carrier.

It is another object of the present invention to disclose the tool as described above, wherein a material of said implant carrier is configured to resist damage during laser ablation or microdissection processing to separate said implant from said larger implant sheet by having a property selected from a group consisting of reflecting laser light, being transparent to laser light, being resistive to high temperatures, having a protection layer and any combination thereof.

It is another object of the present invention to disclose the tool as described above, wherein said material of said implant carrier is configured to resist damage during cutting or punching of said implant from said larger implant sheet by having a property selected from a group consisting of having a mechanical yield strain of at least 50%, having an elastic modulus of at least 5 GPa, having a Shore durometer hardness type D of at least 50 and any combination thereof.

It is another object of the present invention to disclose a method of inserting an implant, comprising steps of:
providing an insertion tool for inserting an implant, said implant having an implant lower side and an implant upper side, said implant upper side comprising cells, said insertion tool comprising:
   an implant carrier configured to hold said implant; and
   a body having at least two configurations, a first body configuration with said implant carrier at least partially contained within said body and said implant held by said implant carrier and a second body configuration with said implant carrier outside said body;
      said implant carrier in mechanical communication with said body via a carrier arm selected from a group consisting of a wire, a ribbon, a closed tube, an open tube and any combination thereof;
emplacing said implant on said implant carrier;
making an incision in the eyeball;
inserting an incision tool, said incision tool making an incision in a retina;
removing said incision tool;
inserting a second tool and creating a bleb in said retina, said bleb within said retina and in contact with said incision in said retina;
removing said second tool;
inserting said insertion tool, a tip of said insertion tool passing through said incision;
transferring said body from said first body configuration to said second body configuration;
transferring said implant carrier from a first carrier configuration to a second carrier configuration;
removing said implant from said implant carrier into said bleb;
removing said insertion tool;
removing, by flattening, said bleb;
sealing said incision in said retina; and,
sealing said incision in said eyeball;
   wherein said implant carrier is configured to maintain a position of the implant and a shape of the implant and functionality of said cells during such time as the implant carrier is contained by said body, and during an implantation procedure;
   wherein, in said first carrier configuration, the implant carrier has a shape selected from a group consisting of folded, rolled and bent, and, in said second carrier configuration, the implant carrier has a shape selected from a group consisting of unfolded, curved, flat and straight;
   further wherein said implant carrier is in said first carrier configuration at such times as said implant carrier is within said body and said implant carrier is transformable to said second carrier configuration during such times as said implant carrier is outside of said body.

It is another object of the present invention to disclose the method as described above, additionally comprising a step of retracting said implant carrier into said body.

It is another object of the present invention to disclose the method as described above, additionally comprising a step of moving said implant carrier outward from said body.

It is another object of the present invention to disclose the method as described above, additionally comprising a step of opening said body.

It is another object of the present invention to disclose the method as described above, additionally comprising a step of providing said implant carrier as a part of said body.

It is another object of the present invention to disclose the method as described above, additionally comprising a step of providing said implant carrier comprising a member of a group consisting of Nitinol, Polyimide (Kapton), stainless steel and any combination thereof.

It is another object of the present invention to disclose the method as described above, additionally comprising a step of providing said implant carrier comprising a coating selected from a group consisting of a hydrophilic coating, a hydrophobic coating and any combination thereof

It is another object of the present invention to disclose the method as described above, additionally comprising a step of making said implant of a material selected from a group consisting of soft, foldable, bendable and any combination thereof.

It is another object of the present invention to disclose the method as described above, additionally comprising steps of providing an implant removal mechanism and of selecting said implant removal mechanism from a group consisting of an arm contactable to said implant, a wire attachable to said implant, fluid flow, vibration, and any combination thereof.

It is another object of the present invention to provide the method as defined above, wherein said fluid flow is adapted to perform at least one selected from a group consisting of lifting said implant from said implant carrier, pushing said implant out of said tool, remove all air bubbles in said tool, and any combination thereof.

It is another object of the present invention to provide the method as defined above, additionally comprising at least one element, adapted to direct said fluid flow to a predetermined direction.

It is another object of the present invention to provide the method as defined above, additionally comprising at least one element, adapted to elevate said implant from said graft carrier, allowing said fluid to flow below said implant.

It is another object of the present invention to provide the method as defined above, wherein said vibration is provided by at least one selected from a group consisting of tapping, electric motor, piezoelectric transducer, ultrasound means and any combination thereof.

It is another object of the present invention to provide the method as defined above, additionally comprising means adapted to increase said fluid flow velocity.

It is another object of the present invention to provide the method as defined above, wherein said increase in said fluid flow velocity is provided by means selected from a group consisting of narrowing said body, enlarging said body, blocking part thereof, adding an elevated or protruding part inside or at the opening of said body, or any combination thereof.

It is another object of the present invention to provide the method as defined above, wherein said fluid flow is controlled either manually or automatically.

It is another object of the present invention to disclose the method as described above, additionally comprising a step of configuring said implant carrier to position said implant into a member of a group consisting of subretinal bleb and an anterior chamber.

It is another object of the present invention to disclose the method as described above, additionally comprising a step of configuring said implant for ophthalmic applications.

It is another object of the present invention to disclose the method as described above, additionally comprising a step of selecting said implant to be a retinal implant.

It is another object of the present invention to disclose the method as described above, additionally comprising a step of selecting said implant to be a corneal implant.

It is another object of the present invention to disclose the method as described above, additionally comprising a step of selecting said implant comprising a member of a group consisting of retinal pigment epithelium cells, photo-receptor cells and any combination thereof.

It is another object of the present invention to disclose the method as described above, additionally comprising a step of selecting said implant comprising a member of a group consisting of collagen, hydrogel, a medication and any combination thereof.

It is another object of the present invention to disclose the method as described above, wherein said medication is a slow-release medication.

It is another object of the present invention to disclose the method as described above, additionally comprising a step of said implant diameter is in a range of 0.5 mm to 8 mm.

It is another object of the present invention to disclose the method as described above, additionally comprising a step of said implant thickness is in a range of 2 μm to 500 μm.

It is another object of the present invention to disclose the method as described above, additionally comprising a step of said implant length is in a range of 0.5 mm to 8 mm.

It is another object of the present invention to disclose the method as described above, additionally comprising a step of said implant width is in a range of 0.5 mm to 8 mm.

It is another object of the present invention to disclose the method as described above, additionally comprising a step of providing a cannula.

It is another object of the present invention to disclose the method as described above, additionally comprising a step of selecting said cannula to have a diameter in a range of 0.7 mm to 4 mm.

It is another object of the present invention to disclose the method as described above, additionally comprising a step of inserting said cannula through a trocar.

It is another object of the present invention to disclose the method as described above, additionally comprising a step of inserting a trocar through said incision in said eye.

It is another object of the present invention to disclose the method as described above, additionally comprising a step of selecting the trocar diameter to be no larger than 20 G (0.9 mm).

It is another object of the present invention to disclose the method as described above, wherein the trocar diameter is larger than 20 G (0.9 mm).

It is another object of the present invention to disclose the method as described above, additionally comprising a step of effectuating actuation of removal of said implant from said implant carrier is by a mechanism comprising a member of a group consisting of a slider, a pin, a converter from circular motion to linear motion, a motor and gear, a motor and cam, a wheel, a knob, a screw, and any combination thereof.

It is another object of the present invention to disclose the method as described above, additionally comprising a step of providing a protective seal at a distal end of said insertion tool.

It is another object of the present invention to disclose the method as described above, additionally comprising a step of selecting said protective seal from a group consisting of a cap, a diaphragm and any combination thereof.

It is another object of the present invention to disclose the method as described above, additionally comprising a step of said protective seal allowing at least one of a group consisting of diffusion of gas therethrough and a nutrient path therethrough.

It is another object of the present invention to disclose the method as described above, additionally comprising a step of fluid flow performing a member of a group consisting of detaching said implant from said implant carrier, positioning said implant, transferring liquid, maintaining viability of said implant, maintaining intraocular pressure, maintaining retinal bleb size, and any combination thereof.

It is another object of the present invention to disclose the method as described above, additionally comprising steps of emplacing said implant within said insertion tool prior to shipment and of maintaining said implant in a usable condition between said emplacing and use.

It is another object of the present invention to disclose the method as described above, additionally comprising a step of maintaining said implant in a temperature range of 4° C. to 25° C. between said emplacing and said use.

It is another object of the present invention to disclose the method as described above, additionally comprising a step of maintaining said implant in a temperature range of 30° C. to 38° C. between said emplacing within said insertion tool and said use.

It is another object of the present invention to disclose the method as described above, additionally comprising a step of maintaining said implant in solution between said emplacing within said insertion tool and said use.

It is another object of the present invention to disclose the method as described above, additionally comprising a step of selecting said fluid comprising a member of a group consisting of balanced saline solution (BSS), BSS Plus, BSS Plus with additional viscous content such as Hyaluronic acid, and a CO₂ independent medium.

It is another object of the present invention to disclose the method as described above, additionally comprising a step of selecting a shape of a portion of said body from a group consisting of bent, controllably bendable, straight and any combination thereof.

It is another object of the present invention to disclose the method as described above, additionally comprising a step of passing said bent or controllably bendable portion through at least one of a trocar and a cannula.

It is another object of the present invention to disclose the method as described above, additionally comprising a step of selecting, in said bent configuration, an angle between a longitudinal axis of said body and a longitudinal axis of said bent portion to be in a range from 0° to 90°.

It is another object of the present invention to disclose the method as described above, additionally comprising a step of, in said bent configuration, an angle between a longitudinal axis of said body and a longitudinal axis of said bent portion to be in a range from 20° to 90°.

It is another object of the present invention to disclose the method as described above, additionally comprising a step of selecting, in said bent configuration, an angle between a longitudinal axis of said body and a longitudinal axis of said bent portion to be in a range from 10° to 50°.

It is another object of the present invention to disclose the method as described above, additionally comprising a step of selecting, in said bent configuration, an angle between a longitudinal axis of said body and a longitudinal axis of said bent portion to be 30°.

It is another object of the present invention to disclose the method as described above, additionally comprising a step of selecting at least a portion of a member of a group consisting of said body, said cap, said implant carrier and any combination thereof to be transparent.

It is another object of the present invention to disclose the method as described above, additionally comprising a step of said transparency enabling visibility of at least a portion of a member of a group consisting of said implant, said implant carrier and any combination thereof.

It is another object of the present invention to disclose the method as described above, additionally comprising a step of coloring at least a portion of said insertion tool to improve visibility of a member of a group consisting of said implant, at least a portion of a component of said insertion tool and any combination thereof.

It is another object of the present invention to disclose the method as described above, additionally comprising a step of selecting said color from a member of a group consisting of darker than another component, lighter than another component, fluorescent, green, and any combination thereof.

It is another object of the present invention to disclose the method as described above, additionally comprising a step of control of said insertion tool is selected from a group consisting of robotic, automatic, semi-automatic, manual, vibration damping and any combination thereof.

It is another object of the present invention to disclose the method as described above, additionally comprising a step of providing a lighting mechanism configured to provide light at a distal end of said insertion tool.

It is another object of the present invention to disclose the method as described above, additionally comprising a step of selecting said lighting mechanism comprising a member of a group consisting of an LED, a fiber optic cable, and any combination thereof.

It is another object of the present invention to disclose the method as described above, additionally comprising a step of providing a camera configured to display a view of a region near a distal portion of said insertion tool.

It is another object of the present invention to disclose the method as described above, additionally comprising a step of providing a mechanism for rotating said implant while maintaining a direction of implantation.

It is another object of the present invention to disclose the method as described above, additionally comprising a step of ejecting said implant from said implant carrier.

It is another object of the present invention to disclose the method as described above, additionally comprising a step of providing said implant carrier in contact with only said implant lower side.

It is another object of the present invention to disclose the method as described above, additionally comprising a step of configuring said implant carrier to protect said implant upper side from mechanical stress and shear.

It is another object of the present invention to disclose the method as described above, additionally comprising steps of releasably connecting the implant to the implant carrier, and configuring said releasable connection, before release, to prevent a member of a group consisting of movement of said implant, folding of said implant and any combination thereof.

It is another object of the present invention to disclose the method as described above, additionally comprising a step of providing a locking mechanism.

It is another object of the present invention to disclose the method as described above, additionally comprising a step of providing said locking mechanism comprising a member of a group consisting of locking tubes comprising holes through which a fluid can be passed, an arm contactable to said implant, a wire attachable to said implant, and any combination thereof,

It is another object of the present invention to disclose the method as described above, additionally comprising a step of flowing fluid into said insertion tool from a volume near a distal end of said insertion tool.

It is another object of the present invention to disclose the method as described above, additionally comprising a step of configuring at least a portion of said insertion tool to enable implant orientation by means of rotation of said at least a portion of said insertion tool.

It is another object of the present invention to disclose the method as described above, additionally comprising a step of providing an insertion stopper, configured to allow adjustment of a distance said implant carrier extends distally from said body of said insertion tool.

It is another object of the present invention to disclose the method as described above, additionally comprising a step of extending said implant carrier from said body by at least one predetermined distance.

It is another object of the present invention to disclose the method as described above, additionally comprising a step of said at least one predetermined distance comprising a first predetermined distance and a second predetermined distance.

It is another object of the present invention to disclose the method as described above, additionally comprising a step of selecting said first predetermined distance to be 8.5 mm.

It is another object of the present invention to disclose the method as described above, additionally comprising a step of selecting said second predetermined distance to be 4 mm.

It is another object of the present invention to disclose the method as described above, additionally comprising a step of selecting said first predetermined distance to be in a range from 7 mm to 10 mm and said second predetermined distance to be in a range from 2 to 6 mm.

It is another object of the present invention to disclose the method as described above, additionally comprising a step of selecting said first predetermined distance to be in a range from 7 mm to 10 mm and said second predetermined distance to be in a range from 2 to 5.5 mm.

It is another object of the present invention to disclose the method as described above, additionally comprising a step of selecting said extending mechanism from a group consisting of a slider, a pin, a converter from circular motion to linear motion, a motor and gear, a motor and cam, and any combination thereof.

It is another object of the present invention to disclose the method as described above, additionally comprising a step of providing said carrier tube comprising a superelastic material.

It is another object of the present invention to disclose the method as described above, additionally comprising a step of providing said carrier tube comprising Nitinol.

It is another object of the present invention to disclose the method as described above, additionally comprising a step of providing said implant carrier comprising a superelastic material.

It is another object of the present invention to disclose the method as described above, additionally comprising a step of providing said implant carrier comprising Nitinol.

It is another object of the present invention to disclose the method as described above, additionally comprising a step of providing said implant carrier comprising holes.

It is another object of the present invention to disclose the method as described above, additionally comprising a step of providing said implant carrier comprising a member of a group consisting of an anchoring structure, a compliance mechanism and any combination thereof.

It is another object of the present invention to disclose the method as described above, additionally comprising a step of said anchoring structure contacting a location on said implant selected from a group consisting of a proximal portion of said implant, a central portion of said implant, a distal portion of said implant and any combination thereof

It is another object of the present invention to disclose the method as described above, additionally comprising a step of configuring said anchoring structure to remove said implant from said implant carrier by a means selected from a group consisting of pulling said implant from said implant carrier and pushing implant from said implant carrier.

It is another object of the present invention to disclose the method as described above, additionally comprising a step of releasing said implant from said implant carrier by ending contact between said holding member and said implant.

It is another object of the present invention to disclose the method as described above, additionally comprising a step of said anchoring structure comprising at least one tooth.

It is another object of the present invention to disclose the method as described above, additionally comprising a step of said anchoring structure retaining said implant on said implant carrier before use.

It is another object of the present invention to disclose the method as described above, additionally comprising a step of said anchoring structure lifting said implant off of said implant carrier, thereby allowing removal of said implant from said implant carrier.

It is another object of the present invention to disclose the method as described above, additionally comprising a step of providing a fluid inlet, said fluid inlet in fluid communication with said implant carrier.

It is another object of the present invention to disclose the method as described above, additionally comprising a step of providing a cover tube configured to retain said carrier tube and said arm in said first configuration.

It is another object of the present invention to disclose the method as described above, additionally comprising a step of said cover tube is movable from said bendable region, removal of said cover tube from said bendable region enabling transfer of said carrier tube and said arm to said second configuration.

It is another object of the present invention to disclose the method as described above, additionally comprising a step of selecting, in said closed configuration, said implant carrier to have a diameter less than that of said carrier tube.

It is another object of the present invention to disclose the method as described above, additionally comprising a step of selecting, in said open configuration, a curvature said implant carrier to be such that a distance from a bottom center of said implant carrier to a top edge of said implant carrier is a range from 0 to 1 cm.

It is another object of the present invention to disclose the method as described above, additionally comprising a step of selecting, in said open configuration, a curvature of said implant carrier to be such that a distance from a bottom center of said implant carrier to a top edge of said implant carrier is a range from 0.2 mm to 0.5 mm.

It is another object of the present invention to disclose the method as described above, additionally comprising a step of configuring said transfer of said implant carrier from said closed position to said open position to be reversible.

It is another object of the present invention to disclose the method as described above, additionally comprising a step of effectuating said transfer of said implant carrier from said closed position to said open position by a means selected from a group consisting of pulling at least a portion of said implant carrier and pushing at least a portion of said implant carrier

It is another object of the present invention to disclose the method as described above, additionally comprising a step of transferring said implant carrier from said closed configuration to said open configuration by means of wires.

It is another object of the present invention to disclose the method as described above, additionally comprising a step of comprising said wires of shape memory alloy.

It is another object of the present invention to disclose the method as described above, additionally comprising a step of comprising said wires of Nitinol.

It is another object of the present invention to disclose the method as described above, additionally comprising a step of selecting, in said open configuration, a length of said implant carrier to be in a range from 1.5 mm to 12 mm.

It is another object of the present invention to disclose the method as described above, additionally comprising a step of selecting, in said open configuration, a width of said implant carrier to be in a range from 1.5 mm to 1 cm.

It is another object of the present invention to disclose the method as described above, additionally comprising a step of configuring said implant carrier to hold a plurality of implants.

It is another object of the present invention to disclose the method as described above, additionally comprising a step of selecting said implant carrier for a plurality of implants to be a case with shelves.

It is another object of the present invention to disclose the method as described above, additionally comprising a step of selecting said plurality of implants to be in a range from 2 to 10.

It is another object of the present invention to disclose the method as described above, additionally comprising a step of moving said implant by means of wires.

It is another object of the present invention to disclose the method as described above, additionally comprising a step of attaching said wires to said implant by means of a group consisting of ears and holes in said implant.

It is another object of the present invention to disclose the method as described above, additionally comprising a step of configuring said insertion tool to perform at least one function selected from a group consisting of emplace an ophthalmic implant, emplace an ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof.

It is another object of the present invention to disclose the method as described above, additionally comprising a step of selecting said carrier arm wire to have a width in a range from 50 μm to 0.5 mm.

It is another object of the present invention to disclose the method as described above, additionally comprising a step of selecting said carrier arm wire to have a length in a range from 5 cm to 6 cm.

It is another object of the present invention to disclose the method as described above, additionally comprising a step of loading the implant into the insertion tool either at a manufacturing site or at the operation site.

It is another object of the present invention to disclose the method as described above, additionally comprising a step of providing a slow and constant flow of fluid through the insertion tool.

It is another object of the present invention to disclose the method as described above, additionally comprising a step of providing said flow of fluid in a range of 0.05 ml/min to 1 ml/min.

It is another object of the present invention to disclose the method as described above, additionally comprising a step of flowing said of fluid via said cannula.

It is another object of the present invention to disclose the method as described above, additionally comprising a step of separating said implant from a larger implant sheet by means of punching, cutting, microdissection, laser ablation and any combination thereof.

It is another object of the present invention to disclose the method as described above, additionally comprising a step of performing said separation of said implant from said larger implant sheet while the implant is resting on or located above said implant carrier.

It is another object of the present invention to disclose the method as described above, additionally comprising a step of selecting a material of said implant carrier configured to resist damage during laser ablation or microdissection processing to separate said implant from said larger implant sheet having a property selected from a group consisting of reflecting laser light, being transparent to laser light, being resistive to high temperatures, having a protection layer and any combination thereof.

It is another object of the present invention to disclose the method as described above, additionally comprising a step of selecting said material of said implant carrier configured to resist damage during cutting or punching of said implant from said larger implant sheet having a property selected from a group consisting of having a mechanical yield strain of at least 50%, having an elastic modulus of at least 5 GPa, having a Shore durometer hardness type D of at least 50 and any combination thereof.

It is another object of the present invention to disclose the method as described above, additionally comprising a step of loading said implant into said insertion tool by transforming said insertion tool to said first body configuration.

It is another object of the present invention to disclose the method as described above, additionally comprising a step of loading said implant into said insertion tool by transforming said implant carrier to said first implant carrier configuration.

It is another object of the present invention to disclose the method as described above, additionally comprising steps of connecting said insertion tool to an operating room facility, and selecting said operating room facility from a group consisting of a fluid source, a positioning tool, a power supply, a light source, and any combination thereof.

### BRIEF DESCRIPTION OF THE FIGURES

In order to better understand the invention and its implementation in practice, a plurality of embodiments will now be described, by way of non-limiting example only, with reference to the accompanying drawings, wherein:

FIG. 1A schematically illustrates an eye;

FIG. 1B schematically illustrates the layers of the retina;

FIG. 1C schematically illustrates an artificial retinal patch that comprises a scaffold, RPE and photoreceptors;

FIG. 2 schematically illustrates insertion of an implant, with the tool's distal end within the eye;

FIG. 3 schematically illustrates a tool;

FIG. 4A-D illustrates embodiments of a tool;

FIG. 5 illustrates an embodiment of a slider mechanism;

FIGS. 6A-B and 7A-C show embodiments of implant carriers;

FIGS. 8A, 8B, 8C, 8D1 and 8D2 show embodiments of anchoring structures to attach an implant to an implant carrier;

FIG. 9A schematically illustrates an embodiment of an implant carrier with L-shaped legs;

FIG. 9B-C schematically illustrates a holding position and a release position for an anchoring structure;

FIG. 10A shows an embodiment of an implant carrier comprising an anchoring mechanism and FIG. 10B shows an enlarged view of the anchoring mechanism;

FIG. 11A-B illustrates an embodiment of a mechanism for moving an implant into a bleb;

FIGS. 12A, 12B and 12C1-12C2 show embodiments to enable detachment of the implant off the implant carrier, due to fluid flow delivered from the tool's proximal end toward the implant carrier and implant;

FIG. 13A-B shows a portion of an embodiment of the device with a sliding cover tube to keep the carrier tube straight until it has entered the eye;

FIG. 14A-B shows another embodiment of an implant carrier in an open configuration;

FIG. 15A shows an image of a carrier in a tool;

FIG. 15B shows an image of a carrier with an implant in place on the carrier;

FIG. 16A shows an image of a tool in a bleb;

FIG. 16B shows implants as implanted;

FIG. 17A shows a schematic of an exemplary embodiment of a loading tool;

FIG. 17B shows a schematic of an embodiment of a loading box;

FIG. 17C shows a schematic of an implant in a loading box being loaded onto an implant carrier;

FIG. 18 shows an embodiment of a flow chart for loading an implant into a tool;

FIG. 19 shows an embodiment of a flow chart for inserting an implant into an eye;

FIG. 20 schematically illustrates an implant holder configured to comprise a plurality of implants;

FIG. 21 schematically illustrates an embodiment of an implant carrier for a single implant;

FIG. 22A-D schematically illustrates holding/manipulating mechanisms for implants;

FIG. 23 schematically illustrates a tool with two sliding mechanisms;

FIG. 24A-C schematically illustrates an exemplary embodiment of a safety pin mechanism, allowing implantation of the implant in two steps;

FIG. 25A-C schematically illustrates an exemplary embodiment of a spring mechanism;

FIG. 26A-C schematically illustrates an exemplary embodiment of a ball plunger mechanism; and

FIGS. 27A-C, 28A-C and 29A-C illustrate embodiments of mechanisms that can convert circular to linear motion.

FIGS. 30-36 illustrate embodiments in which fluid directing means are used.

FIGS. 37a-44 illustrate another embodiment of the present invention, in which the tool comprises a proximal part and a distal part reversibly coupled to each other.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for insertion of a retinal implant under the retina.

The terms “artificial retinal patch”, “retinal implant” and “implant” hereinafter refer to a patch insertable into an eye to restore at least partial vision.

In order to treat eye disorders comprising degeneration of the photoreceptor cells such as, but not limited to age-related macular degeneration (AMD) a retinal implant can be delivered to a portion of the retina, thereby providing, in a safe and stable manner, RPE cell implantation in the eye.

For treatment of dry AMD, the RPE cell implantation is typically into the sub-retina; other locations are possible for treatment of other conditions comprising loss of RPE cells.

The retinal implant can comprise RPE cells alone or RPE cells with photoreceptors or additional retinal cells. The retinal implant should be thin, so that it will not cause a large displacement of the retina, leading to visual distortion. The retinal implant can be soft to allow integration between the retinal layers.

In some embodiments, the implantation tool is configured to implant a corneal graft or corneal implant. The graft or the matured implant can be loaded into the device while touching only one side of the graft or implant, thus maintaining the endothelial cells' vitality during insertion into the eye. The loaded implantation tool can be inserted through a 2-3 mm incision into the anterior chamber, then the implantation tool can be opened and the implant released from the tool. The implant can be inserted and released with the cells facing posteriorly. Once the implant is injected into the anterior chamber, the tool can be retracted and ejected from the eye, and the implant can be adjusted or flattened using gentle presses on the external part of the cornea. The incision can then be sutured and, through the temporal paracentesis, an air bubble can be placed underneath the graft to hold the graft in position on the stroma. These embodiments of the implantation tool of the present invention can provide a safe delivery of the implant, where only the scaffold material is in contact with the implantation tool, the implant is protected during implantation through a relatively small cut in the cornea and controlled placing of an open graft on the bare stroma is allowed.

The tool should do a minimum amount of damage to the external eye tissue and the retina, cornea or both during insertion and the tool can not damage the implant before or during the implantation process. The positioning of the implant during insertion should be controllable, with at least the implant's (x,y) location under the retina (or within the anterior chamber, and the implant directionality—having the RPE cells' apical side facing the retina (or facing posteriorly for the cornea) and the implant's angle with respect to the surface of the retina (or the surface of the anterior chamber) within desired parameters.

In some embodiments, the implant's (x,y,z) location with respect to a known location is controllable. The known location can be, but is not limited to, a definable position with respect to an eye, a heart, a liver, or a pancreas.

Retinal implants of the type of interest typically comprise a biomaterial scaffod onto which RPE cells and photoreceptor cells are “printed” using a pulsed laser beam.

One of the biggest challenges of subretinal surgery is the delivery of a medicament to a desired location. The desired subretinal space between the RPE and photoreceptors is located above the choroid, Bruch membranes and RPE cells and below the neuronal retina, vitreous and the sclera. Approaching the sub retinal space via the choroid is not possible since it will cause significant bleeding, as the choroid is composed almost entirely of blood vessels delivering nutrients and removing waste from the eye. Therefore, delivery to the sub retinal space is performed by penetrating the sclera, removing the vitreous and making a cut, as small as possible, in the neural retina (retinotomy). Although this procedure causes death of cells in the area of the retinotomy, it is still the safest approach.

The present invention discloses a tool for implanting a retinal patch into a space within the eyeball and below the surface of the retina of an eye. The tool comprises an implant carrier on which an implant rests. Before use, the implant carrier is in a closed or folded configuration and rests inside a carrier tube, enabling passage of the carrier tube through a trocar for entrance into an eye. In some embodiments the tool comprises at least one mechanism to extend the implant carrier from the carrier tube. In some embodiments, the implant carrier will gradually open sideways as extended until it reaches a spread configuration. Sideways opening of the implant carrier can be automatic, manual (controlled by a user) and any combination thereof. The tool can further comprise at least one mechanism to detach the implant from the carrier at a predetermined position in the subretina.

In some embodiments, the carrier tube is straight. In other embodiments, the carrier tube comprises a bendable portion so that the implant carrier (and the implant) enter the retina at a predetermined angle between a perpendicular to the retina and the bendable portion, typically about 30°.

The predetermined angle can be in a range from 0° to 90°. In some embodiments, it is in a range from 20° to 90°. In some embodiments, it is in a range from 20° to 40°.

Therefore, the angle between the distal portion of the carrier tube, distal to the bendable portion and the proximal portion of the carrier tube, proximal to the bendable portion, is typically 150° and can be in a range from as wide as 90° to 180°; as wide as 90° to 160°, or as narrow as from 150° to 180°.

The carrier preferably comprises Nitinol, a superelastic material with shape memory, and preferably of medical grade materials. The tool can comprise Nitinol, polyimide (Kapton), stainless steel and any combination thereof.

The implant carrier preferably comprises holes to reduce the force needed to manipulate and/or flex the implant carrier into the carrier tube and to allow easy placement of an implant on the carrier. The holes also help the implant sit on the carrier in the desired position. The holes can also allow fluid movement between the lower side of an implant and the carrier, to allow implant release. For example, fluid can drain away from the underside of an implant, or fluid can flow in between an implant and a carrier or holder, for example, to float the implant off of the carrier or holder.

The holes can be of any desired shape. Non-limiting examples include circular, oval, elliptic, elongated and cruciform.

The tool can be configured to, for non-limiting example, emplace an ophthalmic implant, emplace another ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof. In general, the device can be used whenever a small item needs to be emplaced in a precisely defined position.

Movement of the components, as described below, can be manual, by means such as, but not limited to, a finger control or a pedal, or the movement can be mechanically controlled by one or more motors.

The tool preferably comprises a fluid inlet so that fluids can pass through it, for non-limiting example, to remove the implant from the tool (either alone or as assistance to a removal tool), to transfer the implant to the implantation site, (typically the subretina), to orient the implant at the implantation site, to maintain the subretinal bleb shape and size and any combination thereof.

The implant can be soft and bendable, so that it can be curved to fit inside the carrier tube while resting on the implant carrier.

The tool allows delivery of the implant to the subretina via a small retinotomy, typically 1-1.5 mm in diameter, which is smaller than the retinotomies typical of much of the prior art.

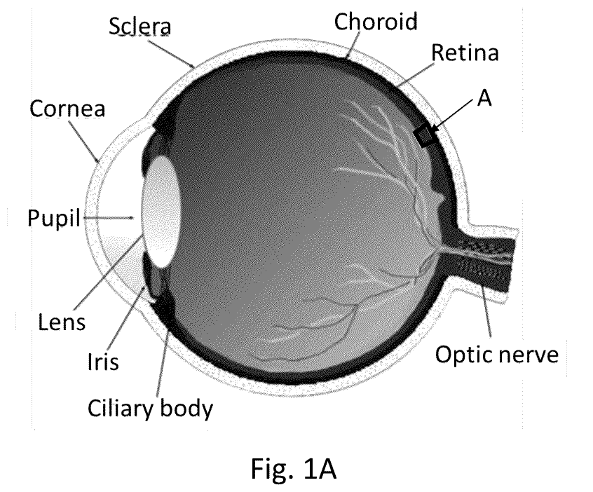

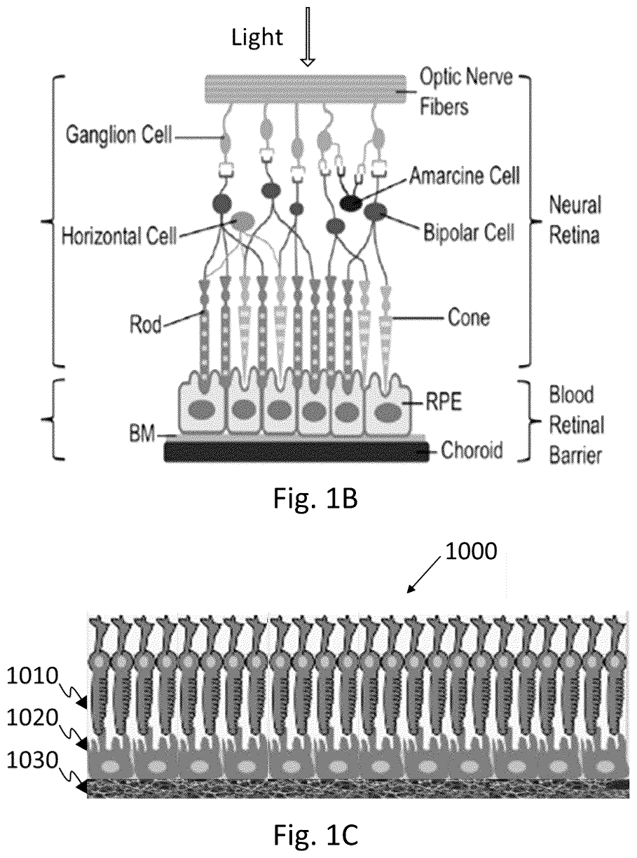

For illustrative purposes, FIG. 1A shows an exemplary eye, while FIG. 1B schematically illustrates the layers of a retina, taken from the box “A” in FIG. 1A. As shown in FIG. 1A, the eye comprises a sclera and choroid; covering the exterior of the eye. At the front of the eye are the light-gathering portions, the cornea and lens, with the pupil being a hole in the center of the iris; the iris being supported by the ciliary body. At the rear of the eye is the retina, which connects to the optic nerve. An enlarged view of the area “A” of the retina is shown in FIG. 1B. Light (entering from the top) passes through all retinal nerve cells to the photoreceptors, the rods and cones, which convert the light to neural signals. The neural signal pass from the photoreceptors to the bipolar cells and further through the other neuronal retinal nerve cells to the ganglion cells that convey the visual information to the brain. The most posterior part of the eye is aligned by retinal pigment epithelium (RPE) and Bruch's membrane (BM) which form the blood retinal barrier. The RPE cells function to support the neuronal retinal cells by supplying nutrients and growth factors, modulating blood supply to the eye and protecting the retina from the immune system. RPE cells, as part of the visual cycle, recycle the light-absorbing pigment of the photoreceptors required for vison (not shown; see FIG. 1A).

FIG. 1C schematically illustrates an artificial retinal patch (1000), a retinal implant, comprising a scaffolding layer (1030), which supports an RPE cell layer (1020) and a photoreceptor cell layer (1010). The RPE cell layer (1020) and a photoreceptor cell layer (1010) can be produced by pulsed laser beam printing, the RPE cells and the photoreceptor cells being in droplets comprising a bioink that can be moved by a laser beam, allowing the pulsed laser beam to deposit the droplets on the scaffold.

The retinal implant of the present invention is configured to be placed in the subretinal space between the RPE cells and the photoreceptors. It can be 2 μm-500 μm thick and between 0.5 mm to 8 mm in length and width; it can be rectangular or circular, preferably rectangular. In some embodiments, it is 2 mm×4 mm. Preferably, it is inserted into the retina at an angle of about 30° to the surface of the retina. In order to minimize damage to the eye and the retina, an implant tool has a circular cross-section, less than 0.9 mm in diameter. The implant can be removed from the tool by injection of fluid into the tool, floating the implant into the desired location in the retina, or by pressure on the implant by an anchoring structure.

The implant can comprise RPE cells, PR cells, hydrogel, collagen, medication and any combination thereof. Typically, the medication is a slow-release medication, although it need not be.

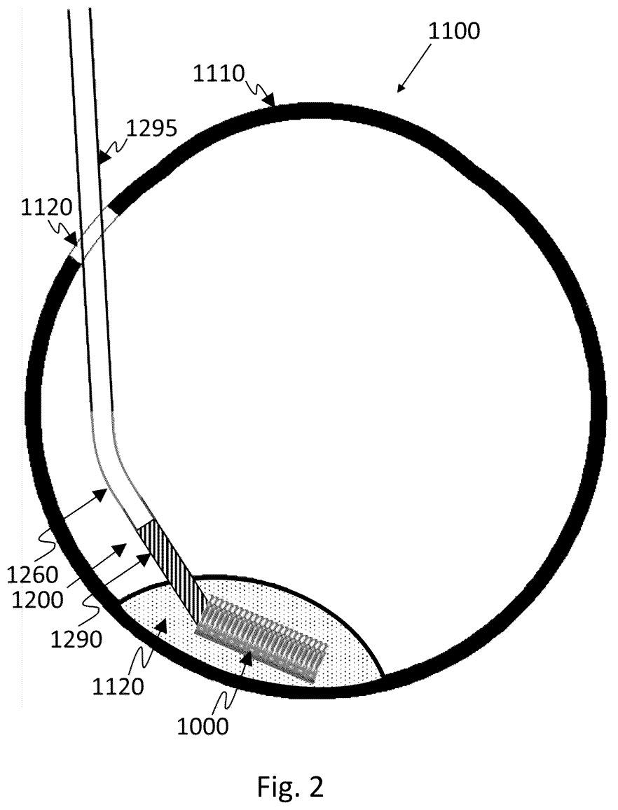

FIG. 2 schematically illustrates an exemplary tool (1200) in position to insert an implant (1000) in an eye (1110). The tool (1200) is inserted into the eye via a trocar (1295) and comprises a carrier tube (1260) and an implant carrier (1290), both of which will be discussed in more detail below. In the example shown, the implant (1000) is being placed within a bleb (1120) in the retina.

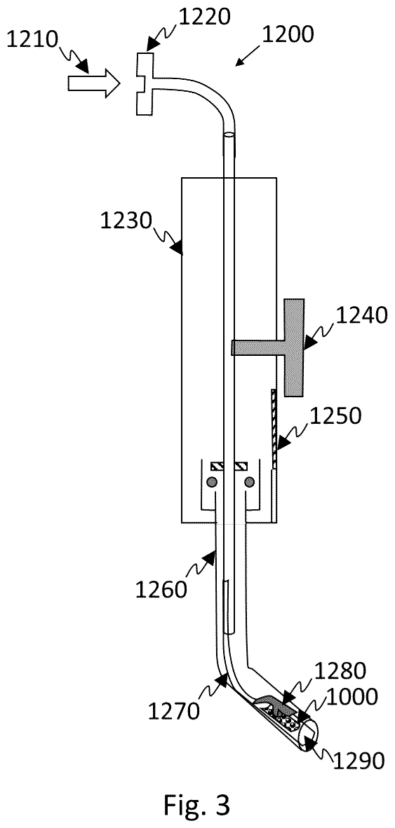

FIG. 3 schematically illustrates an embodiment of the tool (1200) in more detail. Fluid (1210) enters the tool via an inlet (1220). The fluid can displace the retinal implant (not shown), ensure that no gas enters the retina, and any combination thereof. The main body (1230), in the embodiment shown, comprises a slider (1240) to move the anchoring structure (1280) and displace the retinal implant (1000) from the implant carrier (1290). Other means of displacing the retinal implant from the implant carrier (1290) are discussed hereinbelow. The tool (1200) comprises at least one seal to prevent fluid within the eye from coming into contact with a hand or other manipulator of the tool, for non-limiting example, a robotic gripper.

In the embodiment shown, movement of the slider (1240) is limited by a slot limiter (1250); longer displacements of the slider (1240) can occur when the top of the slot limiter (1250) is further down the main body (1230). A carrier tube (1260) conveys the fluid from the open distal end of the tool (1200) and, within the carrier tube (1260) are the implant carrier (1290) with an arm (1270) extending into the carrier tube (1260) and an anchoring structure (1280) preventing the implant from sliding out unintentionally (1000) from the implant carrier (1290).

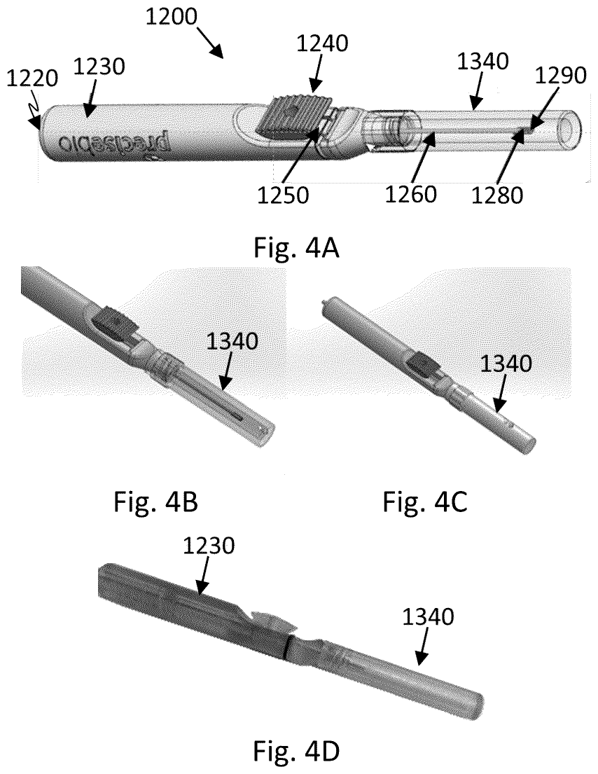

FIG. 4A illustrates an embodiment of the tool (1200). The tool has a fluid inlet (1220) at the proximal end of the body (1230). The fluid inlet (1220) can be covered, capped or sealed before use to maintain sterility. The tool (1200) comprises a slider (1240) and a limiter (1250) to extend the anchoring structure (not shown) when the implant (not shown) is to be emplaced. A carrier tube (1260) (in the size of 20 G or less, or more than 20 G) extends distally from the body (1230). Within carrier tube (1260) are an implant carrier (1290) and an anchoring structure (1280) preventing the implant from sliding out unintentionally (1000) from the implant carrier (1290). Preferably, before use, the carrier tube (1260) is contained within a protective cap (1340). Preferably, the protective cap (1340) is filled with fluid such as balanced saline solution (BSS), BSS Plus or BSS Plus with additional viscous content such as Hyaluronic acid, the Hyaluronic acid being in a range from 0.05% to 0.5%.

FIGS. 4B and 4C illustrate embodiments of protective caps (1340), the protective cap (1340) of FIG. 4B being transparent and the protective cap (1340) of FIG. 4C being opaque. Some embodiments have a translucent protective cap; an embodiment of the protective cap can comprise a member of a group consisting of transparent, translucent, opaque and any combination thereof. A protective cap (1340) that is at least partly transparent enables a user to check the usability of the device, while an opaque protective cap (1340) can protect the contents—especially the implant—from degradation caused by light.

The embodiment of FIG. 4D has both a transparent body (1230) and a transparent protective cap (1340).

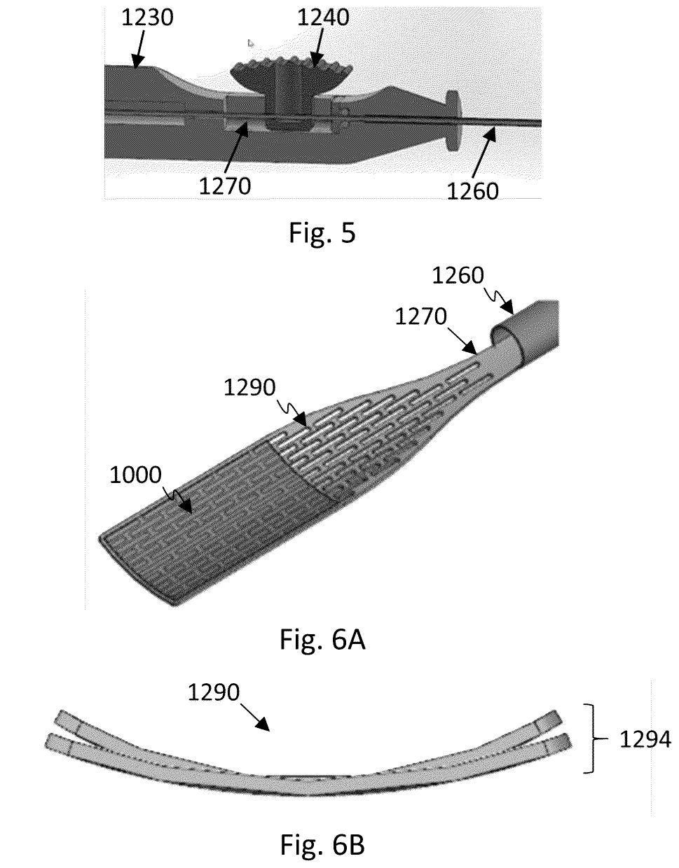

FIG. 5 illustrates an embodiment of a body (1230) with a slider (1240). The slider (1240) is connected to a carrier arm (1270), the carrier arm (1270) configured to slide distally within the carrier tube (1260) when the slider (1240) is slid distally.

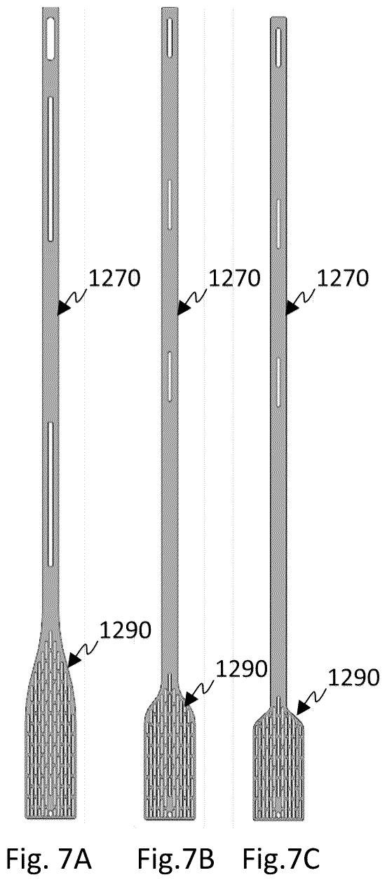

FIGS. 6A-B and 7A-C show embodiments of carrier arms (1270) and implant carriers (1290). FIG. 6A shows an embodiment with a bendable Nitinol carrier tube (1260), which holds the carrier arm (1270), with the implant carrier (1290) distal to the carrier tube (1260). In some embodiments, the carrier tube (1260) is not bendable. In some variants of a non-bendable carrier tube (1260), the carrier tube (1260) is straight; in other variants of a non-bendable carrier tube (1260), it has a fixed bend radius.

In the embodiment of FIGS. 6A-B, the implant carrier (1290) is mesh-like, A mesh-like implant carrier (1290) can prevent the implant (1000) from clinging to the implant carrier (1290), it can allow the implant carrier (1290) to flex more easily, so that it will more easily fit into the carrier tube (1260), can allow an implant (1000) to be more easily picked up by the implant carrier (1290) and any combination thereof.

Typically, a Nitinol carrier tube (1260) wall is 50 μm thick; wall thickness can be in a range from 20 μm to 100 μm. The carrier tube (1260) diameter is designed to allow for insertion through trocar (not shown). The wall thickness will depend on the carrier wall material. For example, the wall thickness for a stainless steel carrier tube (1260) is 50 μm, with the wall thickness being in a range from 20 μm to 100 μm.

FIG. 6B shows the distal end of an implant carrier (1290). In the embodiment shown, the implant carrier (1290) and the carrier arm (1270) are curved. This helps the implant carrier (1290) to fit into a Nitinol carrier tube (1260). In the case of an ophthalmic implant, the depth of the curvature (1294), from the bottom of the center of the implant carrier (1290) to the upper edge of the highest point of the implant carrier (1290), can be in a range from 0 to 1 mm preferably in a range from 0.2 mm to 0.5 mm.

According to another embodiment, the carrier (1290) is substantially curved when the same is outside the carrier tube (1260) and not flat, such that when the implant (graft) is placed on the same, it will assume its curvature (even before entrance into the carrier tube (1260). Obviously, when inside the carrier tube (1260), the carrier (1290) and the implant will assume a rolled configuration, as detailed above.

According to some embodiment, the radius of curvature of the carrier (1290) is in the range of 0.25-10 mm; preferably, in the range of 1.0-3.0 mm.

According to another embodiment, the carrier tube (1260) is characterized by radius in the range of 0.1-4 mm; preferably, in the range of 0.1-0.7 mm.

Such curvature of the carrier (1290) is highly important and crucial for the successful insertion of the carrier (1290) and the substrate/implant/graft (1000) into the carrier tube (1260). It would be appreciated that such curvature is utilized to facilitate insertion of the carrier (1290) and the substrate/implant/graft (1000) into the carrier tube (1260) and thus, taking a shape selected from a group consisting of folded, rolled and bent.

When comparing insertion of the carrier (1290) and the substrate/implant/graft (1000) thereupon into the carrier tube (1260) when (a) the initial shape of the carrier (1290) is flat; to (b) an initial shape of the carrier (1290) being a curved shape, it is found that insertion of an already curved shape of the carrier (1290) is much easier and results in a smooth retraction into the carrier tube (1260), without any damages to the implant. On the contrary, when the carrier (1290) and the substrate/implant/graft (1000) thereupon is initially flat (or straight), insertion of the same into the carrier tube (1260) is impossible without any damage to the implant.

Furthermore, such initial curvature of the carrier (1290) (when the carrier (1290) and the implant/graft (1000) are outside the carrier tube (1260)) is important for preventing the implant/graft (1000) from ‘slipping’ from the sides of the carrier (1290).

Yet more, such initial curvature facilitate maintenance of the desired orientation of the implant/graft (1000) and prevents miss orientation of the same.

Lastly, such initial curvature also facilitates the insertion of the carrier (1290) back into the carrier tube (1260) after the implant/graft (1000) is placed into its position in the eye.

FIG. 7A-C shows embodiments of implant carriers (1290). Implant carriers (1290) can be different lengths to carry different sizes of implants or to deliver implants more efficiently. FIG. 7A shows a long implant carrier (1290), FIG. 7B shows a mid-length implant carrier (1290), and FIG. 7C shows a short implant carrier (1290). In this embodiment, the total length of the implant carrier (1290) plus carrier arm (1270) is fixed; the longer implant carriers (1290) having shorter carrier arms (1270) and vice versa. In some embodiments, the carrier arm (1270) length remains constant, so that the total length depends on the length of the implant carrier (1290). In some embodiments, both the implant carrier (1290) length and the carrier arm (1270) lengths differ.

The length of the implant carrier (1290) can be in a range from 1.5 mm to 15 mm.

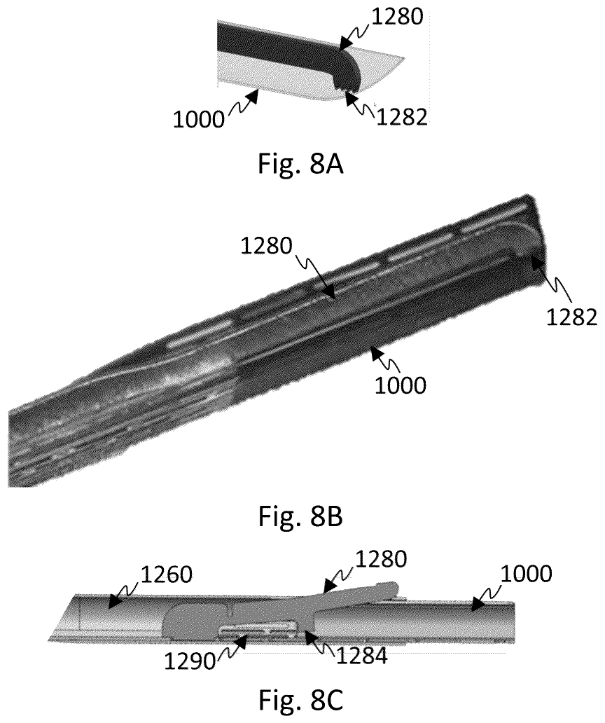

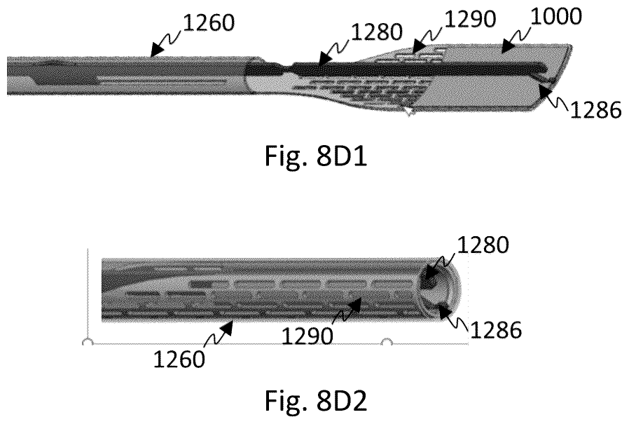

FIGS. 8A, 8B, 8C, 8D1, 8D2, 8E1 and 8E2 show embodiments of anchoring structures to attach an implant (1000) to an implant carrier (1290). FIG. 8A-8B show an embodiment of an anchoring structure (1280) with teeth (1282) to grip the implant (1000) at the front of the implant. In some embodiments, the teeth (1282) prevent the implant (1000) from moving before use, with the anchoring structure (1280) lifting from the implant (1000) at implantation time. In such embodiments, fluid is used to transport the implant (1000) to the implantation site. In some embodiments, the teeth (1282) prevent the implant (1000) from moving before use, and the anchoring structure moves forwardly at implantation time to transport the implant (1000) off the carrier (1290). In FIG. 8B, the implant carrier (1290) is partly outside the carrier tube (1260); the anchoring structure has lifted off the implant, releasing the projection (1286) from the implant and allowing the implant to be removed from the carrier. FIG. 8C shows an embodiment of an anchoring structure (1280) with a projection (1284) which contacts the rear of the implant (1000), so that it can hold the implant (1000). FIGS. 8D1 and 8D2 show an embodiment of an anchoring structure (1280) with a projection (1286) which contacts the front of the implant (1000), so that it can pull the implant (1000) off the implant carrier (1290). FIG. 8D1 shows the device in an open position, with the anchoring structure (1280), implant carrier (1290) and implant (1000) outside the carrier tube (1260), while FIG. 8D2 shows the device in a closed position, with the anchoring structure (1280), implant carrier (1290) and implant (1000) inside the carrier tube (1260).

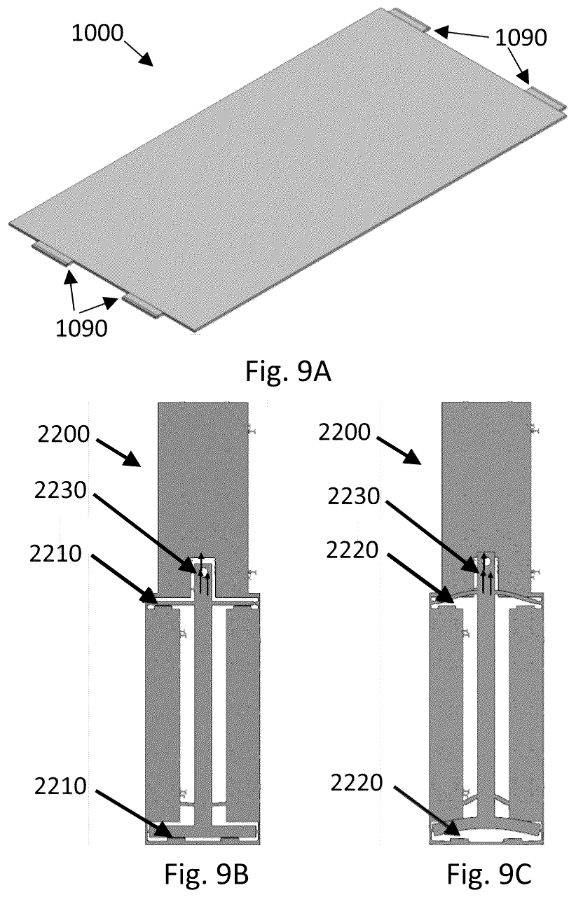

FIGS. 9A-C schematically illustrate an embodiment of an implant (1000) and implant carrier (2200) where release of the implant (1000) from the implant carrier (2200) is by means of compliance.

FIG. 9A schematically illustrates the implant (1000), which has L-shaped legs (1090), where the outer, vertical portion of the legs is configured to fit into a gap in the implant carrier (2200), thereby holding the implant (1000) in place on the implant carrier (2200).

FIGS. 9B and 9C schematically illustrate an embodiment of a holding (2210) and a release (2220) position for an implant carrier (2200) with a compliance mechanism, functioning through elastic body deformation. In the holding (2210) position, the bars (2230, 2240) of the implant carrier (2200) are straight and horizontal as shown in FIG. 9B. The vertical part of each L-shaped leg fits snugly into the gap between a straight bar (2230, 2240) and an adjacent part of the implant carrier (2200), thereby holding the implant (1000) in position.

In the release (2220) position, the bars (2230, 2240) of the implant carrier (2200) are bowed; as shown in FIG. 9C. By bowing the bars (2230, 2240) of the implant carrier, the gap (2220) between the bars (2230, 2240) and the remainder of the implant carrier is enlarged, freeing the implant (1000) from the implant carrier (2200). The implant (1000) can then be transferred to the implant site by fluid flow, any other of the transfer means disclosed herein, or any convention means of moving an implant (1000).

Transfer from the straight (2210) to the bowed (2220) positions is accomplished by pulling (arrows, 2230) on the center of the implant carrier (2200).

Since there are no hinges and the implant carrier (2200) is made from a single part, the rigidity and flexibility of the release mechanism relies on the compliance, the elastic deformation, of the implant carrier material.

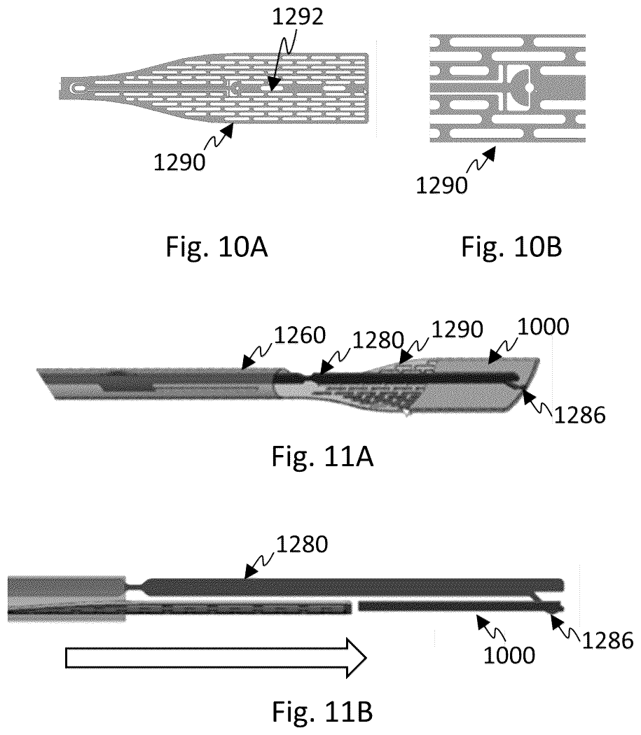

FIG. 10A-B shows an embodiment of an implant carrier (1290) comprising another anchoring mechanism (1280), where FIG. 10A shows the implant carrier (1290) and FIG. 10B shows an enlargement of the anchoring mechanism in the implant carrier (1290). In the embodiment of FIG. 10A-B the implant carrier (1290) has a built-in compliance mechanism which can physically hold the implant while pressing a small portion of the implant through a narrow gap (30-50% of the implant thickness). To release the implant, the rear slot (1292, in the middle of the carrier) can be pulled via a suture, Nitinol wire, or stainless-steel wire to the proximal end of the implant carrier. When the suture or the wire is being pulled, the width of the narrow gap increases to about 100-200% of the implant thickness, thus allowing the implant to be released via any desired method (fluid, air etc.). It should be noted that combinations of the embodiments of FIGS. 8A, 8B, 8C, 8D1, 8D2, 8E1, 8E2 and 9A-B are possible.

FIG. 11A-B shows use of the embodiment of the anchoring structure (1280) as shown in FIG. 8D1-8D2. FIG. 11A shows the anchoring structure (1280) with its projection (1286) contacting the front of the implant (1000). The implant (1000) is resting on the implant carrier (1290), which is outside the carrier tube (1260) in the open configuration. FIG. 11B shows the embodiment after the implant carrier (1290) is fully extended from the carrier tube (1260) and after the anchoring structure (1280) has released the implant (1000). The direction of movement (arrow) is to the right. The anchoring structure (1280) can release the implant (1000) by lifting off of it, after which fluid flow can remove the implant (1000) from the implant carrier (1290), or the anchoring structure (1280) can pull or push the implant (1000) off the implant carrier (1290).

In some embodiments, a closed tube is used. In other embodiments, it is an open tube.

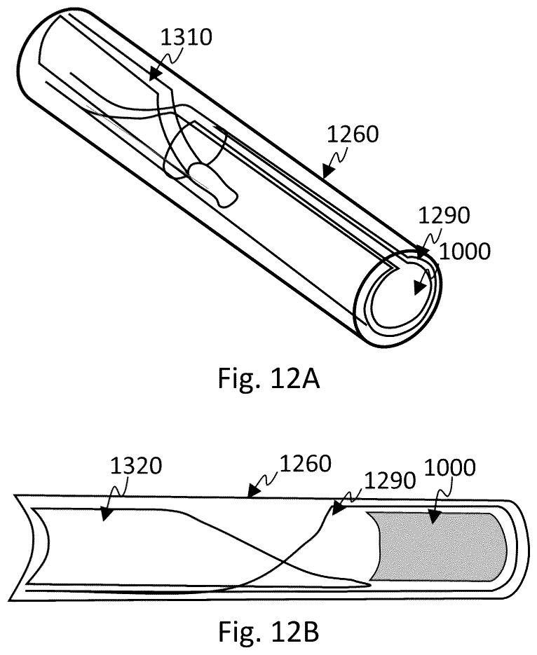

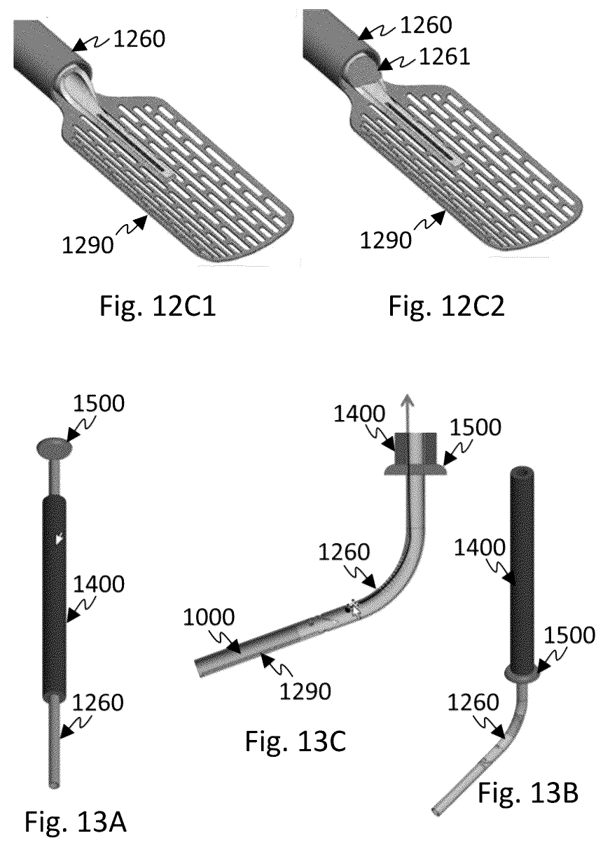

FIG. 12A-C shows embodiments of devices to detach an implant from an implant carrier. In FIG. 12A, a cannula (1310) is positioned in the carrier tube (1260), under the implant (1000) and above the implant carrier (1290), elevating the implant and allowing detachment from the carrier. In some embodiments, additional fluid flowing through the cannula (1310) will float the implant (1000) off the implant carrier (1290) and carry the implant (1000) into the subretina.

In FIG. 12B, a closed tube (1320) is used as a cannula. The closed tube (1320) is positioned in the carrier tube (1260), under the implant (1000) and above the implant carrier (1290). Fluid flowing through the closed tube (1320) will float the implant (1000) off the implant carrier (1290) and carry the implant (1000) into the subretina. Since the tube (1320) is closed, it will elevate the implant, making it easier for the solution to carry implant (1000) off of the implant carrier (1290) and into the subretina.

FIG. 12C1-2 illustrates an embodiment showing fluid flow from a closed tube (1320). The closed tube (1320) is positioned in the carrier tube (1260) and above the implant carrier (1290). In FIG. 12C1, the fluid is not yet flowing; FIG. 12C2 shows fluid flow (1261) that has reached the end of the closed tube (1320) and is about to flow onto the implant carrier (1290).

In some embodiments, an open cannula is used. In some embodiments, the cannula is pushed out from the carrier tube together with the implant carrier, to release the implant from an already opened carrier. The cannula typically has an outer diameter in a range from 0.2 mm to 2 mm. In some embodiments, the cannula is insertable into the eye or other body part through a trocar. Typically, the ophthalmic trocar diameter is no larger than 20 G (0.9 mm); however larger diameter are also within the scope of the present invention.

In some embodiments, the implant carrier and fluid cannula can be made from a single polyimide tube. In some embodiments, the implant carrier and fluid cannula are separate components, both made of polyimide. In some embodiments, the carrier is polyimide and the fluid cannula is metal such as Nitinol or stainless steel, in yet other embodiments, the carrier is metal and the fluid cannula polyimide.

In some embodiments, in order to allow an easily-controllable entry of the tool into the eye, the tool needs to remain straight on entry. However, at least a portion of the tool, near the distal end thereof, needs to bend to an angle of about 30° in order to enter the subretina at the correct angle, so that the implant enters the retina at the correct angle for maximum efficacy in emplacement. Therefore, in preferred embodiments, the tool comprises, in addition to the bendable portion of the carrier tube, a cover tube that covers the bendable portion and holds it straight until the bendable portion has entered the eye and, preferably, until the distal end of the carrier tube is nearing the rear of the eye. When the tool distal end reaches the appropriate position in the eye, the bendable portion can be removed from the cover tube, at which time the bendable portion returns to its unstrained, bent shape.

FIG. 13A-C shows schematic illustrations of embodiments of a cover tube (1400). In FIG. 12A, the cover tube (1400) is shown in an initial position. The cover tube (1400) is below (distal to) the trocar (1500) through which the tool is inserted into the eye. The carrier tube (1260) passes through the trocar (1500) and the cover tube (1400), with the bendable portion of the carrier tube (1260) within the cover tube (1400).

FIGS. 13B-C show a schematic illustration of the embodiment a cover tube (1400), after the bendable portion of the carrier tube (1260) has exited the cover tube (1400), with FIG. 12C being an enlarged view of the distal end of the cover tube (1400) and the carrier tube (1260). To remove the cover tube (1400) from the bendable portion of the carrier tube (1260), the cover tube is moved proximally (arrow in FIG. 13C) until it is proximal to (above) the trocar (1500) and outside the eye. FIG. 13C also shows the implant (1000) at the distal end of the carrier tube (1260), with the implant carrier (1290) shown in a closed configuration.

In order to pass the implant carrier (1290) through the trocar (1500) and the cover tube (1400), the implant carrier (1290) must have a diameter smaller than the diameter of the cover tube (1400). In this closed configuration, the implant (1000) is surrounded by the implant carrier (1290). Therefore, it is preferable to have the implant carrier (1290) open before the implant (1000) is removed from the implant carrier (1290).

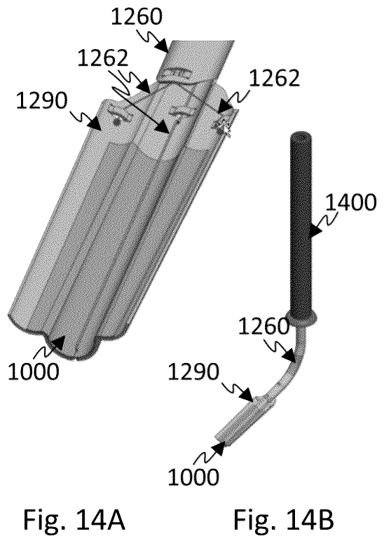

FIG. 14A-B show an exemplary schematic illustration of an implant carrier (1290) in an open configuration. FIG. 14B shows the cover tube (1400) and trocar (1500), with the carrier tube (1260) and implant carrier (1290) in the open configuration, with an implant (1000) on the implant carrier (1290). FIG. 14A is an enlarged view of the distal end of the carrier tube (1260), the open implant carrier (1290) and the open implant (1000). In this exemplary embodiment, strings or wires (1262), typically Nitinol wires, are used to pull the implant carrier (1290) to its open configuration.

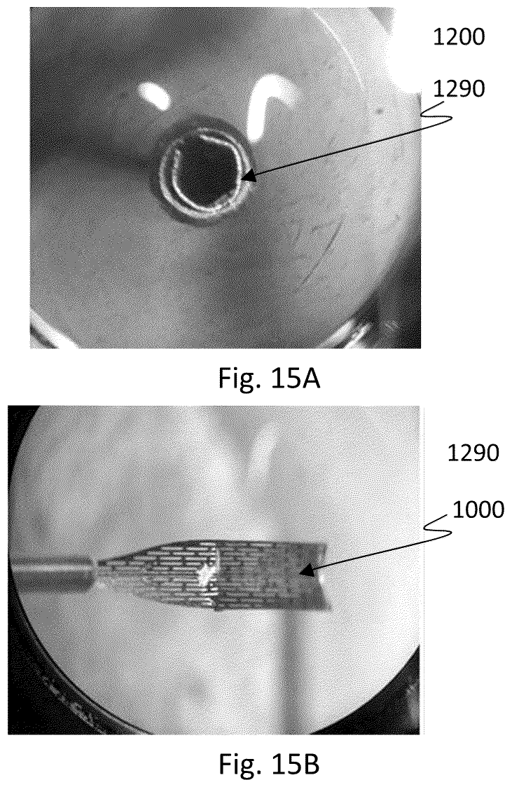

FIG. 15A shows an end view of an embodiment of a carrier (1290) inside a tool (1200), while FIG. 15B shows a view of an embodiment of a carrier (1290) with an implant (1000) on it.

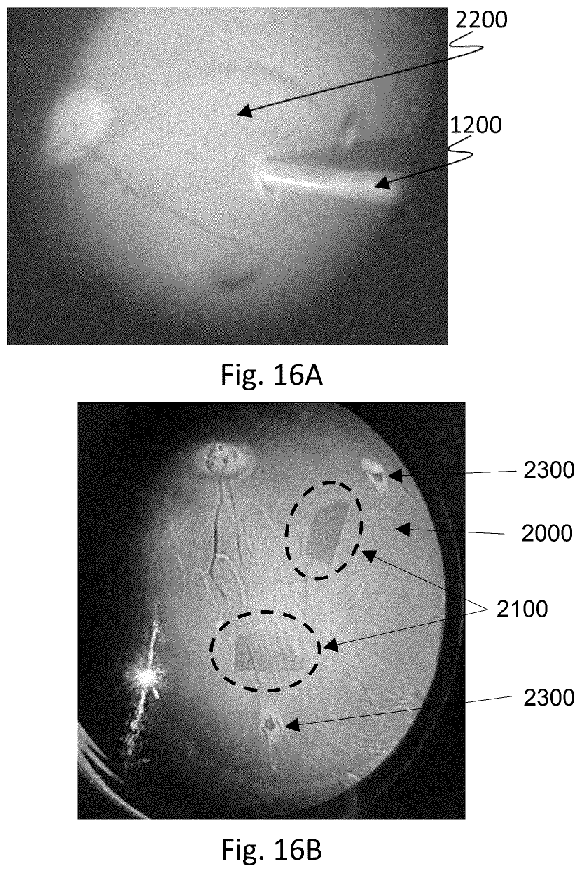

FIG. 16A shows a tool (1200) inserting an implant (not shown) into a bleb (2200), while FIG. 16B shows two implants (darker rectangles inside dashed circles 2100) as implanted using the tool of the present invention in the retina of an ex-vivo pig's eye (2000). In two sequential operations in one eye, two retinotomy holes (2300) were made, through which tools were inserted into the subretinal bleb and retinal implants were implanted. It is clear from the evenness of the visible color that the implants are at a constant depth in the retina.

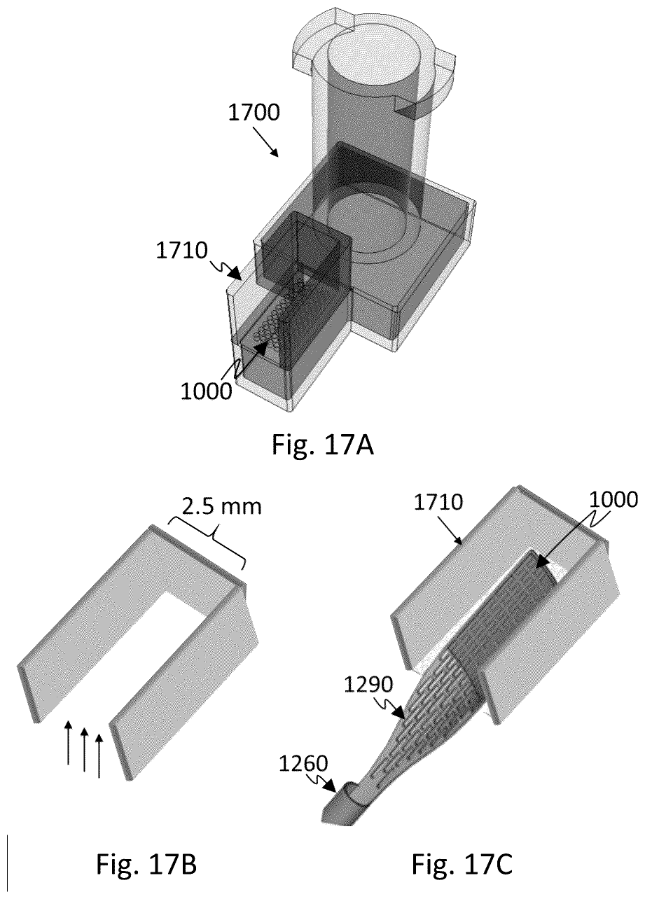

Implants must be loaded into the tool before use, preferably at the manufacturing site of the implant. FIG. 17A shows a schematic of an exemplary embodiment of a loading tool (1700). The loading box (1710) is filled at least partially with liquid so that the implants (1000) float within the loading box (1710). The loading box (1710) is configured by means of shape and size to position the implant (1000) so that it can be properly loaded onto an implant carrier. In some variants of loading tools, the implants (1000) float on a surface of a liquid; in other variants, the implants (1000) float submerged in a liquid. The liquid can be saline solution such as BSS, BSS Plus or BSS Plus with additional viscous content such as Hyaluronic acid, the Hyaluronic acid being in a range from 0.05% to 0.5%.

Typically, preparation of an ophthalmic implant comprises generating a sheet of implant material by pulsed laser beam printing, the RPE cells and the photoreceptor cells being in droplets comprising a bioink that can be moved by a laser beam, allowing the pulsed laser beam to deposit the droplets on the scaffold. In some embodiments, a sheet of implant material can be generated by pulsed laser beam printing with other materials and/or other types of material in the droplets. After preparation of the sheet of implant material, individual implants are separated from the larger implant sheet by means of punching, cutting, microdissection, laser ablation and any combination thereof.

The implants can be cut from the implant sheet and then transported to the implant carrier, or one or more implant carriers can be positioned under the implant sheet and the implant(s) cut from the implant sheet while the implant sheet is on the carrier.

In embodiments where each implant is cut from the implant sheet while overlying, resting on or situated over, an implant carrier, precautions must be taken to ensure that the implant carrier is not damaged by the cutting means used.

The precaution can be selecting a material for the implant carrier that is not damaged by the cutting means, providing a protective means between the implant carrier and the cutting means, and any combination thereof. The protective means can be a flow of fluid, a protective layer, programming to prevent contact between the cutting means and the implant carrier, any other conventional means of protection, and any combination thereof.

The material of the implant carrier can resist damage during laser or microdissection processing of an overlying implant sheet to generate an implant by (A) reflecting laser light, (B) being transparent to laser light, (C) being resistive to high temperatures, (C) having a protection layer, and any combination thereof. The material of the implant carrier can resist damage during cutting or punching of an overlying implant sheet to generate an implant by having (A) high mechanical yield strain, of at least 50%, (B) having a high elastic modulus, of at least 5 GPa, having a Shore durometer hardness type D of at least 50, and any combination thereof.

FIG. 17B shows a schematic of an embodiment of a loading box (1710). The loading box (1710) is typically about 2.5 mm wide; the range can be from 2 mm to 4 mm. In some embodiments, BSS flows upward into the loading box (1710) to make the implant float.

FIG. 17C shows a schematic of an implant (1000) in a loading box (1710) being loaded onto an implant carrier (1290). The distal end of the carrier tube (1260) can be seen. Preferably, the implant (1000) is floating within the loading box (1710) during the loading operation.

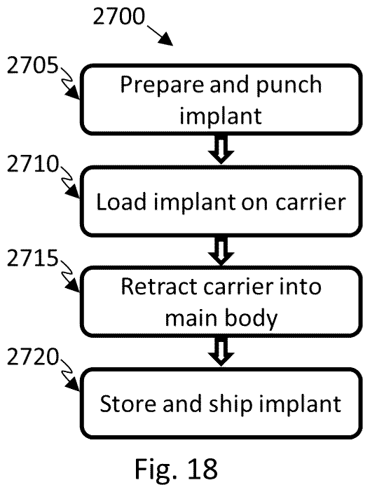

FIG. 18 shows an exemplary embodiment of a flow chart (2700) for preparation of a tool containing an implant. An implant is generated (2705) by creating a sheet of implants and punching from the implant sheet at least one implant of the desired size. The implant (or each implant, if there is more than one per tool) is loaded (2710) on the tool's carrier (or carriers, for multiple-implant tools). The tool's carrier(s) is retracted (2715) into the tool body, and the loaded tool, complete with implant(s), is shipped (2720) and stored until needed.

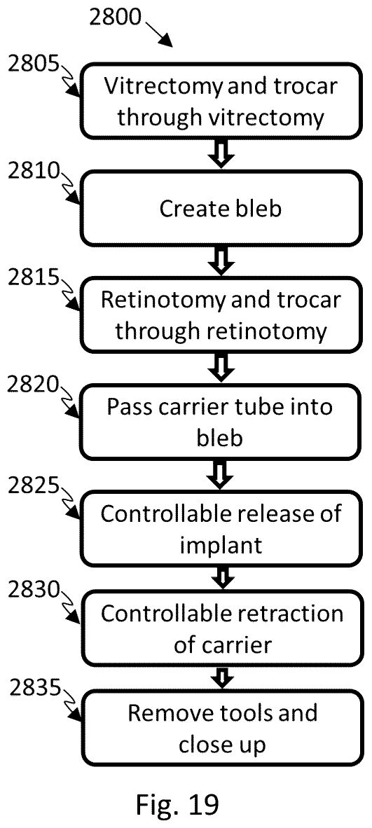

FIG. 19 shows an exemplary embodiment of a flow chart (2800) for insertion of the implant under the retina. First, the physician performs a 23 g or 25 g pars plana vitrectomy, including posterior vitreous detachment induction (if needed), (2805) Then, a syringe with, for example, a 41 gauge needle, filled with saline is used to create a retinal bleb (2810). Preferably, diathermy is used to perform a retinotomy (2815), preferably with a diameter of less than 1 mm. One of the superior trocars is replaced by a 20 G and the carrier tube with the implant is inserted through the trocar and then through the retinotomy into the subretinal space of the bleb (2820). The implant is controllably released (2825) from the carrier. Once the implant is inserted under the retina, the carrier is controllably retracted (2830) into the carrier tube and removed from the eye through the 20 g trocar. The bleb volume can be reduced by sucking fluid from the with the aid of soft tip cannula. A perfluorocarbon liquid is used to flatten the detached retina, followed by endolaser around the retinotomy site (2835). An air-fluid exchange is performed, the eye is filled with silicone oil (Perfluoropropane (C3F8) gas or any conventional means) and the sclerotomies are sutured.

The location of the retinotomy site within the bleb is important—the retinotomy should not be in front of the fovea, and also should not be too close to the bleb edge so the tool will not scratch the RPE during insertion.

FIG. 19 schematically illustrates an embodiment of an implant holder (1800) configured to comprise a plurality of implants (1000), in this example, 3 implants (1000) in a single holder (1800). The number of implants in the holder can be in a range from 2 to 10. The implant holder (1800) comprises a case (1820) containing shelves (1810), preferably of Nitinol, with implants (1000) resting on the shelves. In this example, the implant holder (1800) is 1 mm wide; the implant holder (1800) can be any size sufficiently larger than the implants (1000) that would allow an implant carrier to get between the shelves to collect the implants (1000).

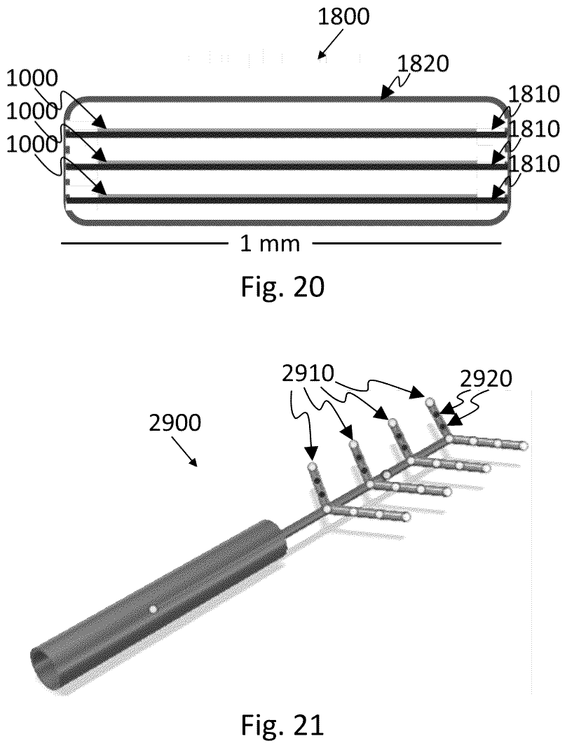

FIG. 20 schematically illustrates an embodiment of a brush-type implant carrier (2900) configured to hold an implant on brush locking tubes (2910). The brush locking tubes (2910) comprise holes (2920) through which a fluid can be pushed, pulled or both. An implant (not shown) can be held to the brush locking tubes (2910) by pulling fluid through the holes (2920), and the implant (not shown) released from the brush locking tubes (2910) by pushing fluid through the holes (2920). The brush locking tubes (2910) can easily be retracted into a carrier tube after removal of the implant from the brush locking tubes (2910), typically, after emplacement of the implant in the subretina.

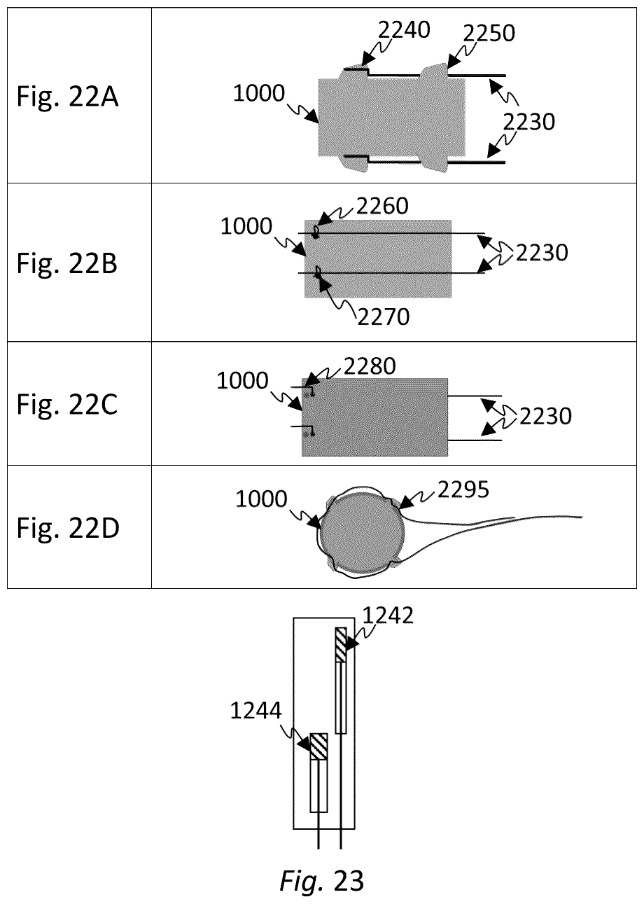

FIG. 22A-D schematically illustrates embodiments of holding (or locking) mechanisms for implants (1000), all of which allow the implants (1000) to be pushed and/or pulled into the subretina. FIG. 22A schematically illustrates an embodiment with Nitinol wires (2230) attached to one set of “ears” (2240) at the top and bottom of the implant (1000). The wires (2230) then pass under a second set (2250) of ears. FIG. 22B schematically illustrates an embodiment with Nitinol wires (2230), each wire (2230) being attached to the implant (1000) via a post (2270) and a loop (2260) of wire. Removal of the Nitinol wire (2230) frees the loop (2260) of wire from the implant (1000). FIG. 22C schematically illustrates an embodiment with Nitinol wires (2230) which pass through (2280) the implant (1000). FIG. 22D schematically illustrates an embodiment with a circular implant (1000) and a single Nitinol wire (2290) which encircles the implant (1000), passing through ears (2295) on the circumference of the implant (1000).

FIG. 23 schematically illustrates a less-preferred embodiment, which comprises two sliding mechanisms, a first sliding mechanism (1242) with a first travel distance A to extract an implant from a carrier tube, and a second sliding mechanism, with a second travel distance B to detach the implant from an implant carrier. In more-preferred embodiments, extraction and detachment are performed with a single sliding mechanism, such as is shown in, for example, FIG. 5.

In the case of a retinal implant implantation tool, the first travel distance to extract an implant from a carrier tube can be in a range from 0 to 12 mm, while the second travel distance to detach the implant from an implant carrier can be in a range from 2 mm to 6 mm. In some embodiments, the first travel distance is 8.5 mm and the second travel distance is 4 mm.

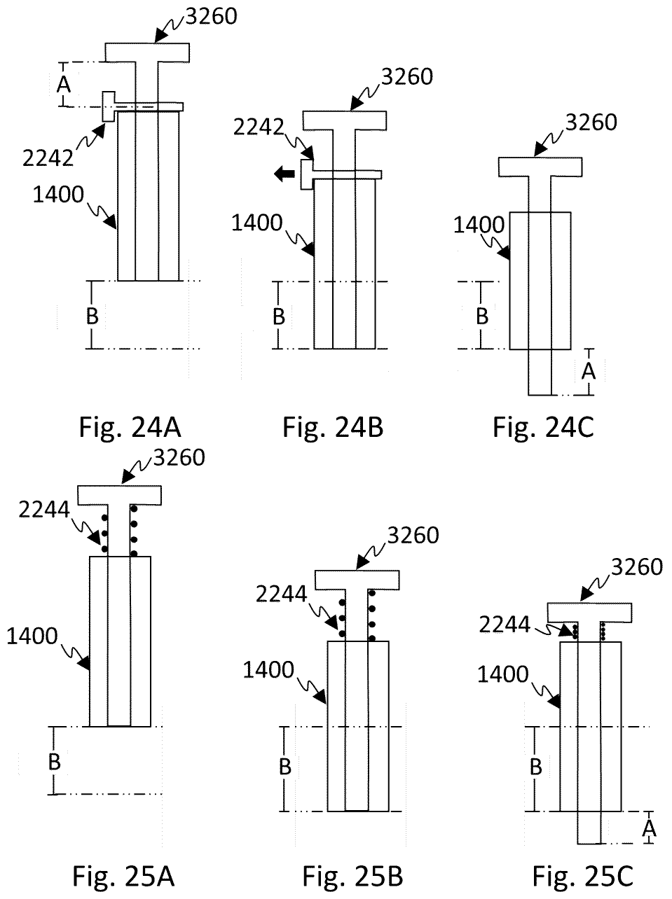

FIGS. 24A-C, 25A-C and 26A-C illustrate embodiments of safety mechanisms that allow control of which components move relative to each other, the amount they move and the order they move in, while still enabling these movements to be effectuated using a single smooth movement of an external control.

FIG. 24A-C schematically illustrates an exemplary embodiment of a safety latch (2242). In this exemplary embodiment, the safety latch (2242) is mounted at the side of the cover tube (1400). As shown in FIG. 24A, when the safety latch (2242) is in place, the cover tube (1400) and any components (3260) passing through the cover tube (1400) move as a unit. As shown in FIG. 24B, after the cover tube (1400) and inner components (3260) have moved distally (down) by a distance B, the safety latch is removed (arrow). That allows, as shown in FIG. 24C, the inner components (3260) to move distally by distance A relative to the cover tube (1400) where distance B is larger than distance A.

FIG. 25A-C schematically illustrates another exemplary embodiment of a safety mechanism. In this exemplary embodiment, the safety mechanism comprises a spring (2244) mounted between the cover tube (1400) and the components (3260) passing through the cover tube (1400). As shown in FIG. 25A, when the spring (2244) remains in an expanded condition, the cover tube (1400) and any components (3260) within the cover tube (1400) move as a unit. As shown in FIG. 25B, after the cover tube (1400) and inner components (3260) have moved distally (down) by distance B, the spring (2244) can start to contract. When the spring (2244) is fully contracted, as shown in FIG. 25C, the inner components (3260) have moved distally by distance A relative to the cover tube (1400), where distance B is larger than distance A. The spring (2244) also functions as a safety feature. When pressure on the proximal most surface of the components (3260) is released, the spring will expand, thereby causing the inner components (3260) to retract into the cover tube (1400) so that the device returns to the configuration seen in FIG. 25B.

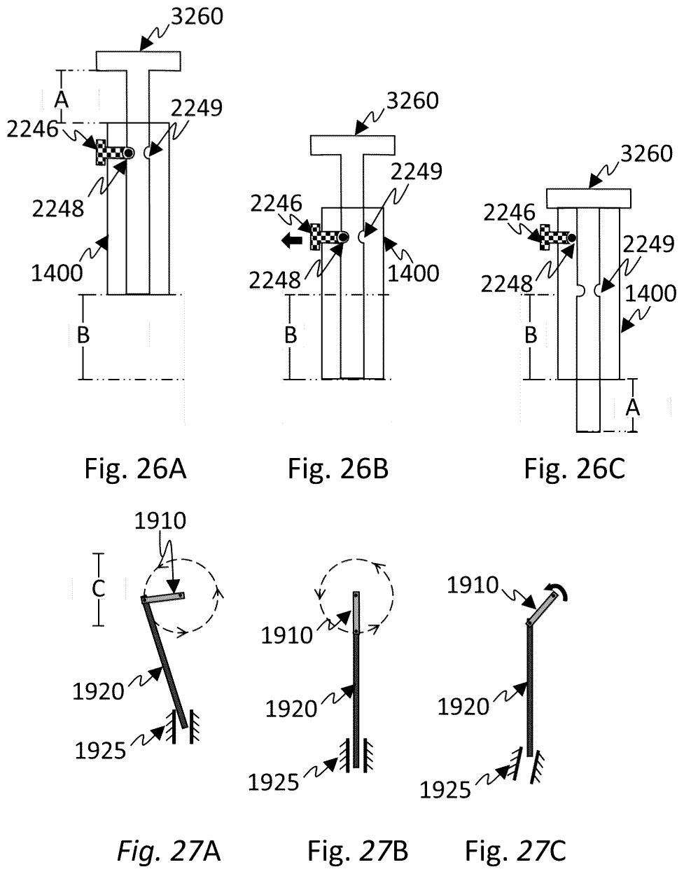

FIG. 26A-C schematically illustrates another exemplary embodiment of a safety latch (2246, 2248, 2249). In this exemplary embodiment, the safety latch (2246, 2248, 2249) comprises three components, a pin (2246), a ball (2248) and a notch (2249). The safety latch (2246, 2248, 2249) is positioned at the side of the cover tube (1400). As shown in FIG. 26A, when the pin (2246) is pressed inward so that the ball (2248) is held in the notch (2249), the cover tube (1400) and any components (3260) passing through the cover tube (1400) move as a unit. As shown in FIG. 26B, after the cover tube (1400) and inner components (3260) have moved distally (down) by the required distance B, the pin is pulled outward (arrow). The allows, as shown in FIG. 26C, the inner components (3260) to move distally by a distance A relative to the cover tube (1400), where distance B is larger than distance A.

The longer distal movement (distance B) can be in a range from 0 to 12 mm and the shorter of the two distal movements (distance A) can be in a range from 2 to 6 mm.

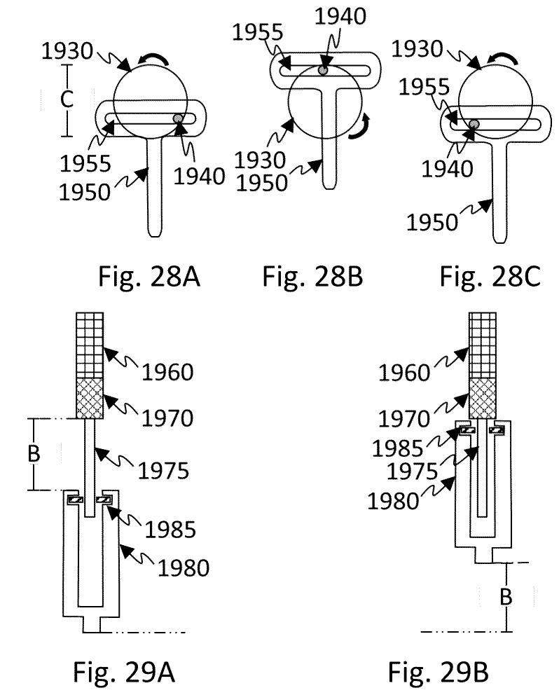

FIGS. 27A-C, 28A-C and 29A-B illustrate embodiments of mechanisms that can convert circular to linear motion, thereby allowing a rotatory motion to control linear movement of the carrier tube and cover tube.

FIG. 27A-C schematically illustrates an embodiment of a device for converting circular motion to linear motion. In this embodiment, the device comprises an inner arm (1910) and an outer arm (1920). The inner arm is fixed at its inner end to a source of rotatory motion. At its outer end, the inner arm (1910) is rotatably attached to a proximal end of the outer arm (1920), the other, distal, end of the outer arm (1920) being constrained (1925) to move linearly, in a proximal-distal direction. The joint between the inner arm (1910) and the outer arm (1920) moves (dashed line) in a circle with a predetermined diameter C. In the exemplary embodiment illustrated, C is 12.5 mm. As the inner arm (1910) rotates from a 9 o'clock position (FIG. 27A) to a 6 o'clock position (FIG. 27B), the distal end of the outer arm (1920) moves the constraint distally. After the inner arm (1910) and the outer arm (1920) have reached the 6 o'clock position, the central spindle of the inner arm (1910) moves, (FIG. 27C), tilting the distal end of the outer arm (1920) and the constraint (1925).

FIG. 28A-C schematically illustrates another embodiment of a device for converting circular motion to linear motion. In this embodiment, the device comprises a wheel (1930) with a stud (1940) near its outer edge and a key (1950) with a slot (1955) in it. The stud (1940) rides in the slot (1955). As the wheel (1930) rotates, carrying the stud (1940) from a 4 o'clock position (FIG. 28A) to a 12 o'clock position (FIG. 28B), the stud (1930) slides in the slot (1955) and carries the key (1950) proximally. Further rotation (FIG. 28C) slides the stud (1940) to the left edge of the slot (1955), driving the key (1940) distally.

FIG. 29A-B schematically illustrates another embodiment of a device for converting circular motion to linear motion. In this embodiment, the device comprises a motor (1960) and one or more gears (1970). Rotation of the motor (1960) rotates the gears, causing the spindle (1975) to rotate. Contact between threads on the spindle (1975) and threads in the tooth (1985) causes the rotation of the spindle (1975) to draw the base (1980) proximally, moving the base (1980) a distance B, from the position seen in FIG. 29A to the position seen in FIG. 29B.

It should be noted that the tool has no sharp angles, which might damage the retina.

The tool can be constructed of a plurality of parts, or it can be constructed from a single tube.

The fluid inlet at the proximal end of the tool and the open end of the carrier tube at the distal end of the tool allows fluid flow through the tool.

The implant, although resting on the implant carrier and holdable by the implant carrier, does not adhere to the implant carrier.

Since the tool can be sealed at both ends before use and implants are preferably inserted into the tool before shipping, at a designated facility, the tool can need no further preparation at the operation site before use since the implant(s) are shipped within it.

The implant can be held to the carrier, for non-limiting example, via a locking mechanism, by capillary forces, by a vacuum, by fluid flow, by fluid pressure and any combination thereof.

In some embodiments, in order to allow a minimally invasive procedure, the carrier and implant are folded into a smaller shape. The implant and/or carrier can be folded or rolled into a tube shape. In some variants of these embodiments, the carrier plus implant is placed within a protecting cannula or other holder. In other variants of these embodiments, the carrier itself forms a cannula shape.

In embodiments where the implant is folded or rolled, it is preferably unfolded or unrolled at the time of emplacement, inside the eye or other body part where it is to be implanted.

Preferably, only a single side of the implant (the non-cell side or underside) contacts the carrier or any part of the tool, thereby protecting the cells in the implant.

Preferably, the implant is removed from the carrier and urged to the implantation site without mechanical contact between the implant and the tool. Preferably, the means of removal and urging, is fluid flow, gentle vibration or both, or by placing the implant on the tissue and using the tissue-implant forces.

In some embodiments, the tool comprises a mechanism for providing feedback of the carrier location. This feedback can be visual (for non-limiting example, an endoscope or a camera or colored portions of the proximal end of the carrier), mechanical (for non-limiting example, one or more detents enabling a user to feel an amount of extension or an angle) or electrical (e.g., impedance change, voltage change).

In some embodiments, the tool comprises a mechanism for providing feedback if the implant/graft is on the carrier. This feedback can be visual (for non-limiting example, an endoscope or a camera or colored portions of the proximal end of the carrier), mechanical or electrical (impedance change sensed from the carrier, voltage change sensed from the carrier).

In some embodiments, at least a portion of the tool has a coating. A coating can by hydrophilic or hydrophobic; and different portions of the tool can have different coatings. A coating can help keep an implant in place, can assist with release of the implant, and any combination thereof.

The tool can comprise a cap, a vial, a flask, a container and any combination thereof. The tool can contain fluids such as, but not limited to, cell medium, other components to ensure viability of implant cells and any combination thereof. In some variants of these embodiments, at least one of the cap, vial, flask and container also comprises an air inlet, a membrane or both to keep cells viable. In some variants of these embodiments the fluids are constantly circulated through or around the tool, replaced regularly every predefined period of time, and any combination thereof.

In some embodiments, the temperature of a tool with a loaded implant is controlled during shipping and storage, with the temperature kept in a range between 4° C. and 25° C., to maintain implant viability. In other embodiments, the temperature of a tool with a loaded implant is controlled during shipping and storage, with the temperature kept in a range between 30° C. and 38° C.

In preferred embodiments, the tool is connectable to operating room facilities such as, but not limited to, a source of fluid, a source of vacuum, a positioning robot, a light source, power and any combination thereof.

The implant can also comprise a source of slow-release medication, a source of slow-release medication can be emplaced with the implant, and any combination thereof.

The actuation of ejection of the implant carrier from the tool can be effectuated by a mechanism comprising a member of a group consisting of a slider, a pin, a converter from circular motion to linear motion, a motor and gear, a motor and cam, a wheel, a knob, a screw, and any combination thereof.

The actuation of detachment of the implant from the carrier can be effectuated by a mechanism comprising a member of a group consisting of fluid flow, mechanical vibration, an ultrasonic actuator, gravitational force, adhesion force between the implant and the retina and any combination thereof.

Preferably, the tool comprises a protective sealing mechanism at a distal end of the tool. Typically, the seal will be broken or the cap removed at the start of an implantation procedure. The seal can be opened, pierced, removed and any combination thereof.

The protective sealing mechanism can be a cap, a diaphragm, any other conventional means of protecting a delicate inner part and of preserving sterility of an inner part, and any combination thereof. The protective sealing mechanism can allow at least one of a group consisting of diffusion of gas therethrough and a nutrient path therethrough.

Fluid flow can be used to detach the implant from the carrier, position the implant, transfer liquid, maintain viability of the implant, maintain subretinal bleb shape and size, maintain intraocular pressure, and any combination thereof.

Preferably, the implant is maintained in a temperature range between 4° and 38° C. between manufacture and use.

Preferably, the implant is maintained in solution between manufacture and use.

In some embodiments, at least a portion of the tool is colored to improve visibility of a member of a group consisting of an implant, a retina, a bleb, at least a portion of a component of the tool and any combination thereof. The color can be darker than another component, lighter than another component, fluorescent, green, and any combination thereof.

The tool can be controlled automatically, semi-automatically, and manually. Automatic and/or semi-automatic control can be via a robot. In some embodiments, vibration damping is provided.

In some embodiments, the tool comprises a lighting mechanism configured to provide light at the tool's distal end, enabling a user to more clearly see the implantation area and, therefore, to more accurately position the implant in the eye or other body part. The lighting mechanism can comprise an LED, a fiber optic cable, and any combination thereof. The tool can comprise a camera or other visual sensor configured to display a view of a region near the distal portion of said insertion tool.

In some embodiments, the tool comprises a mechanism for rotating the implant while maintaining the direction of implantation, to ensure proper placement and alignment of the implant.

In some embodiments, there is a slow and constant flow of fluid (0.05-1 ml/min) through the tool to maintain the bleb shape and assist in detaching the implant.

As noted above, according to another embodiment, the carrier (1290) is substantially curved when the same is outside the carrier tube (1260) and not flat, such that when the implant (graft) is placed on the same, it will assume its curvature (even before entrance into the carrier tube (1260). Obviously, when inside the carrier tube (1260), the carrier (1290) and the implant will assume a rolled configuration, as detailed above.

According to some embodiment, the radius of curvature of the carrier (1290) is in the range of 0.25-10 mm; preferably, in the range of 1.0-3 mm.

According to another embodiment, the carrier tube (1260) is characterized by radius in the range of 0.1-4 mm; preferably, in the range of 0.1-0.7 mm.

Such curvature of the carrier (1290) and the carrier tube (1260) is important to facilitate insertion of the carrier (1290) and the substrate/implant/graft (1000) into the carrier tube (1260) and thus, taking a shape selected from a group consisting of folded, rolled and bent.

In some embodiments, following the insertion and opening of the tool in the sub-retina space (the bleb), the flow of fluid is used to extract the implant (the graft) out of the tool and into its place in the sub retina.

During said extraction it is essential that the graft maintains its orientation (so as to allow the correct positioning of the graft on the retina, without damaging the sensitive cells or collagen layers).

Such extraction can be performed by one or more of the following:
1. Flowing liquid through the carrier tube (1260) to push the implant (1000) forward and out of the tool;
2. Flowing liquid through the carrier tube (1260) to lift (and/or push) the graft (1000);
3. Tilting the tool to a certain angle that will allow gravity forces to pull the graft (1000) out of the tool;
4. Vibrating the tool by manual or automatic manner—tapping, electric motor, piezoelectric transducer, ultrasound means (and any combination thereof) to pull the graft (1000) out of the tool;
5. Reciprocal movement of the carrier (1290) in and out of the carrier tube (1260) to enable the release of the graft (1000).

According to an embodiment where liquid is used to facilitate extraction of the implant by lifting or pushing the same out of the tool, an elevated mechanical element is provided in the carrier tube that will keep one side of the graft (1000) in a certain elevated distance from the graft carrier (1290), allowing the liquid to flow below the graft.

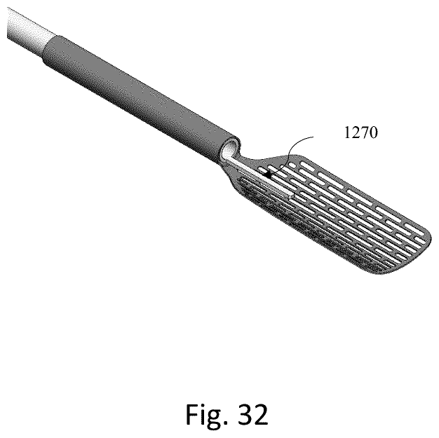

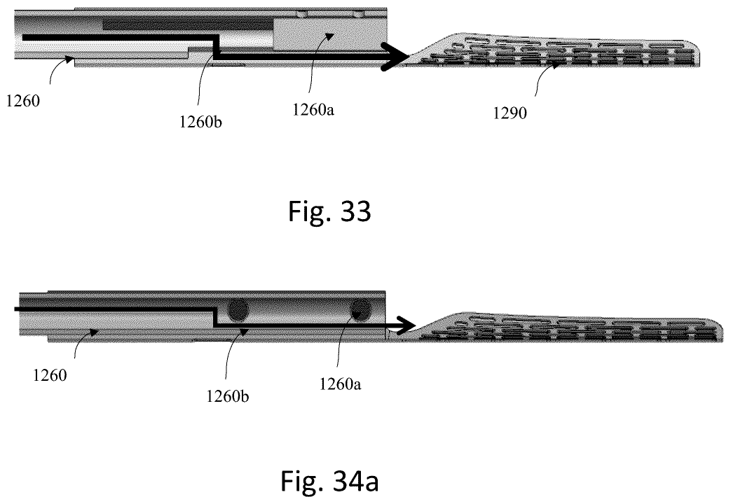

FIG. 32 illustrates such an embodiment. In this figure, an extension 1270 throughout which the flow of fluids is used for elevating implant 1000 and facilitating its extraction out of the carrier. According to another embodiment, means for directing the fluid flow in the desired direction is provided either inside the carrier tube or on its opening end. Reference is now made to FIGS. 33-36.

As seen in FIG. 33, the carrier tube (1260) comprising at least one element 1260a (shown as a rod in FIG. 33) essentially blocking substantial portion of the inner diameter of the carrier tube (1260); thus, forcing the fluid flow (illustrated as arrow 1260b) to flow to carrier 1290 from the bottom portion of the carrier tube (1260).

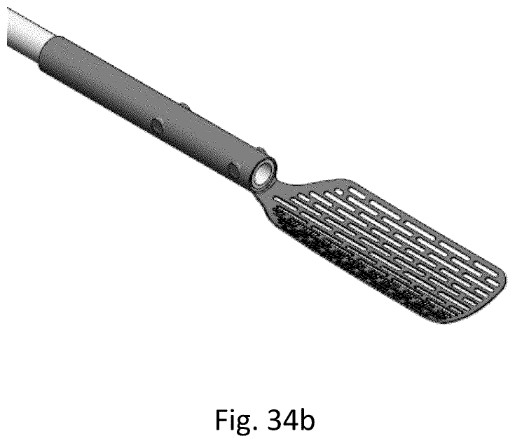

FIGS. 34a-34b illustrate another fluid flow directing means. In this figure, element 1260a is embodied as two rods disposed within carrier tube 1260. Again, as in FIG. 33, the fluid flow (illustrated as arrow 1260b) is forced to flow to carrier 1290 from the bottom portion of the carrier tube (1260).

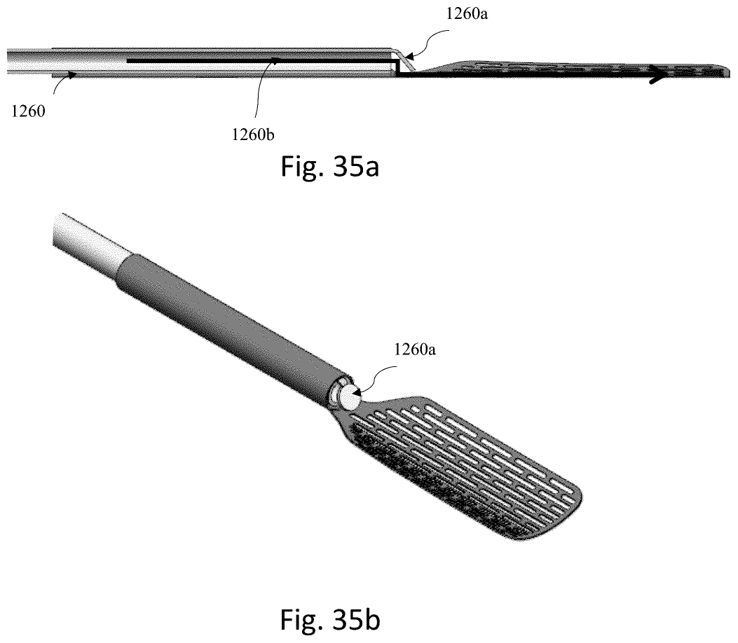

FIGS. 35a-35b illustrate another fluid flow directing means. In this figure, the distal-most part 1260a of the carrier tube 1260 is bent (with or without means to control the bending angle. Again, as in FIGS. 33 and 34, the fluid flow (illustrated as arrow 1260b) is forced to flow to carrier 1290 from the bottom portion of the carrier tube (1260).

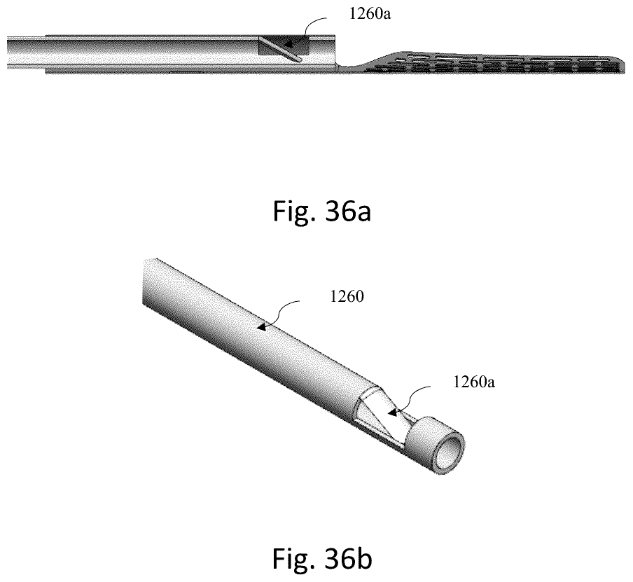

FIGS. 36a-36b illustrate another fluid flow directing means. In this figure, a distal part 1260a of the carrier tube 1260 is cut and bent (with or without means to control the bending angle. Again, as in FIGS. 33-35, the fluid flow (illustrated as arrow 1260b) is forced to flow to carrier 1290 from the bottom portion of the carrier tube (1260).

According to another embodiment, means for increasing the flow velocity are provided.

Such means could be e.g., narrowing the carrier tube, enlarging the tube, blocking part thereof, adding an elevated or protruding part inside or at the opening of the tube, or any combination thereof.

According to one embodiment the fluid flow rate can be controlled automatically or manually.

According to one embodiment, the carrier tube could be connected to a syringe, or a motorized device such as the constellation machine, a syringe pump, a pressure-controlled pump, or any combination thereof.

Since the dimensions of the graft (1000), the carrier (1290) and the carrier tube (1260) are in the millimetric scale, and the volumetric flow values are relatively small (tens to hundreds of microliters per seconds), it can be understood that the Reynolds numbers of the flow are relatively small. Therefore, the flow lines of the fluids that are ejected from the tool in the sub-retina tend to spread after a few millimeters from the carrier tube opening, and might not be effectively used to push or lift the graft.

Thus, to support the required directional flow, according to one embodiment of the present invention, the opening of the carrier tube (1260) is narrowed resulting in an increase of the flow velocity and effectively the Reynolds numbers.

Additionally, as described above, adding a mechanical element that will direct the flow toward or below the graft, can affect the flow lines and enhance the pushing/lifting effects.

Although in some cases it is necessary to lift the graft from the carrier, in some other cases pushing the ultra-thin graft with flow of fluid might result in folding of the graft, or flipping it with the cells side facing toward the Bruch's membrane. Therefore, in these cases it is preferred to direct the flow of liquid parallel to the graft, using the viscosity of the liquid and the adhesion forces between the liquid and the graft to push the graft out of the tool.

In such cases it would be preferred to keep the graft (1000) in proximity to the graft carrier (1290) while pushing it forward to avoid flow lines getting below the graft and flipping it.

Reference is now made to FIG. 30 illustrating a small protruding element (e.g., pin 1500) at the end of the carrier tube (1260) as a mechanical element to direct the flow (illustrated by arrows 1501) upwards and increase flow velocity (further embodiment are illustrated in FIGS. 33-36).

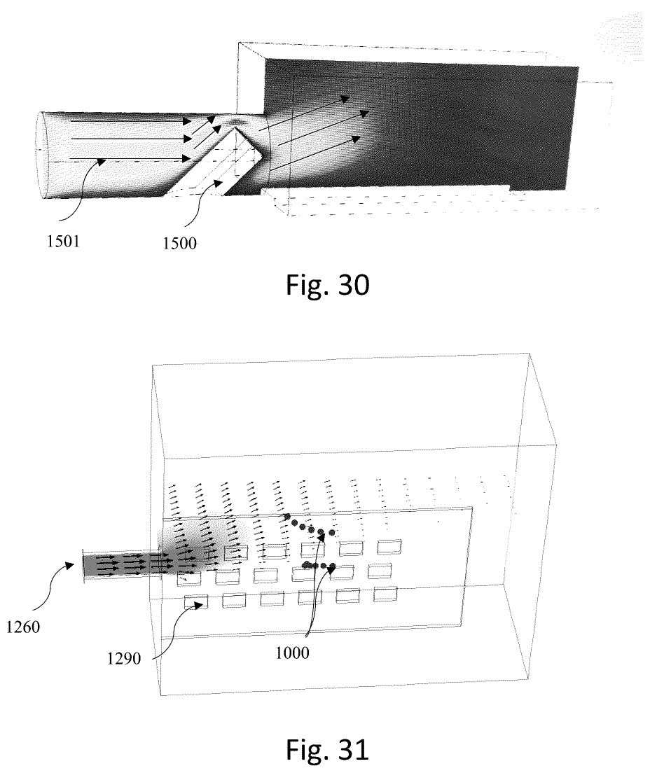

Reference is now made to FIG. 31 illustrating a narrowed carrier tube (1260) opening to increase flow velocity. What is seen in the figure is the cross-sectional view of the flow velocity map after fluid injection (illustrated as black arrows), where the dark spheres on the bottom represent the graft (1000) positioning on the carrier (1290) at different times following fluid injection.

According to another embodiment of the present invention, the flow of fluid does not, merely, facilitate the extraction of the implant (1000) but also it is utilized as means for ensuring any air bubbles, trapped in the tool, are extracted out.

According to another embodiment, the tool would be comprised of two essential elements. A proximal part and a distal part reversibly connected to each other.

In this embodiment, the distal part 3701 (including the carrier (1290), the implant (1000) and at least a portion of the carrier tube (1260)) could be cryopreserved and only upon implantation it would be taken out of the cryopreserved (and thawed). Then, it can be sealingly connected to the proximal part of the tool.

Thus, the distal part 3701 (including the carrier (1290), the implant (1000) and at least a portion of the carrier tube (1260)) is maintained in cryopreservation until implantation is required. Upon the need for implementation, the distal part 3701 is thawed and then coupled to the proximal part to provide the two-parts implementation tool ready for implantation.

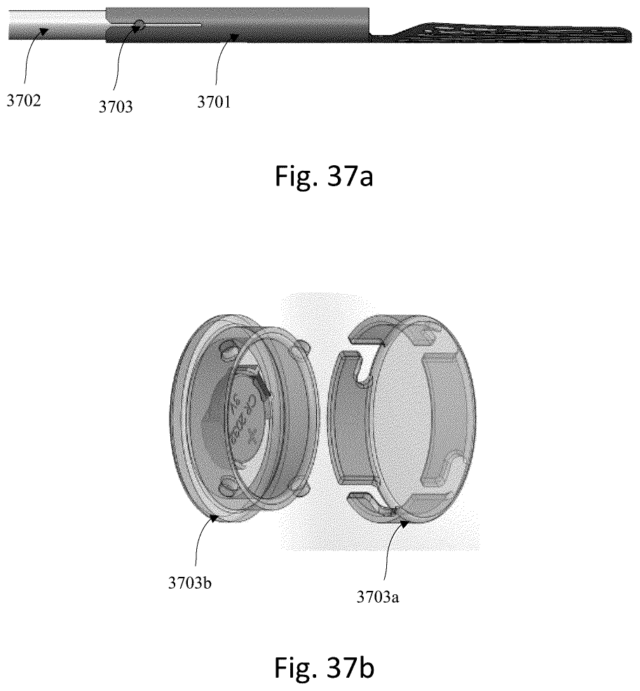

Reference is now made to FIGS. 37a-37b illustrating such an embodiment.

As seen in FIG. 37a, the tool comprising a distal part 3701 and a proximal part 3702, reversibly coupled by coupling means 3703.

It is appreciated that coupling means 2703 could be selected from a group consisting of mechanical means, magnetic means and any combination thereof.

Reference is now made to FIG. 37b, illustrating one example of clip-on mechanical-based coupling means 3703. As seen in the figure, one part of coupling means 3703a is the proximal end side of the distal part 3701 of the tool. Also seen in the figure is the second part of coupling means 3703a which is the distal end side of the proximal part 3702 of the tool.

When needed, the distal part (containing the implant 1000) 3701 is extracted from cryopreserved, and connected to the proximal part 3702; resulting in the tool being fully operative and ready for implantation.

Thus, according to another embodiment of the present invention, an insertion tool for inserting an implant is provided. According to this embodiment, the insertion tool is characterized by a distal part and a proximal part reversibly interconnected to each other.

Such configuration enables the cryopreservation of only the distal part (and, thus, storage of the same). Only upon implantation, the distal end (along with the implant) is thawed.

After such thawing, the distal part is sealingly connected with the proximal part to result in a functioning ready-to-use implantation tool.

Thus, in other words, the distal end is cryopreserved and stored; and, when implantation is required, the distal part is removed from cryopreservation (and thawed) and is ready for use by coupling the same to the proximal part).

The distal part comprises (a) an implant carrier configured to hold an implant; (b) a body having at least two configurations, a first body configuration with said implant carrier at least partially contained within said body and said implant held by said implant carrier and a second body configuration with said implant carrier outside said body.

The proximal part comprises a proximal part body, adapted to be sealingly reversibly connected to said body in said distal part.

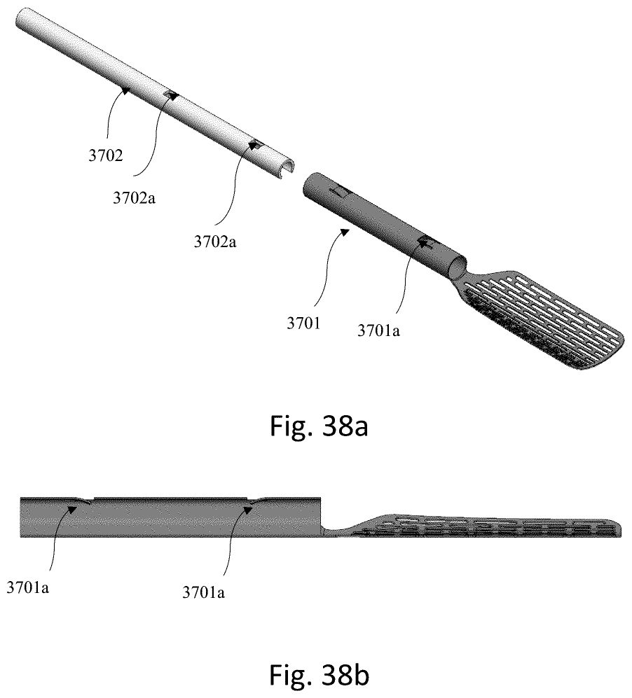

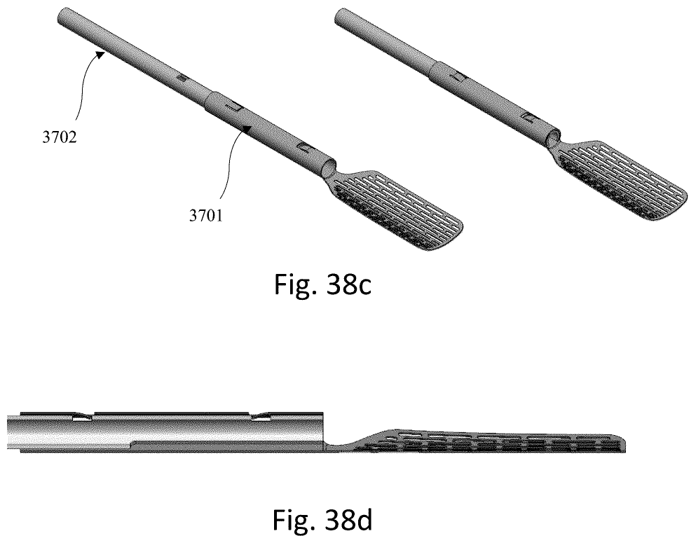

According to one embodiment of the present invention, the cryopreservation solution is selected from a group consisting of CryoStor® cell cryopreservation media, CS0, CS2, CS5, CS10. Additionally or alternatively, the cryopreservation can be done in HumanSerum/DMO, Cell media/DMSO solution, and other cryopreservation solutions—STEM-CELLBANKER®, HSC-BANKER®, NutriFreez® D10 Cryopreservation Medium and any combination thereof. Reference is now made to FIGS. 38a-38d illustrating another embodiment of the reversible coupling of the proximal part 3702 and a distal part 3701.

According to this embodiment, the proximal part 3702 comprises at least one cut (or aperture) 3702a on the outer peripheral circumference thereof.

As seen in FIG. 38a, the carrier tube (1260) of the distal part 3701 comprises at least one tab 3701a. Tab 3701a are pre-shaped as an internally protruding element (bulging into the inside of the carrier tube (1260) of the distal part 3701).

Reference is now made to FIG. 38b illustrating a cross sectional view of the carrier tube (1260) of the distal part 3701 after the treatment to said at least one tab 3701a. As illustrated in the FIG. tab 3701a internally protrude into the inside of the carrier tube (1260) of the distal part 3701.

Reference is now made to FIG. 38c, illustrating the coupling (assembly) of the distal part 3701 and the proximal part 3702 (e.g., prior to use and implantation of the graft). In this case the carrier tube (1260) of the proximal part 3702 has a smaller diameter than the carrier tube (1260) of the distal part 3701 and hence sliding the proximal part within the distal part will couple the two parts together.

Reference is now made to FIG. 38d illustrating the full assembly of the distal part 3701 and the proximal part 3702.

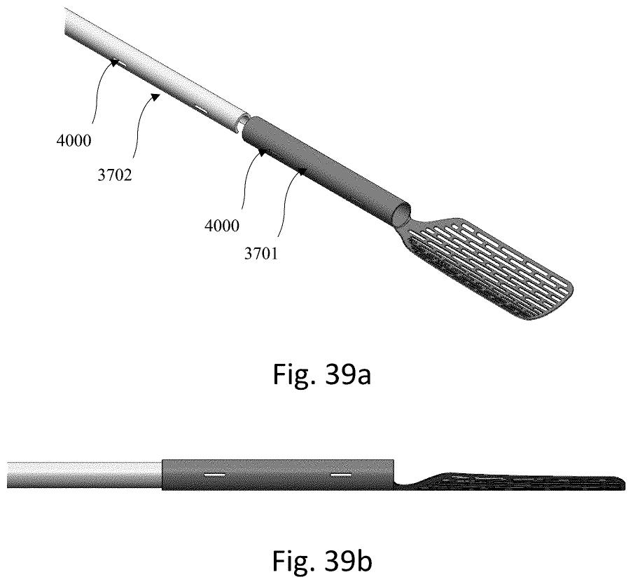

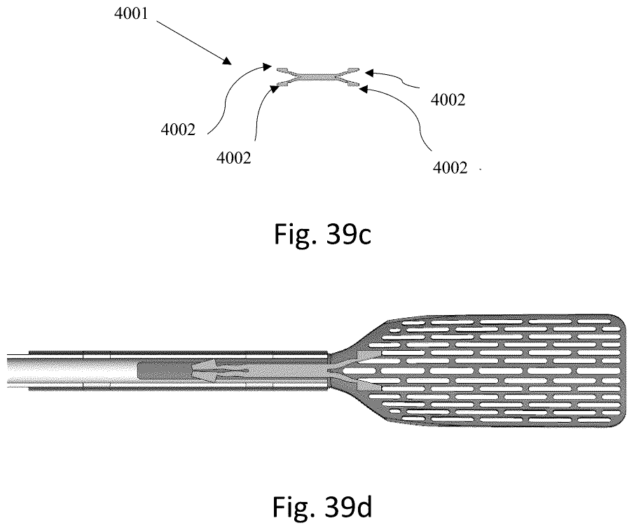

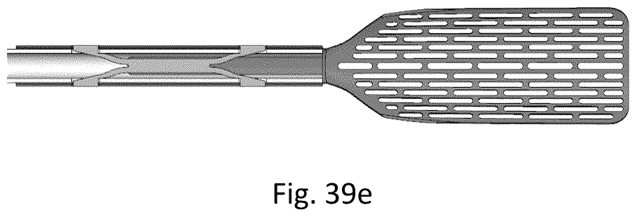

Reference is now made to FIGS. 39a-39e another embodiment of the present invention. According to this embodiment, the carrier tube (1260) of the distal part 3701 and the carrier tube (1260) of the distal part 3702 comprises at least one aperture 4000.

When use of the implant is needed (i.e., implantation of the graft), the carrier tube (1260) of the distal part 3701 and the carrier tube (1260) of the distal part 3702 will be assembled together such that the aperture 4000 of the carrier tube (1260) of the distal part 3702 is aligned with the aperture 4000 of the carrier tube (1260) of the proximal part 3701 (see FIG. 39b). As noted above, the carrier tube (1260) of the proximal part 3702 has a smaller diameter than the carrier tube (1260) of the distal part 3701 and hence sliding the proximal part within the distal part will assemble the two parts together (see FIG. 39b).

Securing the two parts together will be enabled by means of a clip 4001.

Reference is now made to FIG. 39c illustrating such a clip 4001. As illustrated in the figure, the clip is characterized by at least two elastic arms 4002. Said arms having spring-like properties. According to one embodiment, the clip 4001 is characterized by at least 2 configurations: a first configuration, in which no external force is applied on the arms 4002; and, the same are spaced apart from each other; and, a second configuration, in which external force is applied on the arms 4002; and, the same are closed to one another.

In FIG. 39c the first configuration is illustrated. The second configuration is illustrated in FIGS. 39d-39e.

Reference is now made to FIGS. 39d-39e illustrating the securing step of the proximal part 3702 and the distal part 3701 by means of the clip 40001.

As seen in FIG. 39d, once the proximal part 3702 and the distal part 3701 are assembled and the apertures 4000 are aligned, clip 4001 is inserted into the carrier tube (1260) of the proximal part 3702.

Insertion of the clip 4001 into the carrier tube (1260) of the proximal part 3702 converts the clip 4001 from the first configuration to the second configuration and arm 4002 are moved closer together (it should be noted that should the clip be extracted out of the carrier tube (1260) of the proximal part 3702, arm 4002 will be spaced apart again).

Reference is now made to FIG. 39e illustrating the full insertion of the clip into the carrier tube (1260) of the proximal part 3702. Once fully inserted, arms 4002 will align with apertures 4000; thereby securing the carrier tube (1260) of the proximal part 3702 within carrier tube (1260) of the distal part 3701.

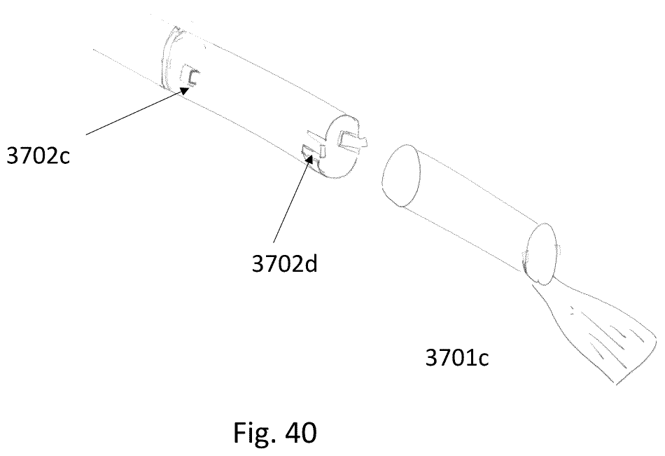

Reference is now made to FIG. 40 illustrating another mechanical means for coupling the proximal part 3702 and the distal part 3701 together. According to this embodiment, the carrier tube (1260) of the proximal part 3702 will comprise at least one depth imitator 3702c on the one side and at least one snap (or any other mechanical coupler) 3702d on the other side.

The carrier tube (1260) of the distal part 3701 will comprise at least one notch 3701c. Such notch 3701c will prevent the carrier 1290 from any rotational movement, once the carrier tube (1260) of the distal part 3701 is inserted and positioned within the carrier tube (1260) of the proximal part 3702. Once fully inserted, snap 3702d will be coupled to said at least one notch 3701c; thereby preventing an unwanted movement.

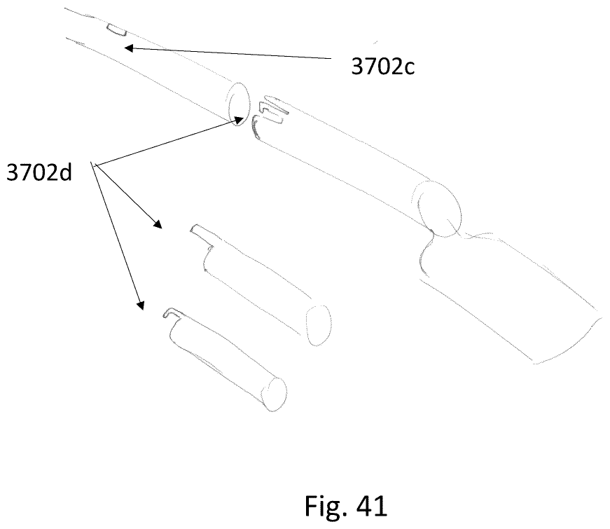

Reference is now made to FIG. 41 illustrating several examples of a snaps 3702d positioned on the carrier tube (1260) of the distal part 3701. According to this embodiment, the carrier tube (1260) of the proximal part 3702 will comprise at least one notch 3702c to be coupled with said at least one snap 3702d.

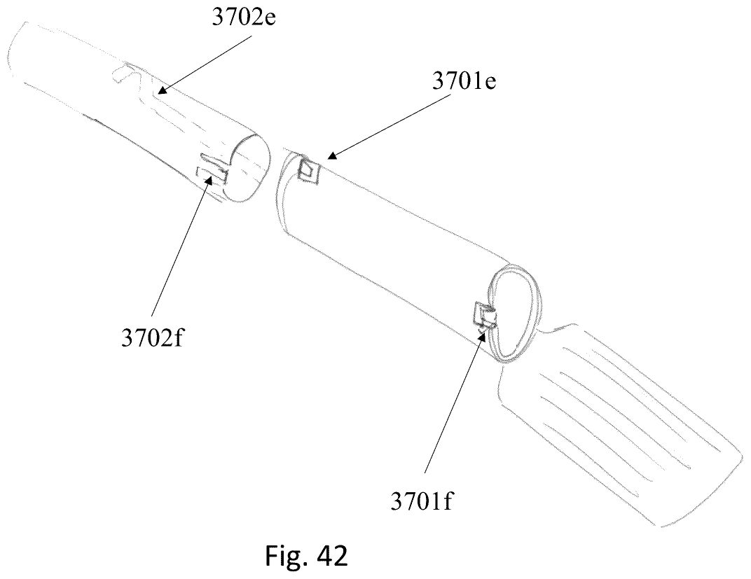

Reference is now made to FIG. 42 illustrating another mechanical means for coupling the proximal part 3702 and the distal part 3701 together.

According to this embodiment, the carrier tube (1260) of the distal part 3701 comprises at least one tab (or protrusion) 3701e and at least one notch 3701f (for full and secure coupling with the carrier tube (1260) of the proximal part 3702).

According to this embodiment, the carrier tube (1260) of the proximal part 3702 comprises at least one slot 3702e for said tab 3701e to be inserted thereto; and at least one snap 3702f, adapted for coupling with said at least one notch 3701f.

It should be noted that according to another embodiment of the present invention, in case where cryopreservation is used for the distal part 3701 an additional element will be used (e.g., a tube) to hold the carrier 1290 closed while it is cryopreserved. Thus, the carrier 1290 and the implant are kept closed.

It should be noted that as disclosed above, in the 2 parts tool the carrier (1290) is curved (and not straight) when is outside the carrier tube 1260 of the distal part 3701.

According to another embodiment of the present invention, in order to ensure the graft/implant 1000 is not removed from the carrier 1290 during the cryopreservation.

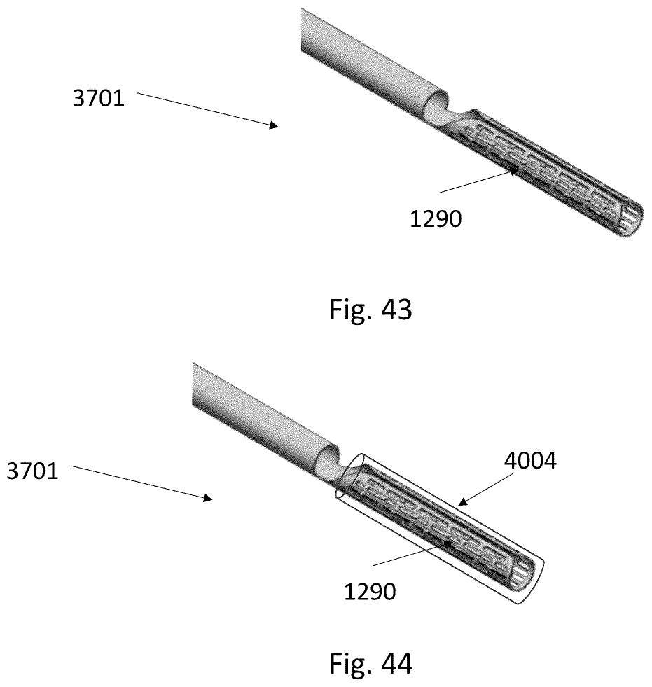

Thus, according to one embodiment of the present invention, the carrier 1290 (with the implant 1000 placed thereupon) is substantially rolled outside the carrier tube 1260 of the distal part 3701 (see FIG. 43, where only the distal part 3701 is illustrated).

As seen in the FIG. 43, the carrier 1290 (and the implant 1000 thereupon) is substantially rolled (and not just curved)—i.e., the carrier 1290 is in its first carrier configuration (namely, folded, rolled and bent). Such configuration ensures the implant 1000 is not removed from the carrier during the cryopreservation.

Reference is now made to FIG. 44 illustrating distal part 3701 and the carrier 1290 (and the implant 1000 thereupon) is in its first carrier configuration (namely, folded, rolled and bent). According to another embodiment of the present invention, an additional element (e.g., a closed tube 4004) in reversible communication with carrier 1290, adapted to ensure said carrier 1290 is maintained in said first configuration (namely, folded, rolled and bent) when outside the tube carrier 1260. In other words, such element is adapted to restrict the movement of said carrier 1290 from said first configuration (namely, folded, rolled and bent) to the second configuration (i.e., a curved shaped, as disclosed hereinabove). In this case, tube 4004 prevents carrier 1290 from opening and possibly enabling the removal of the implant 1000 from the carrier 1290 during cryopreservation. Thus, element 4004 maintains carrier 1290 folded\rolled to maintain the implant 1000 thereupon during cryopreservation and prevent the possibility that the implant would mistakenly be removed from the carrier during the cryopreservation.

Once implantation is required and the distal end is thawed, element 4004 can be removed (and thus, the restriction on carrier 1290 is removed) to enable carrier 1290 to be slightly curved when outside carrier tube 1260 (to facilitate implantation of the implant in the patient's eye).

It should be noted that such movement restrictor element 4004 could be used in combination with any of the embodiments disclosed above relating to the two parts tool and the ability to cryopreserve only the distal part of the tool (such that only prior to implantation the distal part is connected to the proximal part to facilitate the implantation).

It should further be noted that restrictor element 4004 could be any mechanical\electrical\magnetic means known in the art to restrict movement (i.e., reconfiguration from the first configuration to the second configuration) of carrier 1290.

Thus, it is one object of the present invention to provide, a two parts insertion tool for inserting an implant. According to this embodiment, the insertion tool is characterized by a distal part and a proximal part reversibly and sealingly interconnected to each other.

The distal part comprises (a) an implant carrier configured to hold an implant; (b) a body having at least two configurations, a first body configuration with said implant carrier at least partially contained within said body and said implant held by said implant carrier and a second body configuration with said implant carrier outside said body.

The proximal part comprises a proximal part body, adapted to be sealingly reversibly connected to said body in said distal part;
wherein, when implantation is required, said distal part is sealingly coupled to said proximal part.

It is another object of the present invention to disclose the two parts insertion tool as defined above, wherein only the distal part is cryopreserved.

It is another object of the present invention to disclose the two parts insertion tool as defined above, when implantation is required, the distal part is thawed.

It is another object of the present invention to disclose the two parts insertion tool as defined above, when implantation is required, the distal part is reversibly and sealinly coupled to the proximal part.

It is another object of the present invention to disclose the two parts insertion tool as defined above, for inserting an implant, where said implant having an implant lower side and an implant upper side, wherein said implant carrier is configured to maintain a position of the implant and a shape of the implant and functionality of said cells during such time as the implant carrier is contained by said body, and during an implantation procedure;

It is another object of the present invention to disclose the two parts insertion tool as defined above, wherein said implant carrier has at least two configurations, a first carrier configuration where the implant carrier has a shape selected from a group consisting of folded, rolled and bent, and a second carrier configuration where the implant carrier has a curved shape.

It is another object of the present invention to disclose the two parts insertion tool as defined above, wherein said implant carrier is in said first carrier configuration at such times as said implant carrier is within said body and said implant carrier is transformable to said second carrier configuration during such times as said implant carrier is outside of said body.

It is another object of the present invention to disclose the two parts insertion tool as defined above, wherein said implant carrier is retractable into said body.

It is another object of the present invention to disclose the two parts insertion tool as defined above, wherein said implant carrier is movable outward from said body.

It is another object of the present invention to disclose the two parts insertion tool as defined above, wherein said body is openable.

It is another object of the present invention to disclose the two parts insertion tool as defined above, wherein said implant carrier is a part of said body.

It is another object of the present invention to disclose the two parts insertion tool as defined above, wherein said implant carrier comprises a member of a group consisting of Nitinol, Polyimide (Kapton), stainless steel and any combination thereof.

It is another object of the present invention to disclose the two parts insertion tool as defined above, wherein said implant carrier comprises a coating selected from a group consisting of a hydrophilic coating, a hydrophobic coating and any combination thereof.

It is another object of the present invention to disclose the two parts insertion tool as defined above, wherein said implant is made of a material selected from a group consisting of soft, foldable, bendable and any combination thereof.

It is another object of the present invention to disclose the two parts insertion tool as defined above, additionally comprising an implant removal mechanism selected from a group consisting of an arm contactable to said implant, a wire attachable to said implant, fluid flow, vibration, and any combination thereof.

It is another object of the present invention to disclose the two parts insertion tool as defined above, wherein said fluid flow is adapted to perform at least one selected from a group consisting of lifting said implant from said implant carrier, pushing said implant out of said tool, remove all air bubbles in said tool, and any combination thereof.

It is another object of the present invention to disclose the two parts insertion tool as defined above, additionally comprising at least one element, adapted to direct said fluid flow to a predetermined direction.

It is another object of the present invention to disclose the two parts insertion tool as defined above, additionally comprising at least one element, adapted to elevate said implant from said graft carrier, allowing said fluid to flow below said implant.

It is another object of the present invention to disclose the two parts insertion tool as defined above, wherein said vibration is provided by at least one selected from a group consisting of tapping, electric motor, piezoelectric transducer, ultrasound means and any combination thereof.

It is another object of the present invention to disclose the two parts insertion tool as defined above, additionally comprising means adapted to increase said fluid flow velocity.

It is another object of the present invention to disclose the two parts insertion tool as defined above, wherein said increase in said fluid flow velocity is provided by means selected from a group consisting of narrowing said body, enlarging said body, blocking part thereof, adding an elevated or protruding part inside or at the opening of said body, or any combination thereof.

It is another object of the present invention to disclose the two parts insertion tool as defined above, wherein said fluid flow is controlled either manually or automatically.

It is another object of the present invention to disclose the two parts insertion tool as defined above, wherein said implant carrier is configured to position said implant into a member of a group consisting of a subretinal bleb and an anterior chamber.

It is another object of the present invention to disclose the two parts insertion tool as defined above, wherein said implant is configured for ophthalmic applications.

It is another object of the present invention to disclose the two parts insertion tool as defined above, wherein said implant is a retinal implant.

It is another object of the present invention to disclose the two parts insertion tool as defined above, wherein said implant is a corneal implant.

It is another object of the present invention to disclose the two parts insertion tool as defined above, wherein said implant comprises a member of a group consisting of retinal pigment epithelium cells, photo-receptor cells and any combination thereof.

It is another object of the present invention to disclose the two parts insertion tool as defined above, wherein said implant comprises a member of a group consisting of collagen, hydrogel, a medication and any combination thereof.

It is another object of the present invention to disclose the two parts insertion tool as defined above, wherein said medication is a slow-release medication.

It is another object of the present invention to disclose the two parts insertion tool as defined above, wherein said implant diameter is in a range of 0.5 mm to 8 mm.

It is another object of the present invention to disclose the two parts insertion tool as defined above, wherein said implant thickness is in a range of 2 μm to 500 μm.

It is another object of the present invention to disclose the two parts insertion tool as defined above, wherein said implant length is in a range of 0.5 mm to 8 mm.

It is another object of the present invention to disclose the two parts insertion tool as defined above, wherein said implant width is in a range of 0.5 mm to 8 mm.

It is another object of the present invention to disclose the two parts insertion tool as defined above, wherein the trocar diameter is no larger than 20 G (0.9 mm).

It is another object of the present invention to disclose the two parts insertion tool as defined above, wherein the trocar diameter is larger than 20 G (0.9 mm).

It is another object of the present invention to disclose the two parts insertion tool as defined above, wherein actuation of removal of said implant from said implant carrier is effectuated by a mechanism comprising a member of a group consisting of a slider, a pin, a converter from circular motion to linear motion, a motor and gear, a motor and cam, a wheel, a knob, a screw, and any combination thereof.

It is another object of the present invention to disclose the two parts insertion tool as defined above, additionally comprising a protective seal at a distal end of said insertion tool.

It is another object of the present invention to disclose the two parts insertion tool as defined above, wherein said protective seal is selected from a group consisting of a cap, a diaphragm and any combination thereof.

It is another object of the present invention to disclose the two parts insertion tool as defined above, wherein said protective seal allows at least one of a group consisting of diffusion of gas therethrough and a nutrient path therethrough.

It is another object of the present invention to disclose the two parts insertion tool as defined above, wherein said insertion tool is configured to enable fluid flow to perform a member of a group consisting of detaching said implant from said implant carrier, positioning said implant, transferring liquid, maintaining viability of said implant, maintaining intraocular pressure, maintaining retinal bleb size, and any combination thereof.

It is another object of the present invention to disclose the two parts insertion tool as defined above, wherein said implant is emplaceable within said insertion tool prior to shipment and said implant is maintainable in a usable condition between said emplacing and use.

It is another object of the present invention to disclose the two parts insertion tool as defined above, wherein said implant is maintainable in a temperature range of 4° C. to 25° C. between said emplacing and said use.

It is another object of the present invention to disclose the two parts insertion tool as defined above, wherein said implant is maintainable in a temperature range of 30° C. to 38° C. between said emplacing within said insertion tool and said use.

It is another object of the present invention to disclose the two parts insertion tool as defined above, wherein said implant is maintainable in solution between said emplacing within said insertion tool and said use.

It is another object of the present invention to disclose the two parts insertion tool as defined above, wherein said fluid comprises a member of a group consisting of balanced saline solution (BSS), BSS Plus, BSS Plus with additional viscous content such as Hyaluronic acid, and a CO₂ independent medium.

It is another object of the present invention to disclose the two parts insertion tool as defined above, wherein a shape of a portion of said body is selected from a group consisting of bent, controllably bendable, straight and any combination thereof.

It is another object of the present invention to disclose the two parts insertion tool as defined above, wherein said bent or controllably bendable portion is passable through at least one of a trocar and a cannula.

It is another object of the present invention to disclose the two parts insertion tool as defined above, wherein, in said bent configuration, an angle between a longitudinal axis of said body and a longitudinal axis of said bent portion is in a range from 0° to 90°.

It is another object of the present invention to disclose the two parts insertion tool as defined above, wherein at least a portion of a member of a group consisting of said body, said cap, said implant carrier and any combination thereof is transparent.

It is another object of the present invention to disclose the two parts insertion tool as defined above, wherein said transparency enables visibility of at least a portion of a member of a group consisting of said implant, said implant carrier and any combination thereof.

It is another object of the present invention to disclose the two parts insertion tool as defined above, wherein at least a portion of said insertion tool is colored to improve visibility of a member of a group consisting of said implant, at least a portion of a component of said insertion tool and any combination thereof.

It is another object of the present invention to disclose the two parts insertion tool as defined above, wherein said color is selected from a group consisting of darker than another component, lighter than another component, fluorescent, green, and any combination thereof.

It is another object of the present invention to disclose the two parts insertion tool as defined above, wherein control of said insertion tool is selected from a group consisting of robotic, automatic, semi-automatic, manual, vibration damping and any combination thereof.

It is another object of the present invention to disclose the two parts insertion tool as defined above, additionally comprising a lighting mechanism configured to provide light at a distal end of said insertion tool.

It is another object of the present invention to disclose the two parts insertion tool as defined above, wherein said lighting mechanism comprises a member of a group consisting of an LED, a fiber optic cable, and any combination thereof.

It is another object of the present invention to disclose the two parts insertion tool as defined above, additionally comprising a camera configured to display a view of a region near a distal portion of said insertion tool.

It is another object of the present invention to disclose the two parts insertion tool as defined above, additionally comprising a mechanism for rotating said implant while maintaining a direction of implantation.

It is another object of the present invention to disclose the two parts insertion tool as defined above, wherein said implant is ejectable from said implant carrier.

It is another object of the present invention to disclose the two parts insertion tool as defined above, wherein said implant carrier is in contact with only said implant lower side.

It is another object of the present invention to disclose the two parts insertion tool as defined above, wherein said implant carrier is configured to protect said implant upper side from mechanical stress and shear.

It is another object of the present invention to disclose the two parts insertion tool as defined above, wherein the implant is releasably connectable to the implant carrier, said releasable connection configured, before release, to prevent a member of a group consisting of movement of said implant, folding of said implant and any combination thereof.

It is another object of the present invention to disclose the two parts insertion tool as defined above, additionally comprising a locking mechanism.

It is another object of the present invention to disclose the two parts insertion tool as defined above, wherein said locking mechanism comprises a member of a group consisting of locking tubes comprising holes through which a fluid can be passed, an arm contactable to said implant, a wire attachable to said implant, and any combination thereof,

It is another object of the present invention to disclose the two parts insertion tool as defined above, wherein said insertion tool is configured to allow flow of flow fluid into said insertion tool from a volume near a distal end of said insertion tool.

It is another object of the present invention to disclose the two parts insertion tool as defined above, wherein at least a portion of said insertion tool is configured to enable implant orientation by means of rotation of said at least a portion of said insertion tool.

It is another object of the present invention to disclose the two parts insertion tool as defined above, additionally comprising an insertion stopper, configured to allow adjustment of a distance said implant carrier extends distally from said body of said insertion tool.

It is another object of the present invention to disclose the two parts insertion tool as defined above, wherein said implant carrier is extendable from said body by at least one predetermined distance.

It is another object of the present invention to disclose the insertion tool as defined above, wherein said at least one predetermined distance comprises a first predetermined distance and a second predetermined distance.

It is another object of the present invention to disclose the two parts insertion tool as defined above, wherein said first predetermined distance is in a range from 7 mm to 10 mm and said second predetermined distance is in a range from 2 to 6 mm.

It is another object of the present invention to disclose the two parts insertion tool as defined above, wherein said extending mechanism is selected from a group consisting of a slider, a pin, a converter from circular motion to linear motion, a motor and gear, a motor and cam, and any combination thereof.

It is another object of the present invention to disclose the two parts insertion tool as defined above, wherein said carrier tube comprises a superelastic material.

It is another object of the present invention to disclose the two parts insertion tool as defined above, wherein said carrier tube comprises Nitinol.

It is another object of the present invention to disclose the two parts insertion tool as defined above, wherein said implant carrier comprises a superelastic material.

It is another object of the present invention to disclose the two parts insertion tool as defined above, wherein said implant carrier comprises Nitinol.

It is another object of the present invention to disclose the two parts insertion tool as defined above, wherein said implant carrier comprises holes.

It is another object of the present invention to disclose the two parts insertion tool as defined above, wherein said implant carrier comprises a holding member selected from a group consisting of an anchoring structure, a compliance mechanism and any combination thereof.

It is another object of the present invention to disclose the two parts insertion tool as defined above, wherein said anchoring structure contacts said implant at a location on said implant selected from a group consisting of a proximal portion of said implant, a central portion of said implant, a distal portion of said implant and any combination thereof.

It is another object of the present invention to disclose the two parts insertion tool as defined above, wherein said anchoring structure is configured to remove said implant from said implant carrier by a means selected from a group consisting of pulling said implant from said implant carrier and pushing implant from said implant carrier.

It is another object of the present invention to disclose the two parts insertion tool as defined above, wherein said anchoring structure is configured to enable release of said implant from said implant carrier by ending of contact between said holding member and said implant.

It is another object of the present invention to disclose the two parts insertion tool as defined above, wherein said anchoring structure comprises at least one tooth.

It is another object of the present invention to disclose the two parts insertion tool as defined above, wherein said anchoring structure retains said implant on said implant carrier before use.

It is another object of the present invention to disclose the two parts insertion tool as defined above, wherein said anchoring structure lifts said implant off of said implant carrier to allow removal of said implant from said implant carrier.

It is another object of the present invention to disclose the two parts insertion tool as defined above, additionally comprising a fluid inlet, said fluid inlet in fluid communication with said implant carrier.

It is another object of the present invention to disclose the two parts insertion tool as defined above, additionally comprising a cover tube configured to retain said carrier tube and said arm in said first configuration.

It is another object of the present invention to disclose the two parts insertion tool as defined above, wherein said cover tube is movable from said bendable region, removal of said cover tube from said bendable region enabling transfer of said carrier tube and said arm to said second configuration.

It is another object of the present invention to disclose the two parts insertion tool as defined above, wherein, in said first configuration, said implant carrier has a diameter less than that of said carrier tube.

It is another object of the present invention to disclose the two parts insertion tool as defined above, wherein, in said second configuration, said implant carrier has a curvature such that a distance from a bottom center of said implant carrier to a top edge of said implant carrier is a range from 0 to 1 cm.

It is another object of the present invention to disclose the two parts insertion tool as defined above, wherein said transfer of said implant carrier from said first position to said second position is reversible.

It is another object of the present invention to disclose the two parts insertion tool as defined above, wherein said transfer of said implant carrier from said first position to said second position is effectuated by a means selected from a group consisting of pulling at least a portion of said implant carrier and pushing at least a portion of said implant carrier.

It is another object of the present invention to disclose the two parts insertion tool as defined above, wherein said transfer of said implant carrier from said first configuration to said second configuration is by means of wires.

It is another object of the present invention to disclose the two parts insertion tool as defined above, wherein said wires comprise a shape memory material.

It is another object of the present invention to disclose the two parts insertion tool as defined above, wherein said wires comprise Nitinol.

It is another object of the present invention to disclose the two parts insertion tool as defined above, wherein, in said second configuration, a length of said implant carrier is in a range from 1.5 mm to 12 mm.

It is another object of the present invention to disclose the two parts insertion tool as defined above, wherein said implant carrier is configured to hold a plurality of implants.

It is another object of the present invention to disclose the two parts insertion tool as defined above, wherein said implant carrier for a plurality of implants is a case with shelves.

It is another object of the present invention to disclose the two parts insertion tool as defined above, wherein said implant is movable by means of wires.

It is another object of the present invention to disclose the two parts insertion tool as defined above, wherein said wires are attachable to said implant by means of a group consisting of ears and holes in said implant.

It is another object of the present invention to disclose the two parts insertion tool as defined above, wherein said insertion tool is configured to perform at least one function selected from a group consisting of emplace an ophthalmic implant, emplace an ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof.

It is another object of the present invention to disclose the two parts insertion tool as defined above, wherein said carrier arm wire has a width in a range from 50 μm to 0.5 mm.

It is another object of the present invention to disclose the two parts insertion tool as defined above, wherein said carrier arm wire has a length in a range from 5 cm to 6 cm.

It is another object of the present invention to disclose the two parts insertion tool as defined above, wherein the implant is either loaded into the insertion tool at a manufacturing site or loaded into the insertion tool at the operation site.

It is another object of the present invention to disclose the two parts insertion tool as defined above, wherein said insertion tool is configured to provide a slow and constant flow of fluid through the insertion tool.

It is another object of the present invention to disclose the two parts insertion tool as defined above, wherein said flow of fluid is in a range of 0.05 ml/min to 1 ml/min.

It is another object of the present invention to disclose the two parts insertion tool as defined above, wherein said implant is loadable into said insertion tool by transforming said insertion tool to said first body configuration.

It is another object of the present invention to disclose the two parts insertion tool as defined above, wherein said implant is loadable into said insertion tool by transforming said implant carrier to said first implant carrier configuration.

It is another object of the present invention to disclose the two parts insertion tool as defined above, wherein said insertion tool is connectable to an operating room facility, said operating room facility selected from a group consisting of a fluid source, a positioning tool, a power supply, a light source, and any combination thereof.

It is another object of the present invention to disclose the two parts insertion tool as defined above, wherein said implant is separated from a larger implant sheet by a means selected from a group consisting of punching, cutting, microdissection, laser ablation and any combination thereof.

It is another object of the present invention to disclose the two parts insertion tool as defined above, wherein said separation of said implant from said larger implant sheet is performed while the implant is resting on or located above said implant carrier.

It is another object of the present invention to disclose the two parts insertion tool as defined above, wherein a material of said implant carrier is configured to resist damage during laser ablation or microdissection processing to separate said implant from said larger implant sheet by having a property selected from a group consisting of reflecting laser light, being transparent to laser light, being resistive to high temperatures, having a protection layer and any combination thereof.

It is another object of the present invention to disclose the two parts insertion tool as defined above, wherein said material of said implant carrier is configured to resist damage during cutting or punching to separate said implant from said larger implant sheet by having a property selected from a group consisting of having a mechanical yield strain of at least 50%, having an elastic modulus of at least 5 GPa, having a Shore durometer hardness type D of at least 50 and any combination thereof.

It is another object of the present invention to disclose the two parts insertion tool as defined above, additionally comprising a mechanism adapted to feedback of the carrier location.

It is another object of the present invention to disclose the two parts insertion tool as defined above, additionally comprising a mechanism adapted to feedback whether said implant is on said implant carrier.

It is another object of the present invention to disclose a method of inserting an implant, comprising steps of:
providing an insertion tool for inserting an implant, said insertion tool is characterized by a distal part and a proximal part reversibly interconnected to each other; said distal part comprises (a) an implant carrier configured to hold an implant; (b) a body having at least two configurations, a first body configuration with said implant carrier at least partially contained within said body and said implant held by said implant carrier and a second body configuration with said implant carrier outside said body;
said proximal part comprises a proximal part body, adapted to be sealingly reversibly connected to said body in said distal part;
wherein, when implantation is required, sealingly coupling said distal part to said proximal part.

It is another object of the present invention to disclose the method as defined above, additionally comprising step of cryopreservation said distal part.

It is another object of the present invention to disclose the method as defined above, additionally comprising step of, when implantation is required, thawing said distal part.

It is another object of the present invention to disclose the method as defined above, wherein said implant having an implant lower side and an implant upper side, said implant upper side comprising cells.

It is another object of the present invention to disclose the method as defined above, additionally comprising step of emplacing said implant on said implant carrier.

It is another object of the present invention to disclose the method as defined above, additionally comprising step of cryopreserving said distal part.

It is another object of the present invention to disclose the method as defined above, additionally comprising step of extracting said distal part from said cryopreserving prior to implantation of said implant.

It is another object of the present invention to disclose the method as defined above, additionally comprising steps of
making an incision in the eyeball;
inserting the tip of said insertion tool passing through said incision;
transferring said body from said first body configuration to said second body configuration;
transferring said implant carrier from a first carrier configuration to a second carrier configuration;
removing said implant from said implant carrier into said bleb;
wherein said implant carrier is configured to maintain a position of the implant and a shape of the implant and functionality of said cells during such time as the implant carrier is contained by said body, and during an implantation procedure;
wherein, in said first carrier configuration, the implant carrier has a shape selected from a group consisting of folded, rolled and bent, and, in said second carrier configuration, the implant carrier has a curved shape;
further wherein said implant carrier is in said first carrier configuration at such times as said implant carrier is within said body and said implant carrier is transformable to said second carrier configuration during such times as said implant carrier is outside of said body.

It is another object of the present invention to disclose the method as defined above, additionally comprising at least one step selected from (a) inserting an incision tool, said incision tool making an incision in a retina;
(b) removing said incision tool;
(c) inserting a second tool and creating a bleb in said retina, said bleb within said retina and in contact with said incision in said retina;
(d) removing said second tool;
(e) removing said insertion tool;
(f) flattening said bleb;
(g) sealing said incision in said retina; and,
(h) sealing said incision in said eyeball;
(i) any combination thereof.

It is another object of the present invention to disclose the method as defined above, additionally comprising a step of retracting said implant carrier into said body.

It is another object of the present invention to disclose the method as defined above, additionally comprising a step of moving said implant carrier outward from said body.

It is another object of the present invention to disclose the method as defined above, additionally comprising a step of opening said body.

It is another object of the present invention to disclose the method as defined above, additionally comprising a step of providing said implant carrier as a part of said body.

It is another object of the present invention to disclose the method as defined above, additionally comprising a step of providing said implant carrier comprising a member of a group consisting of Nitinol, Polyimide (Kapton), stainless steel and any combination thereof.

It is another object of the present invention to disclose the method as defined above, additionally comprising a step of providing said implant carrier comprising a coating selected from a group consisting of a hydrophilic coating, a hydrophobic coating and any combination thereof.

It is another object of the present invention to disclose the method as defined above, additionally comprising a step of making said implant of a material selected from a group consisting of soft, foldable, bendable and any combination thereof.

It is another object of the present invention to disclose the method as defined above, additionally comprising steps of providing an implant removal mechanism and of selecting said implant removal mechanism from a group consisting of an arm contactable to said implant, a wire attachable to said implant, fluid flow, vibration, and any combination thereof.

It is another object of the present invention to disclose the method as defined above, wherein said fluid flow is adapted to perform at least one selected from a group consisting of lifting said implant from said implant carrier, pushing said implant out of said tool, remove all air bubbles in said tool, and any combination thereof.

It is another object of the present invention to disclose the method as defined above, additionally comprising at least one element, adapted to direct said fluid flow to a predetermined direction.

It is another object of the present invention to disclose the method as defined above, additionally comprising at least one element, adapted to elevate said implant from said graft carrier by allowing said fluid to flow below said implant; thereby facilitating extraction therefrom.

It is another object of the present invention to disclose the method as defined above, wherein said vibration is provided by at least one selected from a group consisting of tapping, electric motor, piezoelectric transducer, ultrasound means and any combination thereof.

It is another object of the present invention to disclose the method as defined above, additionally comprising means adapted to increase said fluid flow velocity.

It is another object of the present invention to disclose the method as defined above, wherein said increase in said fluid flow velocity is provided by means selected from a group consisting of narrowing said body, enlarging said body, blocking part thereof, adding an elevated or protruding part inside or at the opening of said body, or any combination thereof.

It is another object of the present invention to disclose the method as defined above, wherein said fluid flow is controlled either manually or automatically.

It is another object of the present invention to disclose the method as defined above, additionally comprising a step of configuring said implant carrier to position said implant into a member of a group consisting of a subretinal bleb and an anterior chamber.

It is another object of the present invention to disclose the method as defined above, additionally comprising a step of configuring said implant for ophthalmic applications.

It is another object of the present invention to disclose the method as defined above, additionally comprising a step of selecting said implant to be a retinal implant.

It is another object of the present invention to disclose the method as defined above, additionally comprising a step of selecting said implant to be a corneal implant.

It is another object of the present invention to disclose the method as defined above, additionally comprising a step of selecting said implant comprising a member of a group consisting of retinal pigment epithelium cells, photo-receptor cells and any combination thereof.

It is another object of the present invention to disclose the method as defined above, additionally comprising a step of selecting said implant comprising a member of a group consisting of collagen, hydrogel, a medication and any combination thereof.

It is another object of the present invention to disclose the method as defined above, wherein said medication is a slow-release medication.

It is another object of the present invention to disclose the method as defined above, additionally comprising a step of said implant diameter is in a range of 0.5 mm to 8 mm.

It is another object of the present invention to disclose the method as defined above, additionally comprising a step of said implant thickness is in a range of 2 μm to 500 μm.

It is another object of the present invention to disclose the method as defined above, additionally comprising a step of said implant length is in a range of 0.5 mm to 8 mm.

It is another object of the present invention to disclose the method as defined above, additionally comprising a step of said implant width is in a range of 0.5 mm to 8 mm.

It is another object of the present invention to disclose the method as defined above, additionally comprising a step of inserting a trocar through said incision in said eye.

It is another object of the present invention to disclose the method as defined above, additionally comprising a step of selecting the trocar diameter to be no larger than 20 G (0.9 mm).

It is another object of the present invention to disclose the method as defined above, additionally comprising a step of selecting the trocar diameter to be larger than 20 G (0.9 mm).

It is another object of the present invention to disclose the method as defined above, additionally comprising a step of effectuating actuation of removal of said implant from said implant carrier is by a mechanism comprising a member of a group consisting of a slider, a pin, a converter from circular motion to linear motion, a motor and gear, a motor and cam, a wheel, a knob, a screw, and any combination thereof.

It is another object of the present invention to disclose the method as defined above, additionally comprising a step of providing a protective seal at a distal end of said insertion tool.

It is another object of the present invention to disclose the method as defined above, additionally comprising a step of selecting said protective seal from a group consisting of a cap, a diaphragm and any combination thereof.

It is another object of the present invention to disclose the method as defined above, additionally comprising a step of said protective seal allowing at least one of a group consisting of diffusion of gas therethrough and a nutrient path therethrough.

It is another object of the present invention to disclose the method as defined above, additionally comprising a step of fluid flow performing a member of a group consisting of detaching said implant from said implant carrier, positioning said implant, transferring liquid, maintaining viability of said implant, maintaining intraocular pressure, maintaining retinal bleb size, and any combination thereof.

It is another object of the present invention to disclose the method as defined above, additionally comprising steps of emplacing said implant within said insertion tool prior to shipment and of maintaining said implant in a usable condition between said emplacing and use.

It is another object of the present invention to disclose the method as defined above, additionally comprising a step of maintaining said implant in a temperature range of 4° C. to 25° C. between said emplacing and said use.

It is another object of the present invention to disclose the method as defined above, additionally comprising a step of maintaining said implant in a temperature range of 30° C. to 38° C. between said emplacing within said insertion tool and said use.

It is another object of the present invention to disclose the method as defined above, additionally comprising a step of maintaining said implant in solution between said emplacing within said insertion tool and said use.

It is another object of the present invention to disclose the method as defined above, additionally comprising a step of selecting said fluid comprising a member of a group consisting of balanced saline solution (BSS), BSS Plus, BSS Plus with additional viscous content such as Hyaluronic acid, and a CO₂ independent medium.

It is another object of the present invention to disclose the method as defined above, additionally comprising a step of selecting a shape of a portion of said body from a group consisting of bent, controllably bendable, straight and any combination thereof.

It is another object of the present invention to disclose the method as defined above, additionally comprising a step of passing said bent or controllably bendable portion through at least one of a trocar and a cannula.

It is another object of the present invention to disclose the method as defined above, additionally comprising a step of selecting, in said bent configuration, an angle between a longitudinal axis of said body and a longitudinal axis of said bent portion to be in a range from 0° to 90°.

It is another object of the present invention to disclose the method as defined above, additionally comprising a step of selecting at least a portion of a member of a group consisting of said body, said cap, said implant carrier and any combination thereof to be transparent.

It is another object of the present invention to disclose the method as defined above, additionally comprising a step of said transparency enabling visibility of at least a portion of a member of a group consisting of said implant, said implant carrier and any combination thereof.

It is another object of the present invention to disclose the method as defined above, additionally comprising a step of coloring at least a portion of said insertion tool to improve visibility of a member of a group consisting of said implant, at least a portion of a component of said insertion tool and any combination thereof.

It is another object of the present invention to disclose the method as defined above, additionally comprising a step of selecting said color from a member of a group consisting of darker than another component, lighter than another component, fluorescent, green, and any combination thereof.

It is another object of the present invention to disclose the method as defined above, additionally comprising a step of selecting control of said insertion tool from a group consisting of robotic, automatic, semi-automatic, manual, vibration damping and any combination thereof.

It is another object of the present invention to disclose the method as defined above, additionally comprising a step of providing a lighting mechanism configured to provide light at a distal end of said insertion tool.

It is another object of the present invention to disclose the method as defined above, additionally comprising a step of selecting said lighting mechanism comprising a member of a group consisting of an LED, a fiber optic cable, and any combination thereof.

It is another object of the present invention to disclose the method as defined above, additionally comprising a step of providing a camera configured to display a view of a region near a distal portion of said insertion tool.

It is another object of the present invention to disclose the method as defined above, additionally comprising a step of providing a mechanism for rotating said implant while maintaining a direction of implantation.

It is another object of the present invention to disclose the method as defined above, additionally comprising a step of ejecting said implant from said implant carrier.

It is another object of the present invention to disclose the method as defined above, additionally comprising a step of providing said implant carrier in contact with only said implant lower side.

It is another object of the present invention to disclose the method as defined above, additionally comprising a step of configuring said implant carrier to protect said implant upper side from mechanical stress and shear.

It is another object of the present invention to disclose the method as defined above, additionally comprising steps of releasably connecting the implant to the implant carrier, and configuring said releasable connection, before release, to prevent a member of a group consisting of movement of said implant, folding of said implant and any combination thereof.

It is another object of the present invention to disclose the method as defined above, additionally comprising a step of providing a locking mechanism.

It is another object of the present invention to disclose the method as defined above, additionally comprising a step of providing said locking mechanism comprising a member of a group consisting of locking tubes comprising holes through which a fluid can be passed, an arm contactable to said implant, a wire attachable to said implant, and any combination thereof,

It is another object of the present invention to disclose the method as defined above, additionally comprising a step of flowing fluid into said insertion tool from a volume near a distal end of said insertion tool.

It is another object of the present invention to disclose the method as defined above, additionally comprising a step of configuring at least a portion of said insertion tool to enable implant orientation by means of rotation of said at least a portion of said insertion tool.

It is another object of the present invention to disclose the method as defined above, additionally comprising a step of providing an insertion stopper, configured to allow adjustment of a distance said implant carrier extends distally from said body of said insertion tool.

It is another object of the present invention to disclose the method as defined above, additionally comprising a step of extending said implant carrier from said body by at least one predetermined distance.

It is another object of the present invention to disclose the method as defined above, additionally comprising a step of said at least one predetermined distance comprising a first predetermined distance and a second predetermined distance.

It is another object of the present invention to disclose the method as defined above, additionally comprising a step of selecting said first predetermined distance to be in a range from 7 mm to 10 mm and said second predetermined distance to be in a range from 2 to 6 mm.

It is another object of the present invention to disclose the method as defined above, additionally comprising a step of selecting said extending mechanism from a group consisting of a slider, a pin, a converter from circular motion to linear motion, a motor and gear, a motor and cam, and any combination thereof.

It is another object of the present invention to disclose the method as defined above, additionally comprising a step of providing said carrier tube comprising a superelastic material.

It is another object of the present invention to disclose the method as defined above, additionally comprising a step of providing said carrier tube comprising Nitinol.

It is another object of the present invention to disclose the method as defined above, additionally comprising a step of providing said implant carrier comprising a superelastic material.

It is another object of the present invention to disclose the method as defined above, additionally comprising a step of providing said implant carrier comprising Nitinol.

It is another object of the present invention to disclose the method as defined above, additionally comprising a step of providing said implant carrier comprising holes.

It is another object of the present invention to disclose the method as defined above, additionally comprising a step of providing said implant carrier comprising a member of a group consisting of an anchoring structure, a compliance mechanism and any combination thereof.

It is another object of the present invention to disclose the method as defined above, additionally comprising a step of said anchoring structure contacting a location on said implant selected from a group consisting of a proximal portion of said implant, a central portion of said implant, a distal portion of said implant and any combination thereof.

It is another object of the present invention to disclose the method as defined above, additionally comprising a step of configuring said anchoring structure to remove said implant from said implant carrier by a means selected from a group consisting of pulling said implant from said implant carrier and pushing implant from said implant carrier.

It is another object of the present invention to disclose the method as defined above, additionally comprising a step of releasing said implant from said implant carrier by ending contact between said holding member and said implant.

It is another object of the present invention to disclose the method as defined above, additionally comprising a step of said anchoring structure comprising at least one tooth.

It is another object of the present invention to disclose the method as defined above, additionally comprising a step of said anchoring structure retaining said implant on said implant carrier before use.

It is another object of the present invention to disclose the method as defined above, additionally comprising a step of said anchoring structure lifting said implant off of said implant carrier, thereby allowing removal of said implant from said implant carrier.

It is another object of the present invention to disclose the method as defined above, additionally comprising a step of providing a fluid inlet, said fluid inlet in fluid communication with said implant carrier.

It is another object of the present invention to disclose the method as defined above, additionally comprising a step of providing a cover tube configured to retain said carrier tube and said arm in said first configuration.

It is another object of the present invention to disclose the method as defined above, additionally comprising a step of said cover tube is movable from said bendable region, removal of said cover tube from said bendable region enabling transfer of said carrier tube and said arm to said second configuration.

It is another object of the present invention to disclose the method as defined above, additionally comprising a step of selecting, in said first configuration, said implant carrier to have a diameter less than that of said carrier tube.

It is another object of the present invention to disclose the method as defined above, additionally comprising a step of selecting, in said second configuration, a curvature said implant carrier to be such that a distance from a bottom center of said implant carrier to a top edge of said implant carrier is a range from 0 to 1 cm.

It is another object of the present invention to disclose the method as defined above, additionally comprising a step of configuring said transfer of said implant carrier from said first position to said second position to be reversible.

It is another object of the present invention to disclose the method as defined above, additionally comprising a step of effectuating said transfer of said implant carrier from said first position to said second position by a means selected from a group consisting of pulling at least a portion of said implant carrier and pushing at least a portion of said implant carrier.

It is another object of the present invention to disclose the method as defined above, additionally comprising a step of transferring said implant carrier from said first configuration to said second configuration by means of wires.

It is another object of the present invention to disclose the method as defined above, additionally comprising a step of comprising said wires of shape memory alloy.

It is another object of the present invention to disclose the method as defined above, additionally comprising a step of comprising said wires of Nitinol.

It is another object of the present invention to disclose the method as defined above, additionally comprising a step of selecting, in said second configuration, a length of said implant carrier to be in a range from 1.5 mm to 12 mm.

It is another object of the present invention to disclose the method as defined above, additionally comprising a step of configuring said implant carrier to hold a plurality of implants.

It is another object of the present invention to disclose the method as defined above, additionally comprising a step of selecting said implant carrier for a plurality of implants to be a case with shelves.

It is another object of the present invention to disclose the method as defined above, additionally comprising a step of moving said implant by means of wires.

It is another object of the present invention to disclose the method as defined above, additionally comprising a step of attaching said wires to said implant by means of a group consisting of ears and holes in said implant.

It is another object of the present invention to disclose the method as defined above, additionally comprising a step of configuring said insertion tool to perform at least one function selected from a group consisting of emplace an ophthalmic implant, emplace an ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof.

It is another object of the present invention to disclose the method as defined above, additionally comprising a step of selecting said carrier arm wire to have a width in a range from 50 μm to 0.5 mm.

It is another object of the present invention to disclose the method as defined above, additionally comprising a step of selecting said carrier arm wire to have a length in a range from 5 cm to 6 cm.

It is another object of the present invention to disclose the method as defined above, additionally comprising a step of loading the implant into the insertion tool either at a manufacturing site or at the operation site.

It is another object of the present invention to disclose the method as defined above, additionally comprising a step of providing a slow and constant flow of fluid through the insertion tool.

It is another object of the present invention to disclose the method as defined above, additionally comprising a step of providing said flow of fluid in a range of 0.05 ml/min to 1 ml/min.

It is another object of the present invention to disclose the method as defined above, additionally comprising a step of flowing said of fluid via a cannula.

It is another object of the present invention to disclose the method as defined above, additionally comprising a step of cutting said implant from a larger implant sheet by a means selected from a group consisting of punching, cutting, microdissection, laser ablation and any combination thereof.

It is another object of the present invention to disclose the method as defined above, additionally comprising a step of performing said cutting of said implant from said larger implant sheet while the implant is resting on or located above said implant carrier.

It is another object of the present invention to disclose the method as defined above, additionally comprising a step of selecting a material of said implant carrier configured to resist damage during laser ablation or microdissection processing to separate said implant from said larger implant sheet having a property selected from a group consisting of reflecting laser light, being transparent to laser light, being resistive to high temperatures, having a protection layer and any combination thereof.

It is another object of the present invention to disclose the method as defined above, additionally comprising a step of selecting said material of said implant carrier configured to resist damage during cutting or punching of said implant from said larger implant sheet having a property selected from a group consisting of having a mechanical yield strain of at least 50%, having an elastic modulus of at least 5 GPa, having a Shore durometer hardness type D of at least 50 and any combination thereof.

It is another object of the present invention to disclose the method as defined above, additionally comprising a step of loading said implant into said insertion tool by transforming said insertion tool to said first body configuration.

It is another object of the present invention to disclose the method as defined above, additionally comprising a step of loading said implant into said insertion tool by transforming said implant carrier to said first implant carrier configuration.

It is another object of the present invention to disclose the method as defined above, additionally comprising steps of connecting said insertion tool to an operating room facility, and selecting said operating room facility from a group consisting of a fluid source, a positioning tool, a power supply, a light source, and any combination thereof.

It is another object of the present invention to disclose the method as defined above, additionally comprising a mechanism adapted to feedback of the carrier location.

It is another object of the present invention to disclose the method as defined above, additionally comprising a mechanism adapted to feedback whether said implant is on said implant carrier.

It should be noted that according to one embodiment of the present invention, the graft/implant 100 is printed directly on the carrier 1290 either just before the implantation or in advance (and cryopreserved until implantation).

Although certain embodiments have been illustrated and described herein, it will be appreciated by those of ordinary skill in the art that a wide variety of alternative or equivalent embodiments or implementations, calculated to achieve the same or similar purposes, may be substituted for the embodiments illustrated and described herein without departing from the scope of the present invention. Those of skill in the art will readily appreciate that embodiments in accordance with the present invention may be implemented in a very wide variety of ways. This application is intended to cover any and all adaptations and/or variations of the embodiments discussed herein.

The terms and expressions which have been employed in the foregoing specification are used therein as terms of description and not of limitation, and there is no intention, in the use of such terms and expressions, to exclude equivalents of the features shown and/or described or portions thereof, it being recognized that the scope of the invention is defined and limited only by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

1.-215. (canceled)

216. An insertion tool for inserting an implant, said implant having an implant lower side and an implant upper side, said implant upper side comprising cells, said insertion tool comprising:
an implant carrier configured to hold said implant; and
a body having at least two configurations, a first body configuration with said implant carrier at least partially contained within said body and said implant held by said implant carrier and a second body configuration with said implant carrier outside said body;
said implant carrier is configured to maintain a position of the implant and a shape of the implant and functionality of said cells during such time as the implant carrier is contained by said body, and during an implantation procedure;
wherein said implant carrier has at least two configurations, a first carrier configuration where the implant carrier has a shape selected from a group consisting of folded, rolled and bent, and a second carrier configuration where the implant carrier has a substantially non flat shape;
further wherein said implant carrier is in said first carrier configuration at such times as said implant carrier is within said body and said implant carrier is transformable to said second carrier configuration during such times as said implant carrier is outside of said body;
further wherein said implant carrier and said implant, during implantation, is transferred to said second configuration.

217. The tool of claim 216, wherein at least one of the following is being held true (a) said implant carrier is retractable into said body; (b) said implant carrier is movable outward from said body; (c) said body is openable; (d) said implant carrier is a part of said body; (e) said implant carrier comprises a coating selected from a group consisting of a hydrophilic coating, a hydrophobic coating and any combination thereof; and any combination thereof.

218. The tool of claim 216, additionally comprising an implant removal mechanism selected from a group consisting of an arm contactable to said implant, a wire attachable to said implant, fluid flow, vibration, and any combination thereof.

219. The tool of claim 218, wherein at least one of the following is held true (a) said fluid flow is adapted to perform at least one selected from a group consisting of lifting said implant from said implant carrier, pushing said implant out of said tool, remove all air bubbles in said tool, and any combination thereof; (b) said tool additionally comprising at least one element, adapted to direct said fluid flow to a predetermined direction; (c) said tool additionally comprising at least one element, adapted to elevate said implant from said graft carrier, allowing said fluid to flow below said implant; (d) said vibration is provided by at least one selected from a group consisting of tapping, electric motor, piezoelectric transducer, ultrasound means and any combination thereof; (e) said tool additionally comprising means adapted to increase said fluid flow velocity; said increase in said fluid flow velocity is provided by means selected from a group consisting of narrowing said body, enlarging said body, blocking part thereof, adding an elevated or protruding part inside or at the opening of said body, or any combination thereof; any combination thereof.

220. The tool of claim 216, wherein said implant is configured for ophthalmic applications; further wherein at least one of the following is held true (a) said implant is selected from a group consisting of retinal implant, corneal implant and any combination thereof; (b) said implant comprises a member of a group consisting of retinal pigment epithelium cells, photo-receptor cells and any combination thereof; and any combination thereof.

221. The tool of claim 216, wherein said implant comprises a member of a group consisting of collagen, hydrogel, a medication, a slow-release medication and any combination thereof.

222. The tool of claim 216, wherein actuation of removal of said implant from said implant carrier is effectuated by a mechanism comprising a member of a group consisting of a slider, a pin, a converter from circular motion to linear motion, a motor and gear, a motor and cam, a wheel, a knob, a screw, and any combination thereof.

223. The tool of claim 216, additionally comprising a protective seal at a distal end of said insertion tool; further wherein at least one of the following is held true (a) selected from a group consisting of a cap, a diaphragm and any combination thereof; (b) said protective seal allows at least one of a group consisting of diffusion of gas therethrough and a nutrient path therethrough; and any combination thereof.

224. The tool of claim 216, wherein a shape of a portion of said body is selected from a group consisting of bent, controllably bendable, straight and any combination thereof; further wherein said bent or controllably bendable portion is passable through at least one of a trocar and a cannula; further wherein, in said bent configuration, an angle between a longitudinal axis of said body and a longitudinal axis of said bent portion is in a range from 0° to 90°.

225. The tool of claim 216, wherein at least a portion of a member of a group consisting of said body, said cap, said implant carrier and any combination thereof is transparent; wherein said transparency enables visibility of at least a portion of a member of a group consisting of said implant, said implant carrier and any combination thereof.

226. The tool of claim 216, wherein at least one of the following is held true (a) control of said insertion tool is selected from a group consisting of robotic, automatic, semi-automatic, manual, vibration damping and any combination thereof; (b) said tool additionally comprising a camera configured to display a view of a region near a distal portion of said insertion tool; (c) said tool additionally comprising a mechanism for rotating said implant while maintaining a direction of implantation; (d) wherein the implant is releasably connectable to the implant carrier, said releasable connection configured, before release, to prevent a member of a group consisting of movement of said implant, folding of said implant and any combination thereof (e) said tool additionally comprising a locking mechanism; said locking mechanism comprises a member of a group consisting of locking tubes comprising holes through which a fluid can be passed, an arm contactable to said implant, a wire attachable to said implant, and any combination thereof; and any combination thereof.

227. The tool of claim 216, additionally comprising a lighting mechanism configured to provide light at a distal end of said insertion tool; further wherein said lighting mechanism comprises a member of a group consisting of an LED, a fiber optic cable, and any combination thereof.

228. The tool of claim 216, at least one of the following is held true (a) said tool additionally comprising an insertion stopper, configured to allow adjustment of a distance said implant carrier extends distally from said body of said insertion tool; (b) said implant carrier is extendable from said body by at least one predetermined distance; (c) said tool additionally comprising a cover tube configured to retain said carrier tube and said arm in said first configuration; further wherein said cover tube is movable from said bendable region, removal of said cover tube from said bendable region enabling transfer of said carrier tube and said arm to said second configuration; further wherein, in said first configuration, said implant carrier has a diameter less than that of said carrier tube; and any combination thereof.

229. The tool of claim 216, wherein said implant carrier comprises a holding member selected from a group consisting of an anchoring structure, a compliance mechanism and any combination thereof; wherein at least one of the following is held true (a) said anchoring structure contacts said implant at a location on said implant selected from a group consisting of a proximal portion of said implant, a central portion of said implant, a distal portion of said implant and any combination thereof; (b) said anchoring structure is configured to remove said implant from said implant carrier by a means selected from a group consisting of pulling said implant from said implant carrier and pushing implant from said implant carrier; (c) said anchoring structure is configured to enable release of said implant from said implant carrier by ending of contact between said holding member and said implant; (d) said anchoring structure comprises at least one tooth; (e) said anchoring structure retains said implant on said implant carrier before use; (f) said anchoring structure lifts said implant off of said implant carrier to allow removal of said implant from said implant carrier; and any combination thereof.

230. The tool of claim 216, wherein said transfer of said implant carrier from said first configuration to said second configuration is reversible; further wherein said transfer of said implant carrier from said first configuration to said second configuration is effectuated by a means selected from a group consisting of pulling at least a portion of said implant carrier and pushing at least a portion of said implant carrier.

231. The tool of claim 216, wherein at least one of the following is held true (a) said insertion tool is configured to perform at least one function selected from a group consisting of emplace an ophthalmic implant, emplace an ophthalmic device, inject an ophthalmic drug, emplace a drug delivery device, emplace a nerve patch, emplace a cardiac patch, emplace a pancreatic patch, emplace pancreatic tissue, emplace a liver patch, emplace liver tissue, and any combination thereof; (b) wherein said insertion tool is configured to provide a slow and constant flow of fluid through the insertion tool; said flow of fluid is in a range of 0.05 ml/min to 1 ml/min; and any combination thereof.

232. The tool of claim 216, additionally comprising a mechanism adapted to feedback at least one selected from a group consisting of (a) the carrier location; (b) whether said implant is on said implant carrier; and any combination thereof.

233. A method of inserting an implant, comprising steps of:
providing an insertion tool for inserting an implant, said implant having an implant lower side and an implant upper side, said implant upper side comprising cells, said insertion tool comprising:
an implant carrier configured to hold said implant; and
a body having at least two configurations, a first body configuration with said implant carrier at least partially contained within said body and said implant held by said implant carrier and a second body configuration with said implant carrier outside said body;
transferring said implant carrier from a first carrier configuration to a second carrier configuration, while said implant is on said implant carrier;
removing said implant from said implant carrier into the bleb;
wherein said implant carrier is configured to maintain a position of the implant and a shape of the implant and functionality of said cells during such time as the implant carrier is contained by said body, and during an implantation procedure;
wherein, in said first carrier configuration, the implant carrier has a shape selected from a group consisting of folded, rolled and bent, and, in said second carrier configuration, the implant carrier has a curved shape;
further wherein said implant carrier is in said first carrier configuration at such times as said implant carrier is within said body and said implant carrier is transformable to said second carrier configuration during such times as said implant carrier is outside of said body.

234. The method of claim 233, additionally comprising at least one step selected from
(a) inserting an incision tool, said incision tool making an incision in a retina;
(b) emplacing said implant on said implant carrier;
(c) making an incision in the eyeball;
(d) inserting the tip of said insertion tool passing through said incision;
(e) removing said incision tool;
(f) inserting a second tool and creating a bleb in said retina, said bleb within said retina and in contact with said incision in said retina;
(g) removing said second tool;
(h) removing said insertion tool;
(i) flattening said bleb;
(j) sealing said incision in said retina; and,
(k) sealing said incision in said eyeball;
(l) any combination thereof.
